(19) United States
(12) Patent Application Publication
(10) Pub. No.: US 2016/0368872 A1
(43) Pub. Date: Dec. 22, 2016

(54) COMPOUNDS AND METHODS FOR TREATING CANCERS

(71) Applicants: HEALTH RESEARCH, INC., Buffalo, NY (US); PANACELA LABS LLC, Moscow (RU)

(72) Inventors: Katerina GUROVA, Orchard Park, NY (US); Warren WADE, San Diego, CA (US)

(21) Appl. No.: 14/775,748

(22) PCT Filed: Mar. 14, 2014
(86) PCT No.: PCT/US14/28863

§ 371 Date: Sep. 14, 2015

Related U.S. Application Data
(60) Provisional application No. 61/781,423, filed on Mar. 14, 2013.

(51) Int. Cl. C07D 209/88; C07D 491/048; C07D 471/04; C07D 498/14; C07D 491/147; C07D 471/14; C07D 498/04; C07D 487/04; C07D 491/052

(52) U.S. Cl. CPC C07D 209/88; C07D 487/04; C07D 491/048; C07D 471/04; C07D 491/052; C07D 491/147; C07D 471/14; C07D 498/04; C07D 498/14

(57) ABSTRACT

Provided are carbazole and carbazole-like compounds (e.g., pyridoindole and pyrrolodipyridine compounds. The compounds can be used to selectively kill cancer cells. Prostate cancer (PCa) is the most frequent neoplastic disease and the second leading cause of cancer-related deaths in men, claiming more than 30,000 men each year in the United States alone. PCa tumors are composed primarily of prostate luminal epithelial cells. Differentiation of prostate luminal epithelial cells is controlled in part by Androgen receptor (AR) driven expression of prostate-specific markers.

### CROSS-REFERENCE TO RELATED APPLICATIONS

This application claims priority to U.S. provisional patent application No. 61/781,423, filed Mar. 14, 2013, the disclosure of which is incorporated herein by reference.

### FIELD OF THE DISCLOSURE

The present disclosure generally relates to carbazole and carbazole-like compounds and methods of making and using such compounds.

### BACKGROUND OF THE DISCLOSURE

Prostate cancer (PCa) is the most frequent neoplastic disease and the second leading cause of cancer-related deaths in men, claiming more than 30,000 men each year in the United States alone. PCa tumors are composed primarily of prostate luminal epithelial cells. Differentiation of prostate luminal epithelial cells is controlled in part by Androgen receptor (AR) driven expression of prostate-specific markers. AR controls survival of the cells through mechanisms that remain unclear. In addition to prostate cancer, AR is in involved in the etiology of other cancers, including breast cancers. AR belongs to the family of steroid receptors and functions as a transcription factor. In the absence of ligand, members of this family are unstable proteins that reside in the cytoplasm bound to Heat Shock Protein 90 (Hsp90). Upon binding of a steroid such as androgen to the ligand binding domain (LBD) of AR, AR is freed from Hsp90 and translocates to the nucleus. Androgen-bound AR in the nucleus activates transcription of genes with androgen responsive elements (ARE) in their promoters (Cato, A. C., et al. 1998. Trends Endocrinol Metab 9: 150-154). In addition to its function as a transcriptional activator, AR is also capable of repressing transcription of some genes (Claessens, et al. 2001. J Steroid Biochem Mol Biol 76:23-30).

Depletion of androgens causes death of normal prostate luminal epithelial cells, which demonstrates the critical role of the AR pathway in their survival. Cancerous prostate cells continue to express AR and their survival also depends on the presence of androgens, which makes androgen deprivation the therapy of choice for patients with advanced PCa. Anti-androgen therapies, including use of the inhibitors flutamide and casodex, are usually effective initially, but rarely result in a complete cure. PCa relapse occurs in most of patients treated with such therapies, which leads to androgen-independent, chemotherapy-resistant tumors with poor prognosis. Thus, resistance to anti-androgen therapy is a major obstacle in successful treatment of PCa.

Analysis of the mechanisms of androgen-independence acquired by PCa during tumor progression indicates that loss of AR signalling is involved rarely (Balk, S. P. 2002. Urology 60: 132-138; discussion 138-139). On the contrary, androgen-independent PCa is typically characterized by heightened AR activity due to expression of AR mutants that are ligand-independent (constitutively active) or responsive to non-androgen ligands (Chen, Y., et al. 2008. Curr Opin Pharmacol 8:440-448; Tilley, et al. 1996. Clin Cancer Res 2:277-285; Koivisto, et al. 1998 Am J Pathol 152: 1-9; Marques, et al. 2005. Int J Cancer 117:221-229; Bohl, Cet al. 2005. J Biol Chem 280:37747-37754; Hara, T., et al. 2003. Cancer Res 63: 149-153).

It was recently shown that unlike normal prostate stem cells, prostate “tumor initiating cells” or “cancer stem cells”, a minor cell population believed to be the major source of self-renewing tumor cells, express functional AR (Vander Griend, et al. 2008. Cancer Res 68:9703-97111). This, together with the observed maintenance of AR activity in PCa tumors that have progressed to the stage of castration resistance, indicates that AR is a promising potential therapeutic target for both androgen-dependent and -independent PCa, as well as other AR positive cancer types. For example, breast epithelial cells are, in many regards, similar to prostate cells. As the survival of PC cells depend upon the androgen-stimulated activity AR, breast epithelial cells are similarly dependent upon the related estrogen (ER) and progesterone receptors (PR). The role of ER and PR in breast cancer (BC) and modulation of their function as a therapeutic approach has been the focus of studies for many years.

However, AR is expressed at low levels in normal mammary cells and at different levels in a majority of BCs, including 50% of “triple negative” (ER-, PR-, Her2-) BCs, for which targeted therapy is not yet available. Although the effect of androgens on breast epithelial cells has been addressed in several studies, the role played by AR in BC remains unclear (Birrell, et al. (1995) J Steroid Biochem Mol Biol 52, 459-467; Brettes, et al. (2008) Bull Cancer 95, 495-502; Di Monaco, et al. (1995) Anticancer Res 15, 2581-2584). Thus, current treatment modalities are largely ineffective for AR positive cancers, and there is an ongoing need for new methods for therapy of AR positive cancer cells, including but not limited to PCa and breast cancer.

### BRIEF SUMMARY OF THE DISCLOSURE

In an aspect, the present disclosure provides heterocyclic compounds having the following structure:

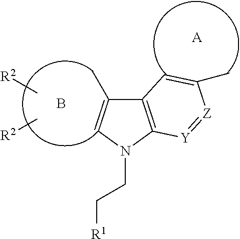

where R¹ is selected from the group consisting of a hydrogen atom, CH₃, CH₂F, CHF₂ and CF₃; R² is independently at each occurrence a hydrogen atom, halogen atom, —CN, —OH, substituted or unsubstituted alkyl, substituted or unsubstituted alkoxy group, —C(═O)N(R³)₂, —N(R³)₂, ketone, substituted or unsubstituted cycloalkyl group, or substituted or unsubstituted heterocycloalkyl group; Y and Z are independently a carbon or nitrogen atom; ring A is a substituted or unsubstituted 5 to 7 membered carbocyclic or heterocyclic ring; ring B is a substituted or unsubstituted 5 to 6 membered aryl or heteroaryl ring; and R³ is a hydrogen atom or substituted or unsubstituted alkyl. The compound has 0-2 R² groups.

In an embodiment, ring A is a 5 to 7 membered ring, for example a cyclic ketone, lactam, lactone, furanone, oxazolone, dioxolane, pyridinone, pyrimidinone, pyridazinone, dihydropyridazine, pyranone, or oxazinone. The 5 to 7 membered ring can be substituted with alkyl group(s) on carbon and/or nitrogen. In an embodiment, the compound has the following structure:

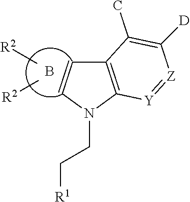

where C and D are replaced by the atoms of the following structures to form a ring:

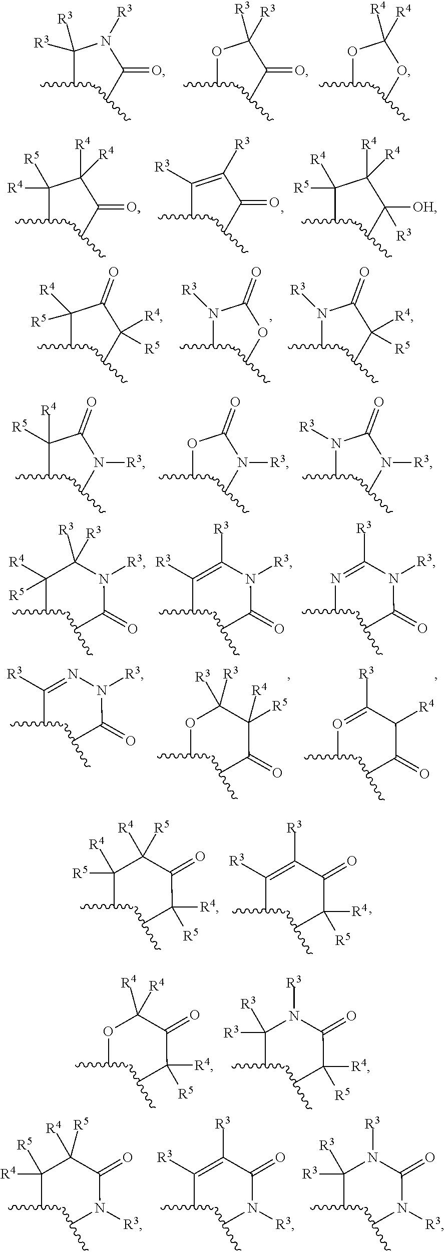

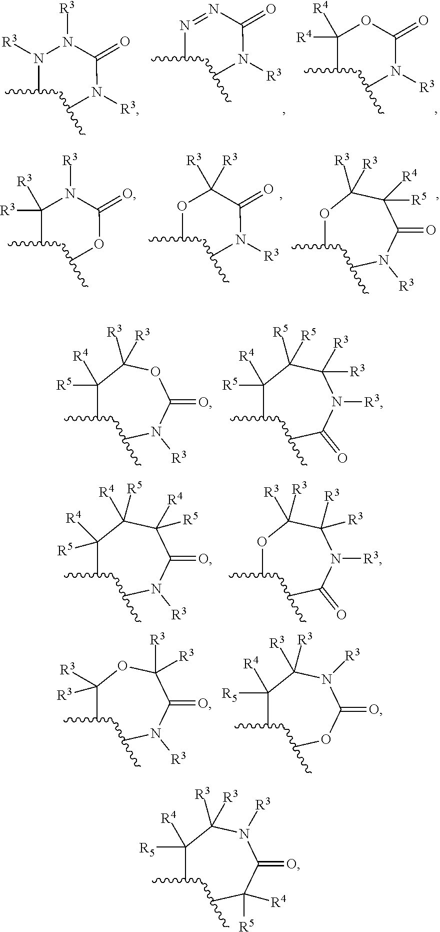

where R³ is as defined herein, R⁴ is a hydrogen atom, halogen atom, or alkyl group, and R⁵ is a hydrogen atom, halogen atom, alkyl group, or an alkoxy group.

In an embodiment, the compound has the following structure:

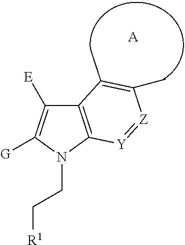

where E and G are replaced by the atoms of the following structures to form a ring:

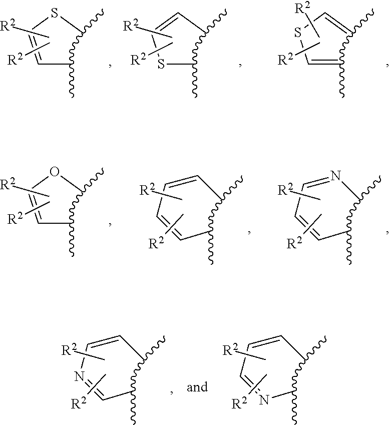

which can be optionally substituted with 0, 1, or 2 R² groups and R¹, Y, Z, and the A ring are as defined herein. In certain embodiments, the double bond between E and G is a single bond. For example, when E and G are replaced by

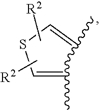

the bond between E and G is a single bond.

In an embodiment, the compound has the following structure:

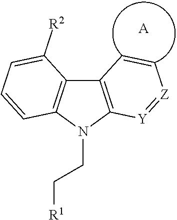

where ring A, R¹, R², Y, and Z are as defined herein.

In an embodiment, the compound has the following structure:

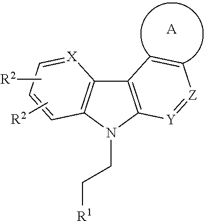

where X is a carbon or nitrogen atom and ring A, R¹, R², Y, and Z are as defined herein.

In an embodiment, the compound has the following structure:

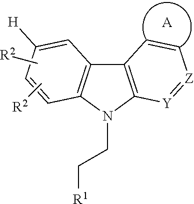

where X is a carbon or nitrogen atom and ring A, R¹, R², Y, and Z are as defined herein.

In an embodiment, the compound has the following structure:

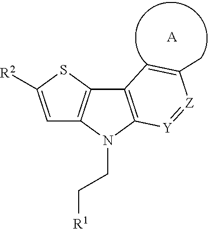

where ring A, R¹, R², Y, and Z are as defined herein.

In an embodiment, the compound has the following structure:

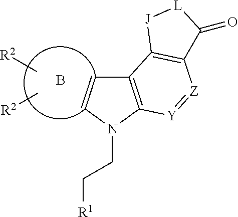

where J is an oxygen atom, —C(R⁴)₂, or —NR³, L is —C(R⁴)₂ or —NR³ and ring B, R¹, R², R³, R⁴, Y, and Z are as defined herein.

In an embodiment, the compound has the following structure:

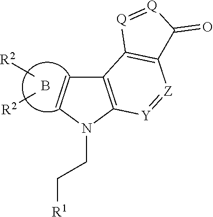

where each Q is independently —C(R³) or a nitrogen atom and ring B, R¹, R², R³, R⁴, Y and Z are as defined herein.

In an embodiment, the compound has the following structure:

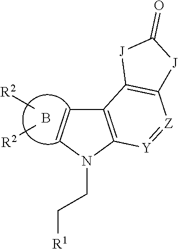

where each J is independently an oxygen atom, —C(R⁴)₂, or —NR³ and at most one J is an oxygen atom and ring B, R¹, R², R³, R⁴, Y and Z are as defined herein.

In an embodiment, the compound has the following structure:

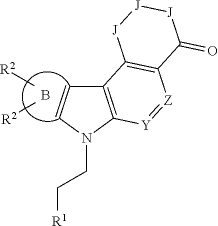

where each J is independently an oxygen atom, —C(R⁴)₂, or —NR³ and at most one J is an oxygen atom and ring B, R¹, R², R³, R⁴, Y and Z are as defined herein.

In an embodiment, the compound has the following structure:

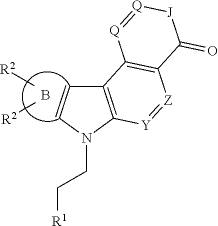

where each Q is independently —CR³ or a nitrogen atom, J is an oxygen atom, —C(R⁴)₂, or —NR³, and at least one Q is —CR³ and ring B, R¹, R², R³, R⁴, Y, and Z are as defined herein.

In an embodiment, the compound has the following structure:

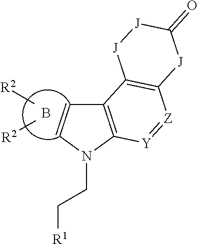

where each J is independently an oxygen atom, —C(R⁴)₂, or —NR³ and at most one J is an oxygen atom and ring B, R¹, R², R³, R⁴, Y and Z are as defined herein.

In an embodiment, the compound has the following structure:

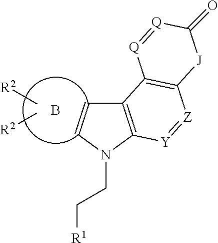

where each Q is independently —CR³ or a nitrogen atom, J is an oxygen atom, —C(R⁴)₂, or —NR³ and ring B, R¹, R², R³, R⁴, Y and Z are as defined herein.

In an embodiment, the compound has the following structure:

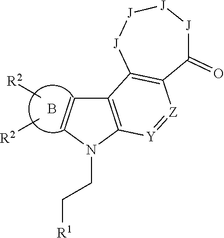

where each J is independently an oxygen atom, —C(R⁴)₂, or —NR³, at least one J is —C(R⁴)₂ and at most one J is an oxygen atom and ring B, R¹, R², R³, R⁴, Y and Z are as defined herein.

In an embodiment, the compound has the following structure:

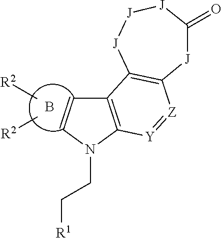

where J is an oxygen atom, —C(R⁴)₂, or —NR³, at least one J is —C(R⁴)₂ and at most one J is an oxygen atom and ring B, R¹, R², R³, R⁴, Y and Z are as defined herein.

In an embodiment, the compound has the following structure:

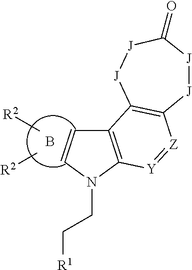

where J is an oxygen atom, —C(R⁴)₂, or —NR³, at most one J is an oxygen atom and ring B, R¹, R², R³, R⁴, Y, and Z are as defined herein.

In an embodiment, the compound has the following structure:

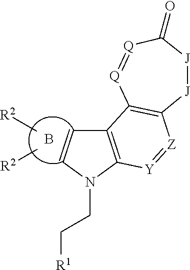

where Q is —CR³ or a nitrogen atom, J is an oxygen atom, —C(R⁴)₂, or —NR³, at most one J is an oxygen atom and ring B, R¹, R², R³, R⁴, Y and Z are as defined herein.

In an aspect, the present disclosure provides a method for inhibiting the growth of AR positive or negative cancer cells in an individual diagnosed with or suspected of having AR positive or negative cancer comprising administering to the individual a composition comprising a compound of the present disclosure.

### BRIEF DESCRIPTION OF THE FIGURES

FIGS. 1A-F. Cytotoxicity data for examples of the compounds.

FIG. 2. Metabolic stability of examples of the compounds in the presence of mouse hepatocytes. a) Half-life was calculated as t_{1/2}=0.693/k, where k is the elimination rate constant in the equation describing first order decay (Ct=C0·e^{-kt}), and C₁ and C₀ are the peak area ratios at time t and time 0, respectively. Data points were fitted to a first-order decay model by non-linear regression, using GraphPad Prism (version 5.04 or higher) without weighting or any user intervention. When the percent remaining was >50% at the longest incubation time or <50% at the shortest incubation time, the half-life is expressed as >the longest incubation time or <the shortest incubation time, respectively, and the calculated half-life is given in parentheses. b) Intrinsic clearance (C_{Lint}) was calculated based on C_{Lint}=k/P, where k is the elimination rate constant and P is the protein concentration in the incubation. NF-peak not found.

FIG. 3. Comparison of microsomal stability of examples of the compounds in the presence of mouse hepatocytes (HS) and mouse liver microsomes (MS).

FIG. 4. Pharmacokinetic profiles for examples of the compounds. The table shows concentrations of the compounds (μg) per ml of plasma at different time points after administration of the indicated dose IP or IV. Below is a graphical view of the same data.

FIG. 5. Dynamic of weight of mice treated with 5 daily IV or IP doses of PLA1125 or PLA1079.

FIG. 6. Growth of CWR22R tumors in SCID mice treated with 5 daily IV or IP doses of vehicle or PLA1125 or PLA1079. Data shown as fold of increase of tumor volume comparing with the day of start of treatment.

FIG. 7. Concentrations of PLA1079 and PLA1125 in tumors of mice treated with 5 daily doses of the drugs. Tumors were collected 24 hours after last administration.

FIG. 8. Pharmacokinetic (PK) data for PLA1098.

FIG. 9. PK data of PLA1148. Data from the mouse injected twice are shown in red.

FIG. 10. Scheme and summary of PLA1098 pilot efficacy testing. On the top is the scheme of drug administration and samples collection. First mouse was euthanized on day 3 and drug concentration was measured in plasma, liver and two tumors. Data are shown near first red arrow. Other 4 mice were euthanized on day 12. Plots demonstrate curves of individual tumor growth in control and PLA1098 group mice. Bar diagram shows average weight of excised tumors from control and PLA1098 treated mice+/−standard deviation.

FIG. 11. Expression of Caveolin 1 gene in a panel of breast cancer cell lines with different c52 sensitivity.

FIG. 12. Expression of Caveolin1 in sensitive versus resistant cells. Caveolin1 is expressed in resistant MDA MB 231 cells, but not in sensitive MCF7 cells. Treatment of cells with c52 or PLA1079 did not change levels of Caveolin1 expression. Gapdh was used as a loading control, overexpression of p21 in sensitive cells upon treatment with the compounds confirmed the activity of used compounds.

FIG. 13. c52 causes a DNA-damage-response type of p53 activation in sensitive, but not resistant cells. Results of Western Blot analysis. Sensitive (MCF7 and CWR22r) and resistant (NKE) cells were treated with 1 uM of c52 for indicated time-periods. In sensitive, but not resistant cells c52 caused elevation of p53 amount, more than that c52 treatment induced phosphorylation of p53 by Serines 15 and 329-hallmarks of DNA damage response activation.

FIG. 14. c52 causes replication stress in sensitive, but not resistant cells. A. Immuno-histological staining with specific antibodies to RPA70 and XRCC1 (proteins, accumulating at sites of stalled replication forks) in sensitive (MCF7) or resistant (MDA MB 231) cells treated with c52 versus control untreated ones. Clear formation of loci of both proteins might be observed in treated sensitive cells. B. c52 treatment abrogates incorporation of Edu in sensitive MCF7 cells, indicating stall of replication.

FIG. 15. c52 induces accumulation of Mdm2 in sensitive CWR22r cells. Results of Western Blot analysis: CWR22r cells were treated with luM c52 for indicated time intervals.

FIG. 16 Inhibition of p53 activity by expression of its dominant-negative forms does not abrogate c52-caused degradation of AR level in CWR22r or MCF7 cells. A. Though p53 activity is inhibited by introduction of its dominant-negative mutant we still observe decrease of AR upon c52 treatment in CWR22r cells. B. In MCF7 cells with functionaly inactive p53 (R175H, GSE56) Mdm2 is no longer overexpressed upon treatment with PLA1079 or p53 activator CBL 137. Still, AR is being degraded upon this treatment.

FIG. 17. Summary of PK data of tested PLA compounds.

FIG. 18. Scheme and data of pilot efficacy testing of the compound PLA1163. Curves which end earlier than other indicate tumors which were collected to measure intra-tumor drug concentration at the end of treatment.

FIG. 19. Weight of CWR22R tumors excised from mice at the end of experiment (day 12 after start of treatment). Bars—mean of tumor weight within each group (n=5-10) , error bars—standard deviation.

FIG. 20. Plot of plasma concentrations of different PLA compounds at different time points after single IV administration of 50 mg/kg.

FIG. 21. Toxicity of PLA1055 to CWR22R cells in vitro depending on time of incubation. PLA1055 was added to CWR22R in full range of concentrations for the periods of time shown on the right. After that drug containing media were changed for drug free and survival of cells was detected at 72 hours after start of treatment using Alamar Blue assay (Promega).

FIG. 22. Activation of Caspase 3/7 by c52 vs Doxorubicin (Dox) in sensitive and resistant cells with a different status of p53. Cells were incubated with c52 or doxorubicin in indicated concentrations for 16 hours. After that substrate to Caspases 3/7 was added and activity of caspases 3/7 (which would indicate apoptosis occurrence) was estimated by measuring the substrate cleavage.

FIG. 23. Ectopic Caveolin1 did not save sensitive cells from sensitivity to c52. A. Results of WB analysis: AR is being decreased and p53 activated in Caveolinl expressing cells, same as in regular ones. B. MCF7 cells, introduced with Caveolinl expressing construct, remain sensitive to c52.

FIG. 24. DARTS (Drug Affinity Responsive Target Stability) assay was performed using c52 and PLA1118. According to this assay these compounds are capable of protecting presumable target protein from protease degradation. Protein lysates from sensitive CWR22r cells were incubated with or without the drug and subsequently digested with the indicated concentration of pronase. A. c52 was used (lane T-treated, M-marker) with indicated concentrations of pronase; ability of c52 to protect its target from protease cleavage was judged based on presence of protein band in the treated lane vs the untreated (UT) control. B. Additional compounds PLA1098 (an active analogue of c52) and PLA1118 (inactive analog) were used to confirm the results. The protein band appeared in c52 and PLA1098 (shown by arrows) as opposed to the untreated and faintly in PLA1118 samples indicating that both of the active compounds protected their targets from degradation.

FIG. 25. Efficacy (A) and Stability (B) of biotinylated PLA1200 and PLA1201 compounds.

### DETAILED DESCRIPTION OF THE DISCLOSURE

The present disclosure provides carbazole compounds and carbazole-like compounds (e.g., pyridoindole and pyrrolodipyridine compounds). The compounds can selectively kill cancer cells.

In an aspect, the present disclosure provides heterocyclic compounds having the following structure:

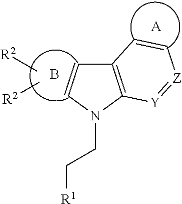

where R¹ is selected from the group consisting of a hydrogen atom, CH₃, CH₂F, CHF₂ and CF₃; R² is independently at each occurrence a hydrogen atom, halogen atom, —CN, —OH, substituted or unsubstituted alkyl, substituted or unsubstituted alkoxy group, —C(═O)N(R³)₂, —N(R³)₂, ketone, substituted or unsubstituted cycloalkyl group, or substituted or unsubstituted heterocycloalkyl group; Y and Z are independently a carbon or nitrogen atom; ring A is a substituted or unsubstituted 5 to 7 membered carbocyclic or heterocyclic ring; ring B is a substituted or unsubstituted 5 to 6 membered aryl or heteroaryl ring; and R³ is a hydrogen atom or substituted or unsubstituted alkyl. The compound has 0-2 R² groups.

As used herein, the term “alkyl group” refers to branched or unbranched hydrocarbons. Examples of such alkyl groups include methyl groups, ethyl groups, propyl groups, butyl groups, isopropyl groups, tert-butyl groups, and the like. For example, the alkyl group can be a C₁ to C₄ alkyl group, including all integer numbers of carbons and ranges of numbers of carbons therebetween. Alkyl groups can be substituted with various other functional groups. For example, the alkyl groups can be substituted with groups such as, for example, amines (acyclic and cyclic)

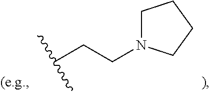 [SMILES: *CCN1CCCC1]

alcohol groups

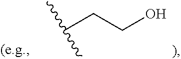 [SMILES: *CCO]

ether groups

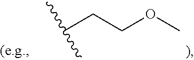 [SMILES: *CCOC]

and halogen atoms.

As used herein, the term “halogen atom” refers to a fluorine, chlorine, bromine, or iodine atom.

As used herein, the term “nitrile” refers to the following structure:

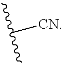 [SMILES: *C#N]

As used herein, the term “carbonyl” refers to the following structure:

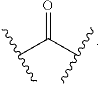 [SMILES: *C(*)=O]

Carbonyl groups are known by those skilled in the art. Ketones and amides are examples of “carbonyl groups.” As used herein, the term “ketone” refers to the following structure:

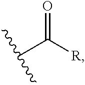

where R is an alkyl group as described herein. Where R is a methyl group, this structure is referred to as an “acyl” group. As used herein, the term “amide” refers to the following structure:

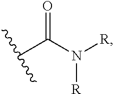

where each R is independently a hydrogen atom or alkyl group. Thus, the amide can be a primary, secondary, or tertiary amide.

As used herein, the term “alkoxy” groups refers to the following structure:

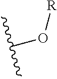

where R is an alkyl group as described herein.

As used herein, the term “aryl ring” refers to an aromatic carbocyclic group of 6 carbon atoms having a single ring (e.g., phenyl). The aryl group is substituted with 0, 1, or 2 R² groups as described herein.

As used herein, the term “heteroaryl ring” refers to an aromatic cyclic ring (i.e., fully unsaturated) having 1, 2, 3, or 4 carbon atoms and 1, 2, 3, or 4 heteroatoms selected from oxygen, nitrogen, and sulfur. Examples of heteroaryl rings include thiophene, furan, and pyridine. The heteroaryl group is substituted with 0, 1, or 2 R² groups as described herein.

As used herein, the term “cycloalkyl group” refers to a to a saturated or partially unsaturated carbocyclic group (not aromatic) of from 4 carbons to 11 carbons having a single cyclic ring or multiple condensed rings. For example, the cycloalkyl groups can be cyclobutane, cyclopentane, cyclohexane, cyclohexene, cycloheptane, cycloheptene, bicyclo[2.1.1]hexane, bicyclo[2.2.1]heptane, bicyclo[2.2.2]octane, bicyclo[3.3.0]octane, bicyclo[4.4.0]octane, and the like. Cycloalkyl also includes carbocyclic groups to which is fused an aryl or heteroaryl ring, for example indane and tetrahydronaphthalene. The cycloalkyl groups can be unsubstituted or substituted with groups such as, for example, alkyl, carbonyl, or halogen.

As used herein, the term “heterocycloalkyl group” refers to a saturated or partially unsaturated group having a single cyclic ring or multiple condensed having from 2 to 11 carbon atoms and 1 to 5 heteroatoms, selected from nitrogen, oxygen, sulfur, and combinations thereof. For example, the heterocycloalkyl groups can be, for example, dihydrofuran, tetrahydrofuran, pyrrolidine, dihydropyran, tetrahydropyran, 1,3 dioxane, 1,4-dioxane, dihydropyridinone, piperidine, piperazine, morpholine, thiomorpholine, urazole, 2-aza-bicyclo[2.2.2]oct-5-ane-3-one, and the like. Heteroccycloalkyl also includes heterocyclic groups to which is fused an aryl or heteroaryl ring, for example tetrahydroisoquinoline or indoline. The heterocycloalkyl groups can be unsubstituted or substituted with groups such as, for example, alkyl, carbonyl, or halogen.

As used herein, the term “heterocycle” or “heterocyclic ring” refers to a cyclic compound having a ring where at least one or more of the atoms forming the ring is a heteroatom (e.g., oxygen, nitrogen, sulfur, etc.). The heterocyclic ring can be aromatic or nonaromatic, and include compounds that are saturated, partially unsaturated, and fully unsaturated. Examples of such groups include furan, thiophene, oxazole, isoxazole, thiazole, oxadiazole, thiadiazole, triazole, tetrazole, oxazoline, lactam, lactone, dihydrofuran, tetrahydrofuran, furanone, oxazolone, pyridinone, pyrimidinone, dihydropyridazine, pyranone, oxazinone, and the like. For example, the heterocyclic ring can be a 5 to 7 membered ring containing a number of carbon atoms ranging between 2 and 6 and a number of heteroatoms ranging between 1 and 4. The heterocyclic ring can be unsubstituted or substituted with groups such as, for example, alkyl, carbonyl, or halogen.

As used herein, the term “carbocyclic ring” refers to a cyclic compound having a ring where all of the atoms forming the ring are carbon atoms. The carbocyclic ring can be aromatic or nonaromatic, and include compounds that are saturated and partially unsaturated, and fully unsaturated. Examples of such groups include cyclopentane, cyclopentene, cyclohexane, cyclohexene, cyclohexanone, cyclopentanone, cyclopentanol, indane, indanone, phenyl, naphthyl and the like. For example, the carbocyclic ring is a C₅ to C₇ carbocyclic ring, including all integer numbers of carbons and ranges of numbers of carbons therebetween. The carbocyclic ring can be unsubstituted or substituted with groups such as, for example, alkyl, carbonyl, or halogen.

In an embodiment, ring A is a 5 to 7 membered ring, for example a cyclic ketone, lactam, lactone, furanone, oxazolone, dioxolane, pyridinone, pyrimidinone, pyridazinone, dihydropyridazine, pyranone, or oxazinone. The 5 to 7 membered ring can be substituted with alkyl group(s) on carbon and/or nitrogen. In an embodiment, the compound has the following structure:

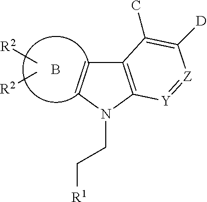

where C and D are replaced by the atoms of the following structures to form a ring:

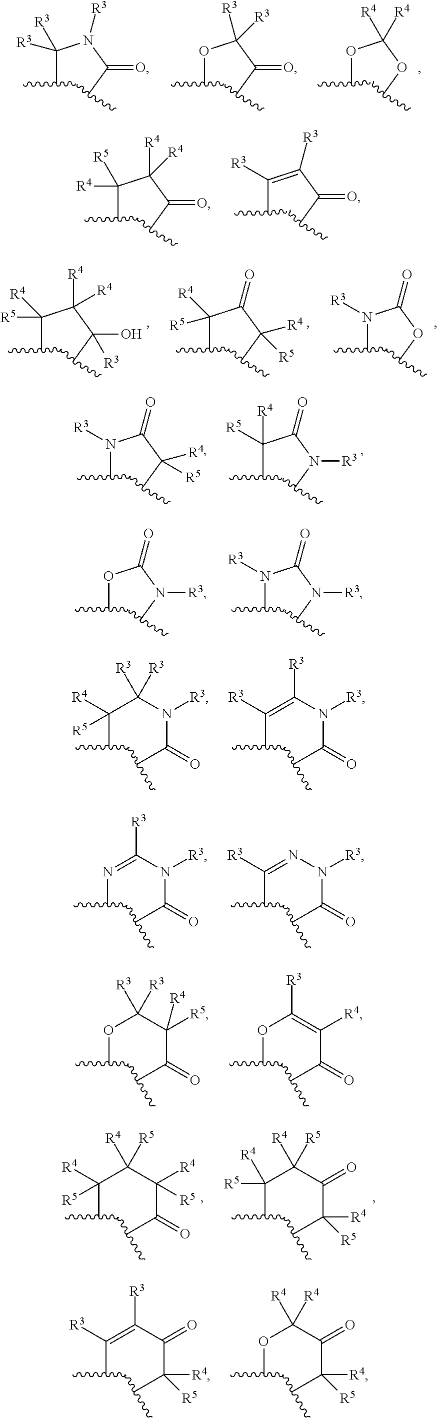

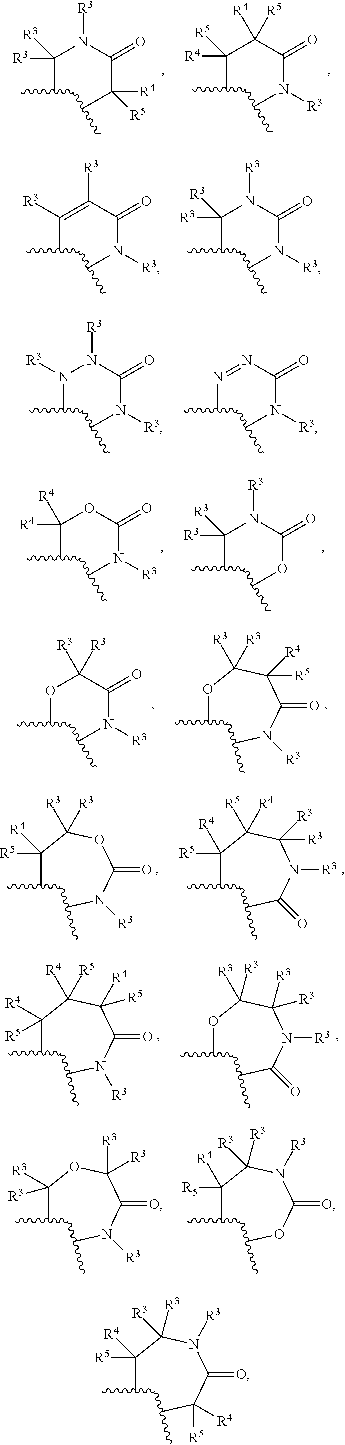

where R³ is as defined herein, R⁴ is a hydrogen atom, halogen atom, or alkyl group, and R⁵ is a hydrogen atom, halogen atom, alkyl group, or an alkoxy group.

In an embodiment, the compound has the following structure:

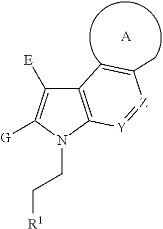

where E and G are replaced by the atoms of the following structures to form a ring:

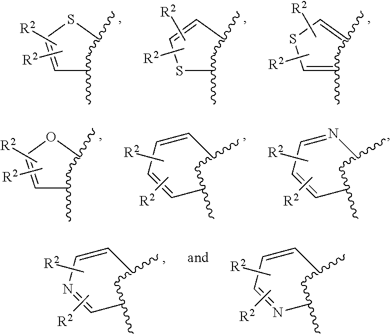

which can be optionally substituted with 0, 1, or 2 R² groups and R¹, Y, Z, and the A ring are as defined herein. In certain embodiments, the double bond between E and G is a single bond. For example, when E and G are replaced by

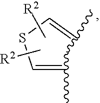

the bond between E and G is a single bond.

In various embodiments, the compound is a salt (e.g., a hydrochloride salt, an N-oxide), a partial salt, a hydrate, a polymorph, a stereoisomer or a mixture thereof. The compounds can have stereoisomers. For example, the compound is present as a racemic mixture, a single enantiomer, a single diastereomer, mixture of enantiomers, or mixture of diastereomers.

In an embodiment, the compound has the following structure:

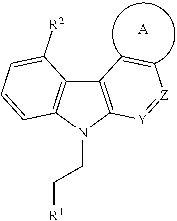

where ring A, R¹, R², Y, and Z are as defined herein.

In an embodiment, the compound has the following structure:

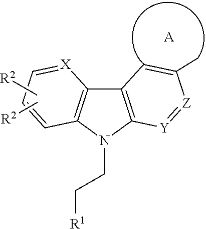

where X is a carbon or nitrogen atom and ring A, R¹, R², Y, and Z are as defined herein.

In an embodiment, the compound has the following structure:

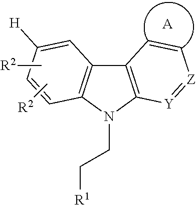

where X is a carbon or nitrogen atom and ring A, R¹, R², Y, and Z are as defined herein.

In an embodiment, the compound has the following structure:

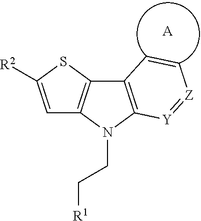

where ring A, R¹, R², Y, and Z are as defined herein.

In an embodiment, the compound has the following structure:

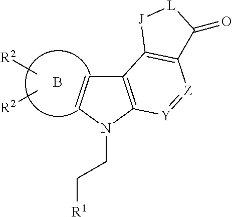

where J is an oxygen atom, —C(R⁴)₂, or —NR³, L is —C(R⁴)₂ or —NR³ and ring B, R¹, R², R³, R⁴, Y, and Z are as defined herein.

In an embodiment, the compound has the following structure:

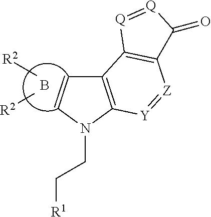

where each Q is independently —C(R) or a nitrogen atom and ring B, R¹, R², R³, R⁴, Y and Z are as defined herein.

In an embodiment, the compound has the following structure:

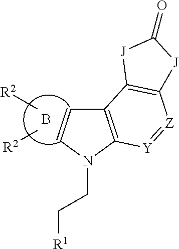

where each J is independently an oxygen atom, —C(R⁴)₂, or —NR³ and at most one J is an oxygen atom and ring B, R¹, R², R³, R⁴, Y and Z are as defined herein.

In an embodiment, the compound has the following structure:

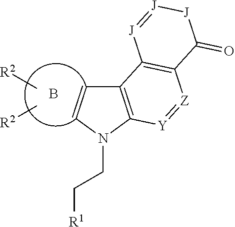

where each J is independently an oxygen atom, —C(R⁴)₂, or —NR³ and at most one J is an oxygen atom and ring B, R¹, R², R³, R⁴, Y and Z are as defined herein.

In an embodiment, the compound has the following structure:

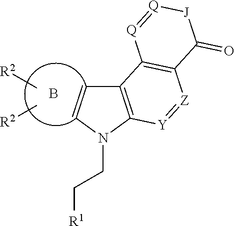

where each Q is independently —CR³ or a nitrogen atom, J is an oxygen atom, —C(R⁴)₂, or —NR³, and at least one Q is —CR³ and ring B, R¹, R², R³, R⁴, Y, and Z are as defined herein.

In an embodiment, the compound has the following structure:

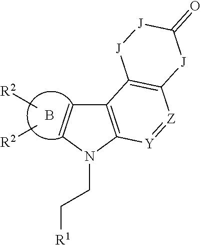

where each J is independently an oxygen atom, —C(R⁴)₂, or —NR³ and at most one J is an oxygen atom and ring B, R¹, R², R³, R⁴, Y and Z are as defined herein.

In an embodiment, the compound has the following structure:

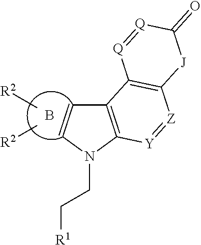

where each Q is independently —CR³ or a nitrogen atom, J is an oxygen atom, —C(R⁴)₂, or —NR³ and ring B, R¹, R², R³, R⁴, Y and Z are as defined herein.

In an embodiment, the compound has the following structure:

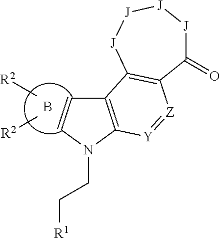

where each J is independently an oxygen atom, —C(R⁴)₂, or —NR³, at least one J is -C(R⁴)₂ and at most one J is an oxygen atom and ring B, R¹, R², R³, R⁴, Y and Z are as defined herein.

In an embodiment, the compound has the following structure:

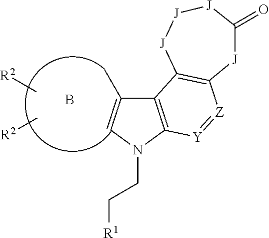

where J is an oxygen atom, —C(R⁴)₂, or —NR³, at least one J is —C(R⁴)₂ and at most one J is an oxygen atom and ring B, R¹, R², R³, R⁴, Y and Z are as defined herein.

In an embodiment, the compound has the following structure:

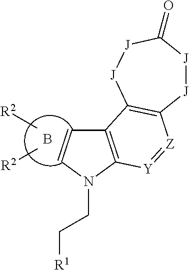

where J is an oxygen atom, —C(R⁴)₂, or —NR³, at most one J is an oxygen atom and ring B, R¹, R², R³, R⁴, Y, and Z are as defined herein.

In an embodiment, the compound has the following structure:

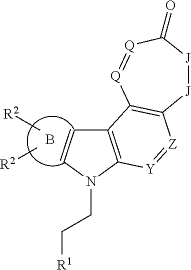

where Q is —CR³ or a nitrogen atom, J is an oxygen atom, —C(R⁴)₂, or —NR³, at most one J is an oxygen atom and ring B, R¹, R², R³, R⁴, Y and Z are as defined herein.

In an embodiment, the compound has one of the following structures:

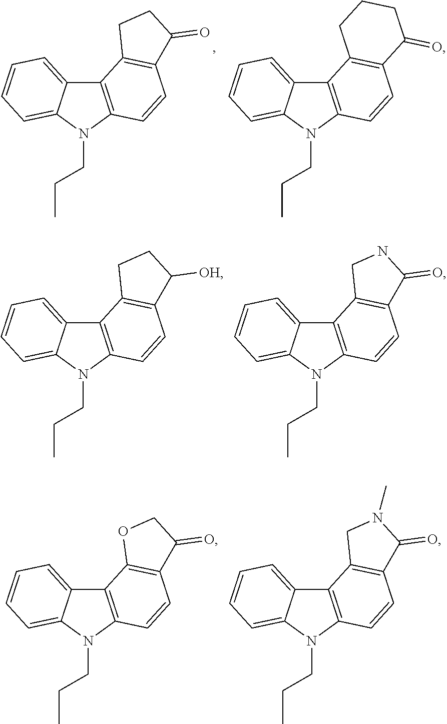

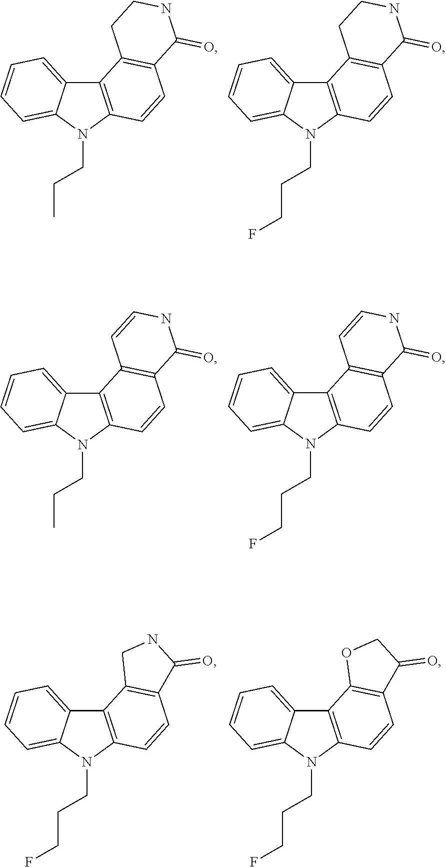

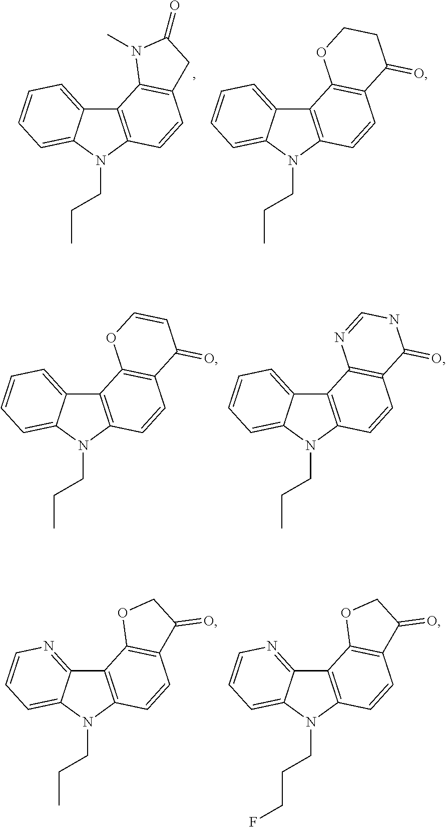

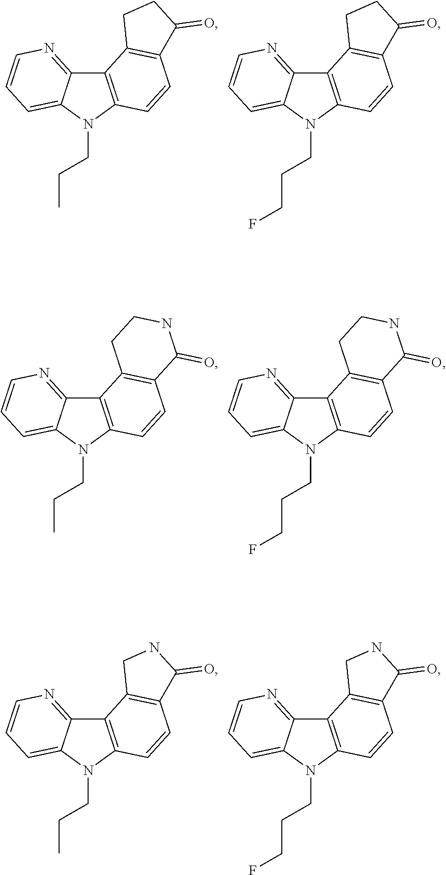

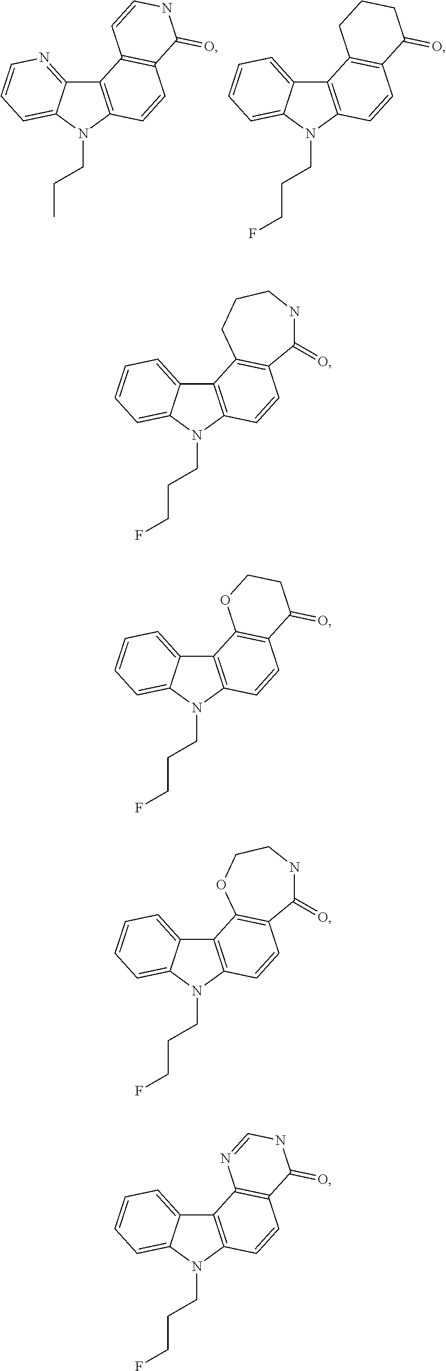

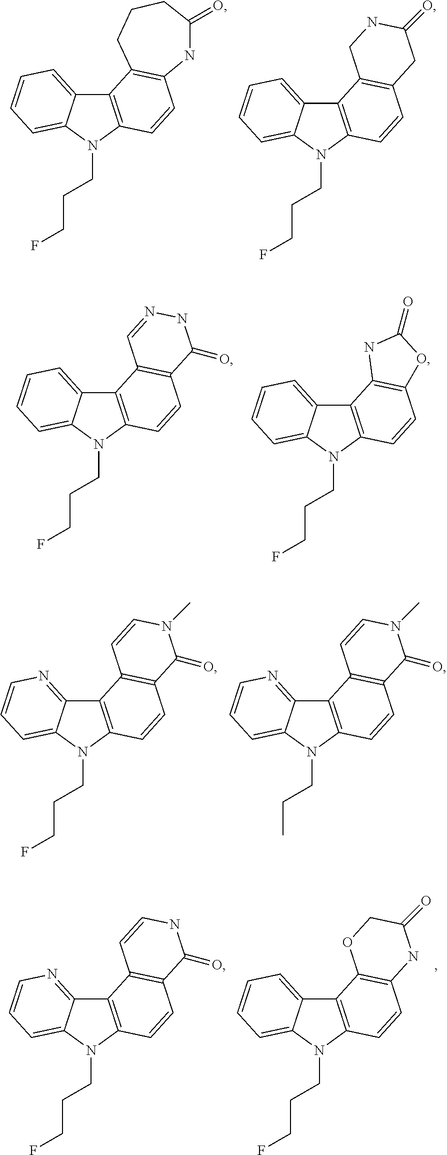

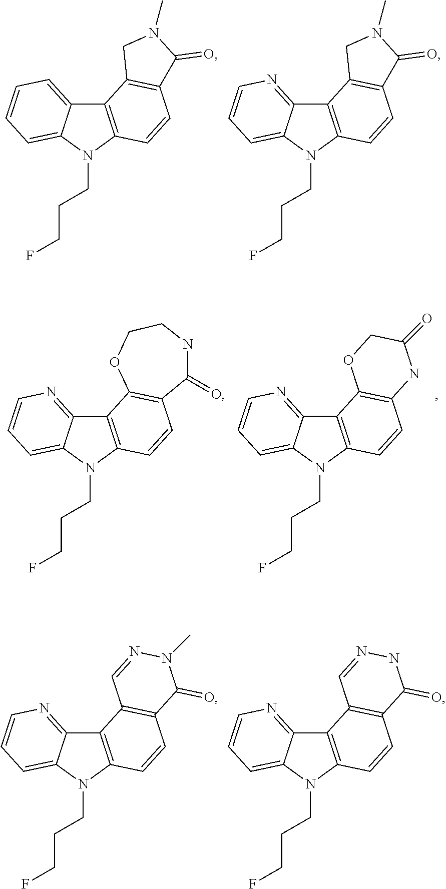

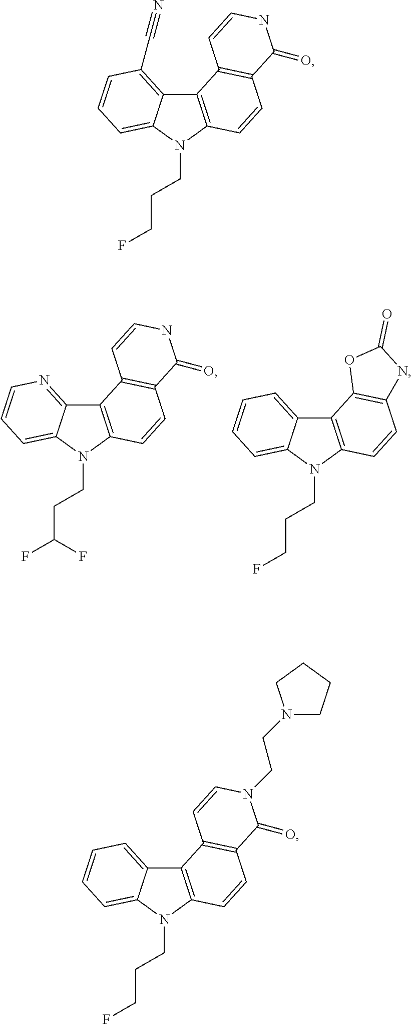

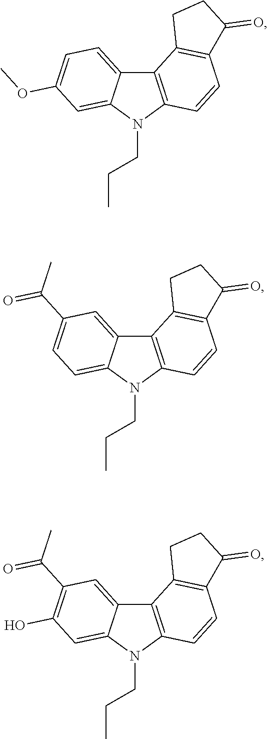 [SMILES: CCCn1c2cc(OC)ccc2c2c3c(ccc21)C(=O)CC3 ; CCCn1c2ccc(C(C)=O)cc2c2c3c(ccc21)C(=O)CC3 ; CCCn1c2cc(O)c(C(C)=O)cc2c2c3c(ccc21)C(=O)CC3]

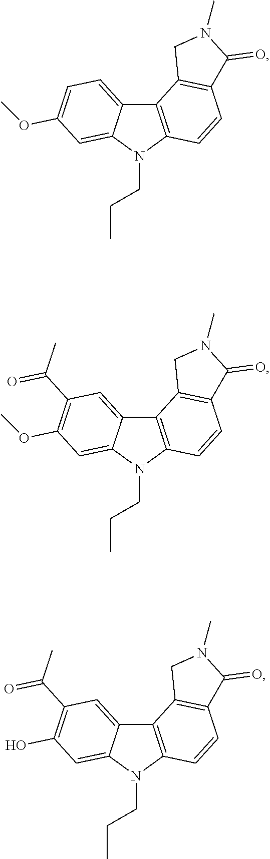 [SMILES: CCCn1c2cc(OC)ccc2c2c3c(ccc21)C(=O)N(C)C3 ; CCCn1c2cc(OC)c(C(C)=O)cc2c2c3c(ccc21)C(=O)N(C)C3 ; CCCn1c2cc(O)c(C(C)=O)cc2c2c3c(ccc21)C(=O)N(C)C3]

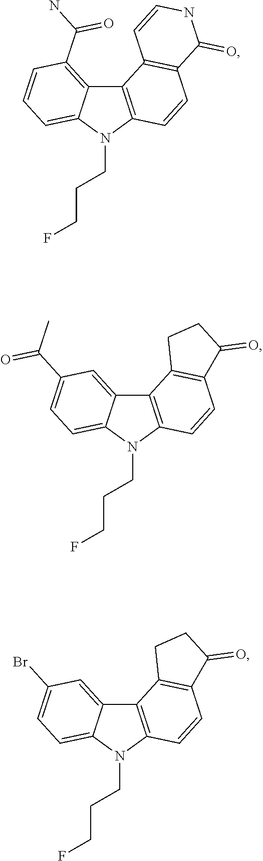 [SMILES: NC(=O)c1cccc2c1c1c3cc[nH]c(=O)c3ccc1n2CCCF ; CC(=O)c1ccc2c(c1)c1c3c(ccc1n2CCCF)C(=O)CC3 ; O=C1CCc2c1ccc1c2c2cc(Br)ccc2n1CCCF]

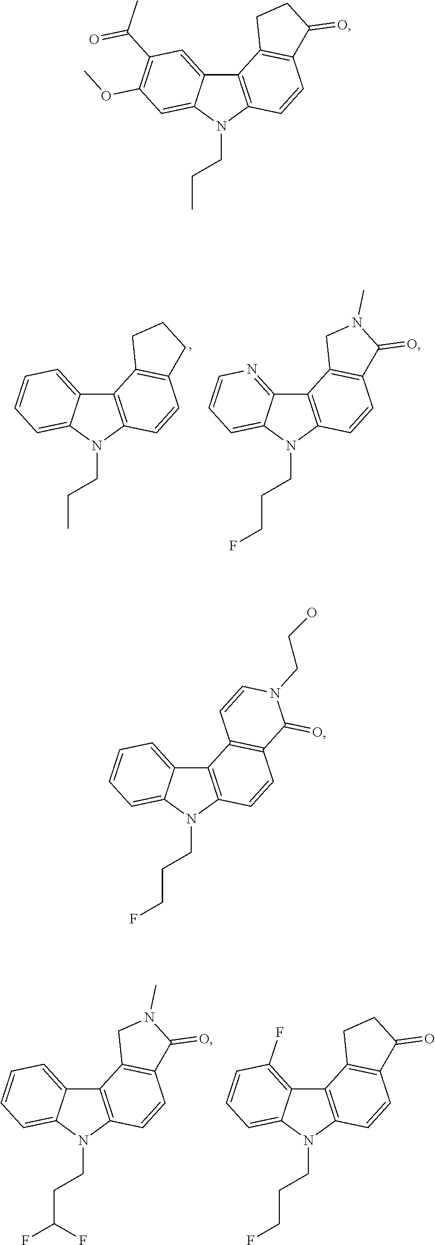

Non-limiting examples of general methods for the preparation of the compounds of the present disclosure are provided in the following schemes:

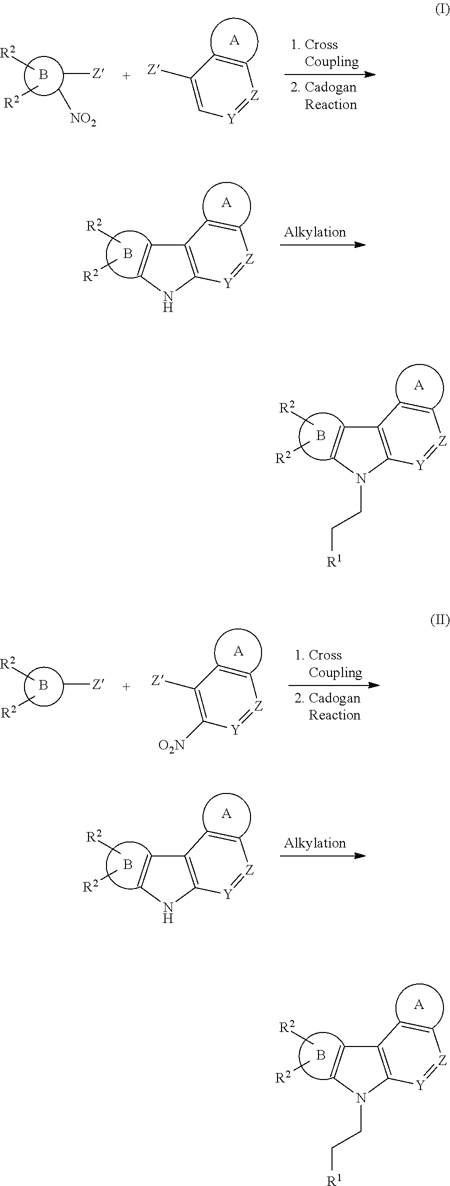

where each Z′, independently is a halogen, a trifluoromethanesulfonate, a trialkyltin, a boronic acid, or boronic ester as long as one coupling partner Z′ is a halogen and the other coupling partner Z′ is not a halogen. Ring A, ring B, R¹, R², Y, and Z are as defined herein. The determination of suitable reaction conditions for cross coupling, the Cadogan reaction, alkylation, and other functional group transformations (e.g., metal complex, base, reagents, solvent, reaction time, and reaction temperature) are within the purview of one having skill in the art. In certain circumstances, it may be necessary to form the heterocycles of the present disclosure by well-established condensation reactions. To assemble the coupling partners or further functionalize the aromatic components of the present disclosure it may be necessary to use of electrophilic aromatic substitution reactions, nucleophilic aromatic substitution reactions, anion chemistry, and the like. Other oxidation state and functional groups manipulations are within the purview of one having skill in the art.

More specific, non-limiting, examples of methods to synthesize compounds of the present are illustrated in the examples that follow.

In an aspect, the present disclosure provides a composition comprising at least one compound of the disclosure. Compositions comprising at least one compound of the disclosure include, for example, pharmaceutical preparations.

Compositions comprising a compound of the disclosure and a pharmaceutical carrier can be prepared at a patient's bedside, or by a pharmaceutical manufacture. In either case, the compositions or their ingredient can be provided in any suitable container, such as a sealed sterile vial or ampoule, and may be further packaged to include instruction documents for use by a pharmacist, physician or other health care provider. The compositions can be provided as a liquid, or as a lyophilized or powder form that can be reconstituted if necessary when ready for use. In particular, the compositions can be provided in combination with any suitable delivery form or vehicle, examples of which include, for example, liquids, caplets, capsules, tablets, inhalants or aerosol, etc. The delivery devices may comprise components that facilitate release of the pharmaceutical agents over certain time periods and/or intervals, and can include compositions that enhance delivery of the pharmaceuticals, such as nanoparticle, microsphere or liposome formualtions, a vareity of which are known in the art and are commercially available. Further, each composition described herein can comprise one or more pharmaceutical agents.

The compositions described herein can include one or more standard pharmaceutically acceptable carriers. Some examples of pharmaceutically acceptable carriers can be found in: Remington: The Science and Practice of Pharmacy (2005) 21st Edition, Philadelphia, Pa. Lippincott Williams & Wilkins.

Various methods known to those skilled in the art can be used to introduce the compositions of the disclosure to an individual. These methods, for example, intravenous, intratumeral, intramuscular, intracranial, intrathecal, intradermal, subcutaneous, vaginal, rectal, and oral routes. The dose of the composition comprising a compound of the disclosure and a pharmaceutical agent will necessarily be dependent upon the needs of the individual to whom the composition of the disclosure is to be administered. These factors include, for example, the weight, age, sex, medical history, and nature and stage of the disease for which a therapeutic or prophylactic effect is desired. The compositions can be used in conjunction with any other conventional treatment modality designed to improve the disorder for which a desired therapeutic or prophylactic effect is intended, non-limiting examples of which include surgical interventions and radiation therapies. The compositions can be administered once, or over a series of administrations at various intervals determined using ordinary skill in the art, and given the benefit of the present disclosure.

The AR positive or negative cancer cells referred to herein are cancer cells that express a detectable amount of AR protein. “Androgen receptor” (and thus its abbreviation “AR”) is a term well known to those skilled in the art and is used herein to refer to AR protein expressed by human cancer cells, including all isoforms and allelic variants of human AR protein.

In an embodiment, AR positive or negative cancer cells, the growth of which can be inhibited in an individual by practicing the method of the disclosure, are cells that express AR that is specifically recognized by any type of anti-human AR antibody. Anti-human AR antibodies are commercially available. In an embodiment, AR positive or negative cancer cells, the growth of which can be inhibited in an individual by practicing the method of the disclosure, are cells that express AR that can be specifically recognized by the anti-human AR antibody available from BD PharMingen, San Diego, Calif., under catalog number #554225. In an embodiment, a detectable amount of AR protein is an amount of AR protein that can be detected by a Western blot.

In an embodiment, AR positive or negative cancer cells are cells that express AR having the amino acid sequence for GenBank accession no. P10275, Sep. 1, 2009 entry, which is incorporated herein by reference. In alternative embodiments, AR positive or negative cancer cells are cells that express AR having an amino acid sequence that is between 70%-99%, inclusive, and including all integers there between, homologous to the amino acid sequence provided for GenBank accession no. P 10275, Sep. 1, 2009. The AR positive or negative cells can by cancer cells that express such an AR having any of such sequences, wherein the AR is detectable by Western blot.

In another embodiment, the AR positive or negative cells are breast cancer cells. The breast cancer cells may be any type of breast cancer cells, provided they are AR positive or negative. The breast cancer cells may be any of ER-, PR-, Her2-, or combinations thereof.

The inhibition of growth of the AR positive or negative cancer cells may be partial inhibition or complete inhibition. Eradication of some or all AR positive or negative cancer cells from an individual is considered to be a type of inhibition of growth of the AR positive or negative cancer cells.

In an aspect, the present disclosure provides a method for treating various androgen receptor positive or negative cancer cells using the compounds as described herein. In an embodiment, the type of cancer cells are selected from the group consisting of prostate cancer, breast cancer, and hepatocellular carcinoma (HCC), thyroid cancer, glioblastoma, or astrocytoma. In an embodiment, certain compounds are particularly useful against certain types of AR positive cancers. In an embodiment, certain compounds are particularly useful against certain types of AR negative cancers. In another embodiment, certain compounds are particularly useful against certain types of both AR positive and AR negative cancers.

In an aspect, the present disclosure provides a method for reducing the number of AR positive or negative cancer cells in a cell culture using the compounds as described herein.

In an aspect, the present disclosure provides a method for inhibiting the growth of AR positive or negative cancer cells in an individual. The method comprises administering to an individual diagnosed with or suspected of having AR positive or negative cancer a composition comprising a compound capable of inhibiting the growth of or killing AR positive or negative cancer cells. General structures of compounds suitable for use in the disclosure are depicted herein.

In an embodiment, the method of the disclosure comprise administering to an individual diagnosed with or suspected of having AR positive or negative cancer a compound as described herein. For example, the AR positive or negative cancer is prostate cancer, breast cancer, or hepatocellular carcinoma (HCC), thyroid cancer, glioblastoma, or astrocytoma.

In an embodiment of the disclosure, an individual can be identified as a candidate for treatment with a composition comprising an effective amount of a compound as described herein. The individual can be identified as such a candidate by obtaining a biological sample of cancerous tissue from the individual and determining whether or not the cancerous tissue expresses AR. Determining the cancerous tissue expresses AR is indicative that the individual is a candidate for the treatment. Determining that the tissue does not express a detectable amount of AR is indicative that the individual is not a candidate for the treatment. Determining whether the cancerous tissue expresses AR can be performed using any suitable technique, such as immunological techniques. In an embodiment, the disclosure includes transforming AR in a biological sample obtained from the individual into an AR-antibody complex, and detecting the AR-antibody complex using an immunodiagnostic device.

The following specific examples are to be construed as merely illustrative, and not limitative of the remainder of the disclosure in any way whatsoever.

All synthetic chemistry was performed in standard laboratory glassware unless indicated otherwise in the examples. Commercial reagents were used as received. Analytical LC/MS was performed on an Agilent 1200 system with a variable wavelength detector and Agilent 6140 single quadrupole mass spectrometer, alternating positive and negative ion scans. Retention times were determined from the extracted 220 nm UV chromatogram. ¹H NMR was performed on a Bruker DRX-400 at 400 MHz or a Bruker Avance DRX 500 at 500 MHz. For complicated splitting patterns, the apparent splitting is tabulated. Microwave reactions were performed in a Biotage Initiator using the instrument software to control heating time and pressure. Silica gel chromatography was performed manually, or with an Isco CombiFlash for gradient elutions.

Analytical LC/MS method A: HPLC column: Kinetex, 2.6 μm, C18, 50×2.1 mm, maintained at 40° C.HPLC Gradient: 1.0 mL/min, 95:5:0.1 water:acetonitrile:formic acid to 5:95:0.1 water:acetonitrile:formic acid in 2.0 min, maintaining for 0.5 min.Reported retention times are for method A unless indicated otherwise.

Analytical LC/MS method B was performed on a Shimadzu system with an attached API 165 single quadrupole mass spectrometer. Retention times were determined from the 220 nm chromatogram.HPLC column: Phenomenex, C18, 2.5 μm, 20×2 mm, maintained at 25° C.HPLC Gradient: 0.5 mL/min, 95:5:0.02 water:acetonitrile:CF₃COOH to 5:95:0.02 water:acetonitrile:CF₃COOH in 2.9 min, maintaining for 0.9 min.

### PREPARATIONS

### Preparation 1-1. 5-Bromo-3,4-dihydro-1H-quinolin-2-one

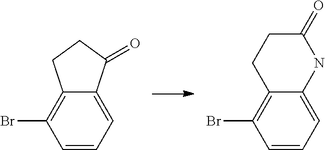 [SMILES: O=C1CCc2c(Br)cccc21 ; O=C1CCc2c(Br)cccc2N1]

A biphasic mixture of sodium azide (18.5 g, 284 mmol), sulfuric acid (18.8 M, 4.8 mL, 90 mmol), water (36 mL) and chloroform (144 mL) was stirred at 0° C. for 2.5 h. The layers were separated and the organic layer was dried over sodium sulfate and filtered. The filtrate was added to a solution of 4-bromoindan-1-one (12.0 g, 56.9 mmol) in chloroform (215 mL). To this solution was added sulfuric acid (18.8 M, 18.7 mL, 351.6 mmol) dropwise over 10 min. The reaction mixture was stirred at 45° C. for 4 h, then cooled to room temperature and stirred for 20 h. The mixture was poured onto ice (200 g) and neutralized by addition of 10% aqueous sodium hydroxide (50 mL). The layers were separated and the aqueous layer was extracted with chloroform (100 mL). The combined organic layers were dried over sodium sulfate and evaporated. The crude product was recrystallized from ethanol (55 mL) to give the title compound (8.65 g, 38.2 mmol, 67%) as an off-white powder. LCMS: 98%, Rt 1.290, ESMS m/z 226 (M+H)⁺.

### Preparation 2-1. 5-Bromo-3,4-dihydro-2H-isoquinolin-1-one

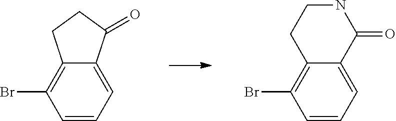 [SMILES: O=C1CCc2c(Br)cccc21 ; O=C1NCCc2c(Br)cccc21]

To a mixture of 4-bromoindan-1-one (4.00 g, 18.9 mmol) and methanesulfonic acid (20.2 mL, 310 mmol) in dichloromethane (180 mL) was added sodium azide (2.46 g, 37.9 mmol) at 0° C. The mixture was warmed to room temperature and stirred for 16 h. The reaction mixture was poured into 10% aqueous sodium hydroxide (200 mL) and extracted with dichloromethane (100 mL). The combined organic layers were dried over sodium sulfate and evaporated. The crude product was recrystallized from ethyl acetate (40 mL) to give the title compound (3.98 g, 17.6 mmol, 93%) as a white powder. LCMS: 96%, Rt 1.225, ESMS m/z 226 (M+H)⁻.

### Preparation 3-1. 8-Bromo-3H-quinazolin-4-one

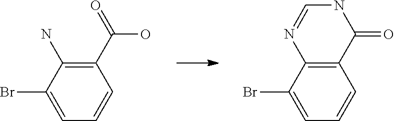 [SMILES: Nc1c(Br)cccc1C(=O)O ; O=c1[nH]cnc2c(Br)cccc12]

A solution of 2-amino-3-bromobenzoic acid (0.96, 4.44 mmol) in formamide (3 mL) was heated at 135° C. for 90 min, then at 175° C. for 90 min. The mixture was cooled to room temperature and poured into water (20 mL). The precipitate was collected and washed with aqueous ammonium hydroxide (0.1 N, 10 mL) to give the title compound (0.87 g, 3.85 mmol, 85%) as a tan powder. LCMS: 97%, Rt 0.969, ESMS m/z 226 (M+H)⁺.

### Preparation 4-1. 2-(1-Bromopropenyl)-1-methyl-3-vinyl-1H-pyrrole

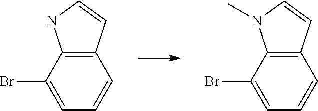

To a suspension of 2-(1-bromopropenyl)-3-vinyl-1H-pyrrole (500 mg, 2.55 mmol) and potassium hydroxide (571 mg, 10.20 mmol) in dimethylsulfoxide (5.1 mL) was added methyl iodide (724 mg, 318 μL, 5.10 mmol) dropwise at room temperature, and the mixture was stirred for 30 min. The mixture was poured into saturated ammonium chloride (25 mL) and extracted with diethyl ether (3×25 mL). The combined organic layers were dried over sodium sulfate and evaporated to give the title compound (518 mg, 2.47 mmol, 97%) as an off-white powder. LCMS: 100%, Rt 1.763, ESMS m/z no ionization.

### Preparation 5-1. 8-Bromo-1,4-dihydro-2H-isoquinolin-3-one

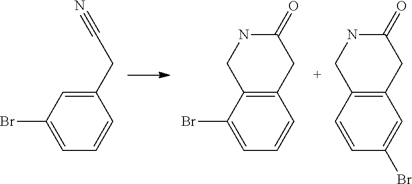

A mixture of paraformaldehyde (337 mg, 11.2 mmol), 3-bromophenylacetonitrile (2 g, 10.2 mmol), and polyphosphoric acid (5 mL) was stirred at 180° C. for 15 min under air. The hot mixture was poured into ice water (50 mL) and 10% aqueous potassium carbonate (30 mL) was added to achieve pH 7. The aqueous layer was extracted with dichloromethane (3×100 mL). The combined organic layers were dried over sodium sulfate and evaporated to afford a mixture of 6-bromo-1,4-dihydro-2H-isoquinolin-3-one and 8-bromo-1,4-dihydro-2H-isoquinolin-3-one (2:1 ratio). The crude product was purified by column chromatography eluting with ethyl acetate:chloroform (1:1→2:1) to give the title compound (565 mg, 2.50 mmol, 24%) as a yellow powder. LCMS: 82%, Rt 1.154, ESMS m/z 226 (M+H)⁺.

### Preparation 6-1. 5-Chloro-2H-phthalazin-1-one

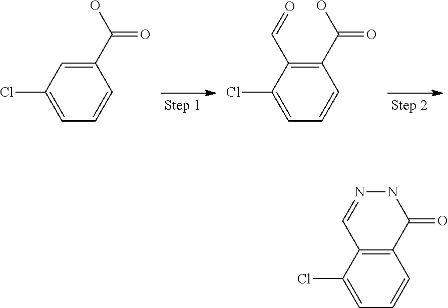 [SMILES: O=C(O)c1cccc(Cl)c1 ; O=Cc1c(Cl)cccc1C(=O)O ; O=c1[nH]ncc2c(Cl)cccc12]

Step 1. To a stirred solution of n-butyllithium (1.6 M in hexane, 8.78 mL, 14.05 mmol) under argon at −20° C. was added 2,2,6,6-tetramethylpiperidine (2.37 mL, 14.05 mmol) in anhydrous tetrahydrofuran (15 mL). The mixture was cooled to −50° C. and a solution of 3-chlorobenzoic acid (1.0 g, 6.39 mmol) in anhydrous tetrahydrofuran (10 mL) was added dropwise. The mixture was stirred for 3 h. The mixture was then treated with N,N-dimethylformamide (1.97 mL, 25.5 mmol) and allowed to warm to room temperature. The mixture was stirred for 18 h. The reaction was quenched with water (5 mL) and the mixture was evaporated. The residue was diluted with hydrochloric acid (2 M, 25 mL) and extracted with diethyl ether (2×25 mL). The combined organic layers were dried over sodium sulfate and evaporated to give 3-chloro-2-formylbenzoic acid (790 mg, 4.28 mmol, 67%) as a yellow powder. LCMS: 72%, Rt 0.988, ESMS m/z 185 (M+H)⁺.

Step 2. To a solution of 3-chloro-2-formylbenzoic acid (Preparation 4a-1, 1.35 g, 7.31 mmol) in water (13.5 mL) was added hydrazine hydrate (1.78 mL, 36.65 mmol), and the mixture was stirred at 95° C. for 4 h. The resulting precipitate was collected, washed with water (5 mL) and dried in air to give the title compound (545 mg, 3.01 mmol, 41%) as a white powder. LCMS: 100%, Rt 1.079, ESMS m/z 181 (M+H)⁺.

### Preparation 7-1. 4-Bromo-3H-benzoxazol-2-one

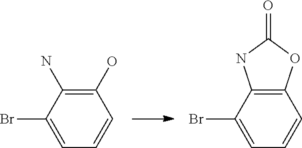 [SMILES: Nc1c(O)cccc1Br ; O=c1[nH]c2c(Br)cccc2o1]

To a solution of 2-amino-3-bromophenol (700 mg, 3.72 mmol) in tetrahydrofuran (60 mL) was added 1,1′-carbonyldiimidazole (1.21 g, 7.44 mmol) and triethylamine (1.04 mL, 7.44 mmol) and the mixture stirred at 60° C. for 2.5 h. The reaction mixture was evaporated and diluted with dichloromethane (60 mL). The organic layer was washed with 1 M hydrochloric acid (2×30 mL) and water (30 mL). The organic layer was dried over sodium sulfate and evaporated to give the title compound (800 mg, 3.74 mmol, ca. 100%) as a light brown powder. LCMS: 100%, Rt 1.191, ESMS m/z 214 (M+H)⁺.

Preparations 7-2—3 listed in the table below were prepared in a similar manner.

| | | | | | Anal. | |
|---|---|---|---|---|---|---|
| Prep. | Structure | MW | Ion | Rt | Method | Yield |
| 7-2 | 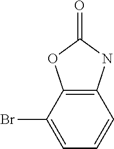 [SMILES: O=c1[nH]c2cccc(Br)c2o1] | 214 | 212 | 1.243 | A | 79 |
| 7-3 | 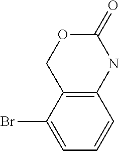 [SMILES: O=C1Nc2cccc(Br)c2CO1] | 228 | 228 | 1.210 | A | 72 |

### Preparation 8-1. 5-Bromo-2H-isoquinolin-1-one

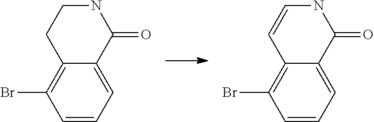 [SMILES: O=C1NCCc2c(Br)cccc21 ; O=c1[nH]ccc2c(Br)cccc12]

A mixture of 5-bromo-3,4-dihydro-2H-isoquinolin-1-one (4.3 g, 18.9 mmol) and 2,3-dicyano-5,6-dichloro-1,4-benzoquinone (8.6 g, 37.9 mmol) in 1,4-dioxane (76 mL) was stirred at 100° C. for 24 h. The reaction mixture was evaporated and the residue was taken up in ethyl acetate (500 mL) and washed with 10% aqueous sodium hydroxide (2×500 mL). The layers were separated and the aqueous layer was extracted with ethyl acetate (4×300 mL). The combined organic layers were dried over sodium sulfate, evaporated and purified by flash chromatography eluting with dichloromethane:methanol (99:1→96:4) to give the title compound (1.49 g, 6.65 mmol, 35%) as a yellow solid. LCMS: 94%, Rt 1.243, ESMS m/z 224 (M+H)⁺.

Preparation 8-2 listed in the table was prepared in a similar manner.

| | | | | | Anal. | |
|---|---|---|---|---|---|---|
| Prep. | Structure | MW | Ion | Rt | Method | Yield |
| 8-2 | 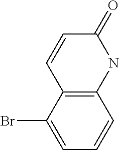 [SMILES: O=c1ccc2c(Br)cccc2[nH]1] | 224 | 226 | 1.290 | A | 39 |

### Preparation 9-1. 4-Bromo-3-(4-methoxybenzyl)-3H-benzoxazol-2-one

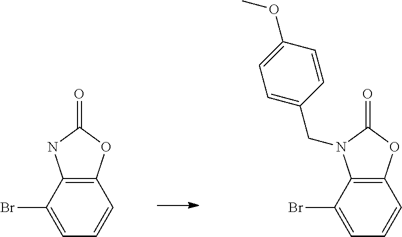 [SMILES: O=c1[nH]c2c(Br)cccc2o1 ; COc1ccc(Cn2c(=O)oc3cccc(Br)c32)cc1]

To a suspension of 4-bromo-3H-benzoxazol-2-one (Preparation 7-1, 664 mg, 3.10 mmol) and cesium carbonate (1.38 g, 4.22 mmol) in acetonitrile (15 mL) was added 4-methoxybenzyl chloride (430 μL, 3.17 mmol) and the mixture stirred at room temperature for 18 h. The mixture was filtered and the solids washed with dichloromethane (3×10 mL). The combined filtrate was evaporated to give the title compound (782 mg, 2.34 mmol, 75%) as an off-white powder. LCMS: 100%, Rt 1.790, ESMS m/z 334 (M+H)⁺.

Preparations 9-2—9-6 listed in the table below were prepared in a similar manner.

| | | | | | Anal. | |
|---|---|---|---|---|---|---|
| Prep. | Structure | MW | Ion | Rt | Method | Yield |
| 9-2 | 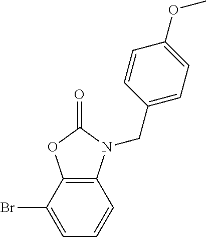 [SMILES: COc1ccc(Cn2c(=O)oc3c(Br)cccc32)cc1] | 334 | 334 | 1.210 | A | 78 |
| 9-3 | 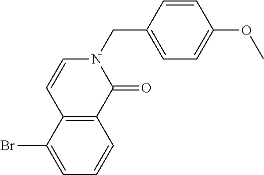 [SMILES: COc1ccc(Cn2ccc3c(Br)cccc3c2=O)cc1] | 344 | 344 | 1.811 | A | 65 |
| 9-4 | 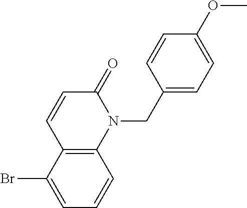 [SMILES: COc1ccc(Cn2c(=O)ccc3c(Br)cccc32)cc1] | 344 | 346 | 1.729 | A | 44 |
| 9-5 | 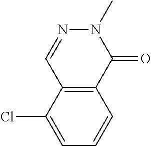 | 195 | 195 | 1.279 | A | 97 |
| 9-6 | 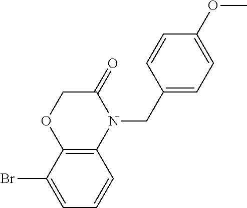 [SMILES: COc1ccc(CN2C(=O)COc3c(Br)cccc32)cc1] | 348 | 348 | 1.748 | A | 89 |

### Preparation 10-1. 8-Bromo-4-(2,4-dimethoxybenzyl)-4H-benzo[1,4]oxazin-3-one

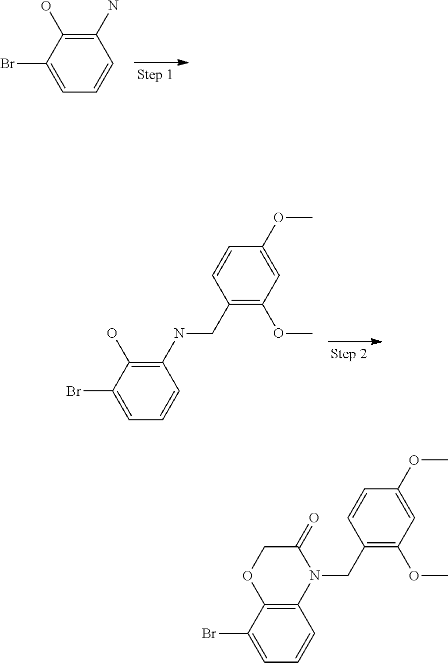 [SMILES: Nc1cccc(Br)c1O ; COc1ccc(CNc2cccc(Br)c2O)c(OC)c1 ; COc1ccc(CN2C(=O)COc3c(Br)cccc32)c(OC)c1]

Step 1. To a solution of 2,4-dimethoxybenzaldehyde (6.64 g, 40.0 mmol) and 2-bromo-6-aminophenol (7.52 g, 40.0 mmol) in 1,2-dichloroethane (120 mL) was added sodium triacetoxyborohydride (10.0 g, 47.1 mmol) in several portions. Acetic acid (300 μL) was added and the mixture stirred at room temperature for 16 h. The mixture was diluted with water (200 mL) and extracted with 1,2-dichloroethane (2×80 mL). The combined organic layers were washed with 5% aqueous sodium bicarbonate (1×40 mL). The organic layer was dried over sodium sulfate and evaporated to give 2-bromo-6-(2,4-dimethoxybenzylamino)-phenol (9.4 g, 27.8 mmol, 70%) as a tan solid. LCMS: 97%.

Step 2. To a solution of 2-bromo-6-(2,4-dimethoxybenzylamino)-phenol (6.76 g, 20.0 mmol) in methyl ethyl ketone (140 mL) cooled with an ice bath was added aqueous potassium carbonate (3.75 M, 16.0 mL, 60.0 mmol) in several portions. The mixture was stirred for 10 min, at which point chloroacetyl chloride (L92 mL, 24.0 mmol) was added dropwise. The reaction mixture was stirred at room temperature for 2 h and then heated at 80° C. for 16 h. The layers were separated and the organic layer was washed with water (80 mL), dried over sodium sulfate and evaporated to give the title compound (6.3 g, 16.7 mmol, 83%) as a light orange solid. LCMS: 97%, Rt 1.816, ESMS m/z 378 (M+H)⁺.

### EXAMPLE 1

### Compound 1-1. 6-Propyl-1,2-dihydro-6H-cyclopenta[c]carbazol-3-one

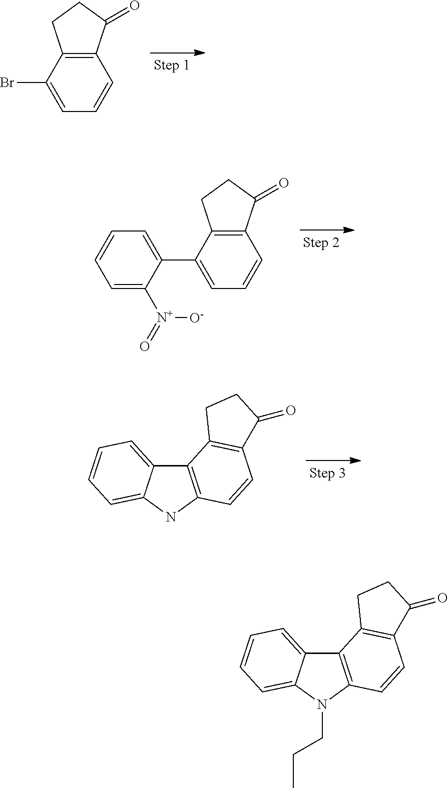 [SMILES: O=C1CCc2c(Br)cccc21 ; O=C1CCc2c1cccc2-c1ccccc1[N+](=O)[O-] ; O=C1CCc2c1ccc1[nH]c3ccccc3c21 ; CCCn1c2ccccc2c2c3c(ccc21)C(=O)CC3]

Step 1. Compound 1a-1. 4-(2-Nitrophenyl)-indan-1-one. A biphasic mixture of 4-bromo-1-indanone (2.0 g, 9.48 mmol), 2-nitrophenylboronic acid (3.16 g, 9.48 mmol), dichloro[1,1′-bis(diphenylphosphino)ferrocene]palladium(II) (348 mg, 0.47 mmol) and potassium carbonate (2.62 g, 9.48 mmol) in 1,4-dioxane:water (4:1, 18 mL) was heated at 120° C. for 30 min under microwave irradiation. The mixture was evaporated and the residue was diluted with water (50 mL) and extracted with dichloromethane (3×50 mL). The combined organic layers were dried over sodium sulfate and evaporated. The residue was purified by column chromatography eluting with hexane:ethyl acetate (100:0→60:40) to give the title compound (1.97 g, 7.78 mmol, 82%) as a yellow crystalline solid. LCMS: 88%, Rt 1.491, ESMS m/z 254 (M+H)⁺.

Compounds 1a-2—1a-8 shown in the table below were prepared in a similar manner using the appropriate aryl bromide.

| | | | | | Anal. | |
|---|---|---|---|---|---|---|
| Ex. | Structure | MW | Ion | Rt | Method | Yield |
| 1a- 2 | 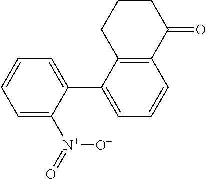 [SMILES: O=C1CCCc2c1cccc2-c1ccccc1[N+](=O)[O-]] | 267 | 268 | 1.686 | A | 87 |
| 1a- 3 | 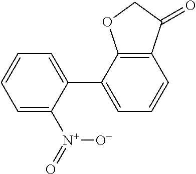 [SMILES: O=C1COc2c1cccc2-c1ccccc1[N+](=O)[O-]] | 255 | 256 | 1.423 | A | 21 |
| 1a- 4 | 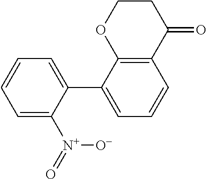 [SMILES: O=C1CCOc2c1cccc2-c1ccccc1[N+](=O)[O-]] | 269 | 270 | 1.515 | A | 88 |
| 1a- 5 | 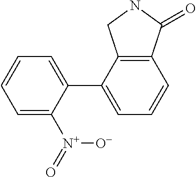 [SMILES: O=C1NCc2c1cccc2-c1ccccc1[N+](=O)[O-]] | 254 | 255 | 1.240 | A | 71 |
| 1a- 6 | 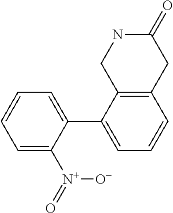 [SMILES: O=C1Cc2cccc(-c3ccccc3[N+](=O)[O-])c2CN1] | 268 | 269 | 1.302 | A | 79 |
| 1a- 7 | 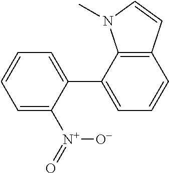 [SMILES: Cn1ccc2cccc(-c3ccccc3[N+](=O)[O-])c21] | 252 | 253 | 1.775 | A | 64 |
| 1a- 8 | 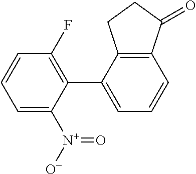 [SMILES: O=C1CCc2c1cccc2-c1c(F)cccc1[N+](=O)[O-]] | 271 | 272 | 1.548 | A | 59 |

Step 2. Compound 1b-1. 1,2-Dihydro-6H-cyclopenta[c]carbazol-3-one. A mixture of 4-(2-nitrophenyl)-indan-1-one (Compound 1a-1, 1.94 g, 7.76 mmol) and triphenylphosphine (5.02 g, 19.17 mmol) in chlorobenzene (38 mL) was heated at 200° C. for 90 min under microwave irradiation. The mixture was evaporated and the residue purified by column chromatography eluting with hexane:acetone (60:40). The product was crystallized from acetone (8 mL) to give the title compound (1.07 g, 4.85 mmol, 63%) as an off-white powder. LCMS: 96%, Rt 1.424, ESMS m/z 222 (M+H)⁺.

Compounds 1b-2—1b-8 shown in the table below were prepared in a similar manner from the appropriate nitroaryl intermediate.

| | | | | | Anal. | |
|---|---|---|---|---|---|---|
| Ex. | Structure | MW | Ion | Rt | Method | Yield |
| 1b-2 | 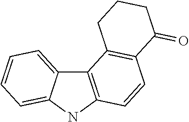 [SMILES: O=C1CCCc2c1ccc1[nH]c3ccccc3c21] | 235 | 236 | 1.588 | A | 44 |
| 1b-3 | 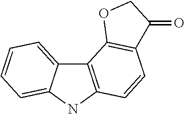 [SMILES: O=C1COc2c1ccc1[nH]c3ccccc3c21] | 223 | 224 | 1.388 | A | 84 |
| 1b-4 | 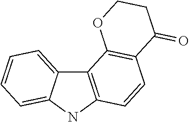 [SMILES: O=C1CCOc2c1ccc1[nH]c3ccccc3c21] | 237 | 238 | 1.475 | A | 34 |
| 1b-5 | 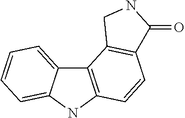 [SMILES: O=C1NCc2c1ccc1[nH]c3ccccc3c21] | 222 | 223 | 1.185 | A | 32 |
| 1b-6 | 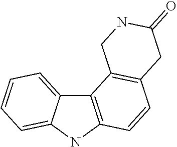 [SMILES: O=C1Cc2ccc3[nH]c4ccccc4c3c2CN1] | 236 | 237 | 1.216 | A | 33 |
| 1b-7 | 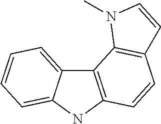 [SMILES: Cn1ccc2ccc3[nH]c4ccccc4c3c21] | 220 | 221 | 1.689 | A | 83 |
| 1b-8 | 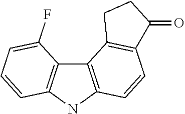 [SMILES: O=C1CCc2c1ccc1[nH]c3cccc(F)c3c21] | 239 | 240 | 1.616 | A | 57 |

Step 3. Compound 1-1. 6-Propyl-1,2-dihydro-6H-cyclopenta[c]carbazol-3-one. To a suspension of 1,2-dihydro-6H-cyclopenta[c]carbazol-3-one (Compound 1b-1, 500 mg, 2.26 mmol) and cesium carbonate (1.47 g, 4.52 mmol) in acetonitrile (23 mL) was added 1-bromopropane (557 mg, 411 μL, 4.52 mmol) dropwise at room temperature. The mixture was stirred at 80° C. for 2 h. The mixture was evaporated, diluted with water (20 mL) and extracted with dichloromethane (2×25 mL). The combined organic layers were dried over sodium sulfate and evaporated. The crude product was crystallized from ethyl acetate (4 mL) to give the title compound (612 mg, 2.32 mmol, ca. 100%) as a brown powder. LCMS: 99%, Rt 1.752, ESMS m/z 264 (M+H)⁺; ¹H NMR (500 MHz, DMSO) δ ppm 8.15 (d, J=7.7 Hz, 1H), 7.76 (d, J=8.2 Hz, 1H), 7.67-7.73 (m, 2H), 7.55 (t, J=7.3 Hz, 1H), 7.34 (t, J=7.4 Hz, 1H), 4.47 (t, J=7.0 Hz, 2H), 3.52-3.62 (m, 2H), 2.72-2.80 (m, 2H), 1.82 (sext, J=7.4 Hz, 2H), 0.87 (t, J=7.4 Hz, 3H).

Compounds 1-2—1-16 shown in the table below were prepared in a similar manner using the appropriate carbazole and alkylating agent.

| | | | | | Anal. | |
|---|---|---|---|---|---|---|
| Ex. | Structure | MW | Ion | Rt | Method | Yield |
| 1-2 | 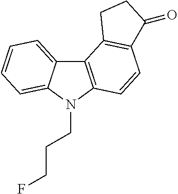 [SMILES: O=C1CCc2c1ccc1c2c2ccccc2n1CCCF] | 281 | 282 | 1.638 | A | ~100 |
| 1-3 | 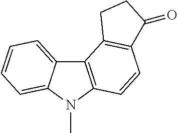 [SMILES: Cn1c2ccccc2c2c3c(ccc21)C(=O)CC3] | 235 | 236 | 1.596 | A | 53 |
| 1-4 | 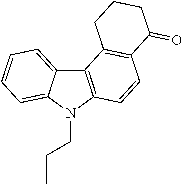 [SMILES: CCCn1c2ccccc2c2c3c(ccc21)C(=O)CCC3] | 277 | 278 | 1.882 | A | 9 |
| 1-5 | 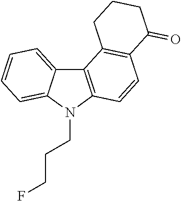 [SMILES: O=C1CCCc2c1ccc1c2c2ccccc2n1CCCF] | 295 | 296 | 1.794 | A | 67 |
| 1-6 | 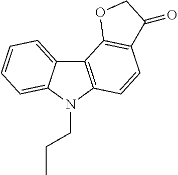 [SMILES: CCCn1c2ccccc2c2c3c(ccc21)C(=O)CO3] | 265 | 266 | 1.731 | A | 40 |
| 1-7 | 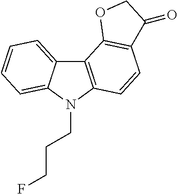 [SMILES: O=C1COc2c1ccc1c2c2ccccc2n1CCCF] | 283 | 284 | 1.668 | A | 54 |
| 1-8 | 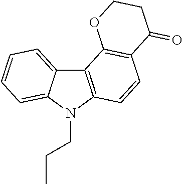 [SMILES: CCCn1c2ccccc2c2c3c(ccc21)C(=O)CCO3] | 279 | 280 | 1.850 | A | 29 |
| 1-9 | 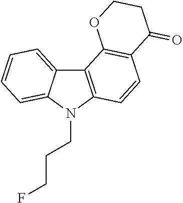 [SMILES: O=C1CCOc2c1ccc1c2c2ccccc2n1CCCF] | 297 | 298 | 1.749 | A | 34 |
| 1-10 | 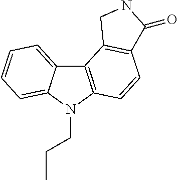 [SMILES: CCCn1c2ccccc2c2c3c(ccc21)C(=O)NC3] | 264 | 265 | 1.519 | A | 68 |
| 1-11 | 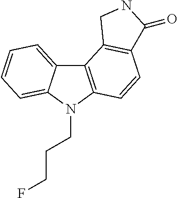 [SMILES: O=C1NCc2c1ccc1c2c2ccccc2n1CCCF] | 282 | 283 | 1.416 | A | 20 |
| 1-12 | 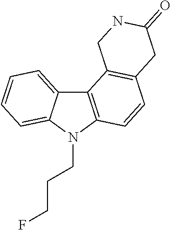 [SMILES: O=C1Cc2ccc3c(c2CN1)c1ccccc1n3CCCF] | 296 | 297 | 2.98 | B | 15 |
| 1-13 | 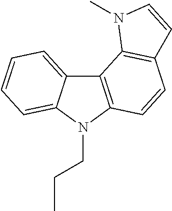 [SMILES: CCCn1c2ccccc2c2c3c(ccc21)ccn3C] | 262 | 263 | 1.976 | A | 100 |
| 1-14 | 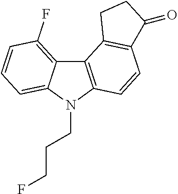 [SMILES: O=C1CCc2c1ccc1c2c2c(F)cccc2n1CCCF] | 299 | 300 | 1.745 | A | 81 |
| 1-15 | 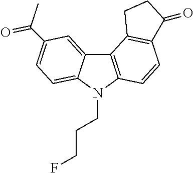 [SMILES: CC(=O)c1ccc2c(c1)c1c3c(ccc1n2CCCF)C(=O)CC3] | 323 | 324 | 1.556 | A | 45 |
| 1-16 | 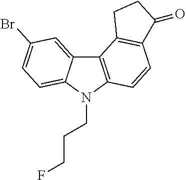 [SMILES: O=C1CCc2c1ccc1c2c2cc(Br)ccc2n1CCCF] | 360 | 362 | 1.843 | A | 39 |

### EXAMPLE 2

### Compound 2-1. 7-Propyl-2,3-dihydro-1H,7H-pyrido[3,4-c]carbazol-4-one

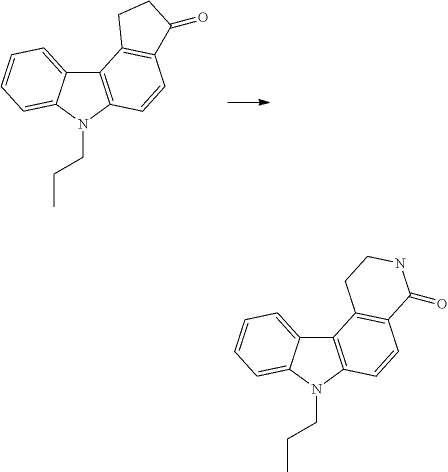 [SMILES: CCCn1c2ccccc2c2c3c(ccc21)C(=O)CC3 ; CCCn1c2ccccc2c2c3c(ccc21)C(=O)NCC3]

To a mixture of 6-propyl-1,2-dihydro-6H-cyclopenta[c]carbazol-3-one (Compound 1-1, 210 mg, 0.80 mmol) and methanesulfonic acid (850 μL, 13.1 mmol) in dichloromethane (8 mL) was added sodium azide (103 mg, 1.60 mmol) and the mixture stirred at 0° C. for 1 h. The reaction mixture was poured into 20% aqueous sodium hydroxide (30 mL) and extracted with dichloromethane (3×30 mL). The combined organic layers were dried over sodium sulfate and evaporated to give the title compound (207 mg, 0.74 mmol, 93%) as a tan solid. LCMS: 84%, Rt 1.611, ESMS m/z 279 (M+H)⁺. The product was used in the next step without further purification. ¹H NMR (500 MHz, CDCl₃) δ ppm 8.27 (d, J=8.8 Hz , 1H), 8.17 (d, J=7.8 Hz , 1H), 7.45-7.55 (m, 2H), 7.40 (d, J=8.8 Hz , 1H), 7.30 (t, J=7.3 Hz , 1H), 5.97 (br. s., 1H), 4.32 (t, J=7.1 Hz , 2H), 3.73-3.80 (m, 2H), 3.66 (t, J=6.6 Hz , 2H), 1.94 (sext, J=7.4 Hz, 2H), 1.00 (t, J=7.4 Hz, 3H).

Compounds 2-2—2-5 shown in the table below were prepared in a similar manner using the appropriate ketone.

| | | | | | Anal. | |
|---|---|---|---|---|---|---|
| | | | | | Meth- | |
| Ex. | Structure | MW | Ion | Rt | od | Yield |
| 2-2 | 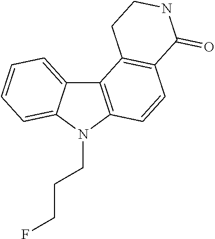 [SMILES: O=C1NCCc2c1ccc1c2c2ccccc2n1CCCF] | 296 | 297 | 1.483 | A | 63 |
| 2-3 | 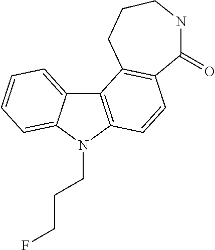 [SMILES: O=C1NCCCc2c1ccc1c2c2ccccc2n1CCCF] | 310 | 311 | 1.532 | A | 20 |
| 2-4 | 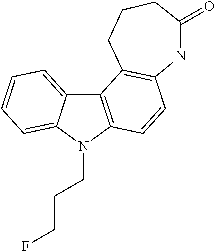 [SMILES: O=C1CCCc2c(ccc3c2c2ccccc2n3CCCF)N1] | 310 | 311 | 1.601 | A | 13 |
| 2-5 | 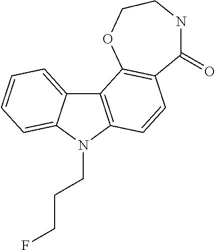 [SMILES: O=C1NCCOc2c1ccc1c2c2ccccc2n1CCCF] | 312 | 313 | 1.538 | A | 78 |

### EXAMPLE 3

### Compound 3-1. 7-Propyl-3H,7H-pyrido[3,4-c]carbazol-4-one

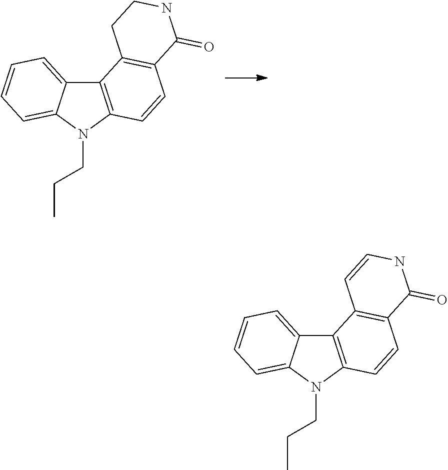 [SMILES: CCCn1c2ccccc2c2c3c(ccc21)C(=O)NCC3 ; CCCn1c2ccccc2c2c3cc[nH]c(=O)c3ccc21]

A mixture of 7-propyl-2,3-dihydro-1H,7H-pyrido[3,4-c]carbazol-4-one (Compound 2-1, 170 mg, 0.61 mmol) and 2,3-dicyano-5,6-dichloro-1,4-benzoquinone (277 mg, 1.22 mmol) in 1,4-dioxane (2.5 mL) was stirred at 100° C. for 14 h. The reaction mixture was evaporated and the residue was taken up in dichloromethane (10 mL) and washed with 10% aqueous sodium hydroxide (2×10 mL). The organic layer was dried over sodium sulfate, evaporated and purified by column chromatography eluting with dichloromethane:methanol (99:1→95:5) to give the title compound (41 mg, 0.15 mmol, 24%) as an off-white solid. LCMS: 98%, Rt 1.598, ESMS m/z 277 (M+H)⁺; ¹H NMR (500 MHz, DMSO-d₆) δ ppm 11.26 (br. s, 1H), 8.46 (d, J=7.8 Hz, 1H), 8.30 (d, J=8.8 Hz, 1H), 7.80 (d, J=8.8 Hz, 1H), 7.77 (d, J=8.3 Hz, 1H), 7.48-7.55 (m, 1H), 7.36-7.44 (m, 2H), 7.31-7.36 (m, 1H), 4.49 (t, J=7.1 Hz, 2H), 1.83 (sext, J=7.3 Hz, 2H), 0.89 (t, J=7.3 Hz, 3H).

Compounds 3-2—3-3 shown in the table below were prepared in a similar manner using the appropriate dihydro compound.

| | | | | | Anal. | |
|---|---|---|---|---|---|---|
| | | | | | Meth- | |
| Ex. | Structure | MW | Ion | Rt | od | Yield |
| 3-2 | 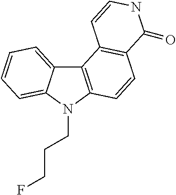 [SMILES: O=c1[nH]ccc2c1ccc1c2c2ccccc2n1CCCF] | 294 | 295 | 1.487 | A | 17 |
| 3-3 | 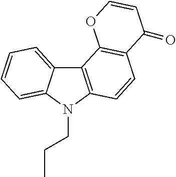 [SMILES: CCCn1c2ccccc2c2c3occc(=O)c3ccc21] | 277 | 278 | 1.750 | A | 40 |

### EXAMPLE 4

### Compound 4-1. 2-Methyl-6-propyl-1,2-dihydro-6H-pyrrolo[3,4-c]carbazol-3-one

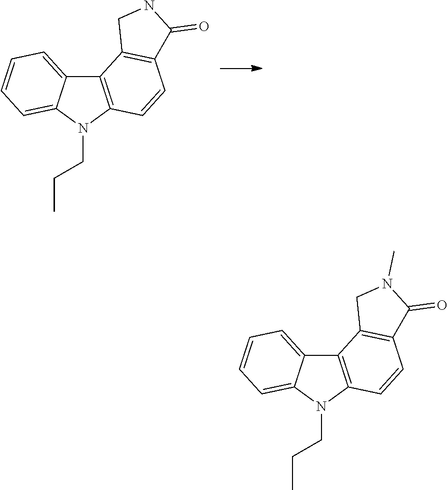 [SMILES: CCCn1c2ccccc2c2c3c(ccc21)C(=O)NC3 ; CCCn1c2ccccc2c2c3c(ccc21)C(=O)N(C)C3]

To a solution of 6-propyl-1,2-dihydro-6H-pyrrolo[3,4-c]carbazol-3-one (Compound 1-10, 45 mg, 0.17 mmol) in acetonitrile (1 mL) was added cesium carbonate (123 mg, 0.38 mmol) and methyl iodide (27 μL, 0.34 mmol). The mixture was stirred at room temperature for 2 h. The mixture was evaporated and the residue was taken up in dichloromethane (4 mL) and water (2 mL). The layers were separated and the aqueous layer was extracted with dichloromethane (2×2 mL). The combined organic layers were dried over sodium sulfate, evaporated, and the residue was purified by column chromatography eluting with hexane:ethyl acetate (80:20→0:100) to give the title compound (12 mg, 43.2 μmol, 24%) as a pale yellow powder. LCMS: 92%, Rt 1.635, ESMS m/z 279 (M+H)⁺, ¹H NMR (300 MHz, CDCl₃) δ ppm 7.87-7.96 (m, 2H), 7.42-7.58 (m, 3H), 7.28-7.36 (m, 1H), 4.80 (s, 2H), 4.34 (t, J=7.3 Hz, 2H), 3.30 (s, 3H), 1.95 (sext, J=7.3 Hz, 2H), 0.99 (t, J=7.3 Hz, 3H).

Compounds 4-2—4-4 shown in the table below were prepared in a similar manner using the appropriate fused carbazole.

| | | | | | Anal. | |
|---|---|---|---|---|---|---|
| Ex. | Structure | MW | Ion | Rt | Method | Yield |
| 4-2 | 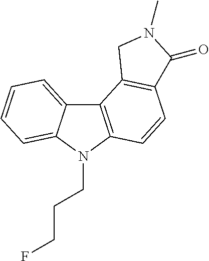 [SMILES: CN1Cc2c(ccc3c2c2ccccc2n3CCCF)C1=O] | 296 | 297 | 1.548 | A | 11 |
| 4-3 | 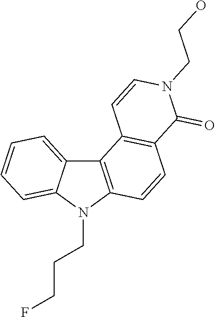 [SMILES: O=c1c2ccc3c(c2ccn1CCO)c1ccccc1n3CCCF] | 338 | 339 | 1.467 | A | 31 |
| 4-4 | 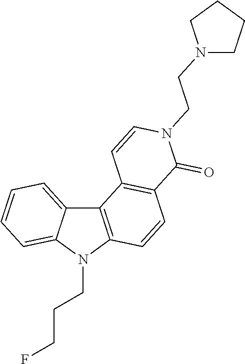 [SMILES: O=c1c2ccc3c(c2ccn1CCN1CCCC1)c1ccccc1n3CCCF] | 391 | 392 | 1.294 | A | 27 |

### EXAMPLE 5

### Compound 5-1. 7-Propyl-3H-pyrimido[5,4-c]carbazol-4(7H)-one

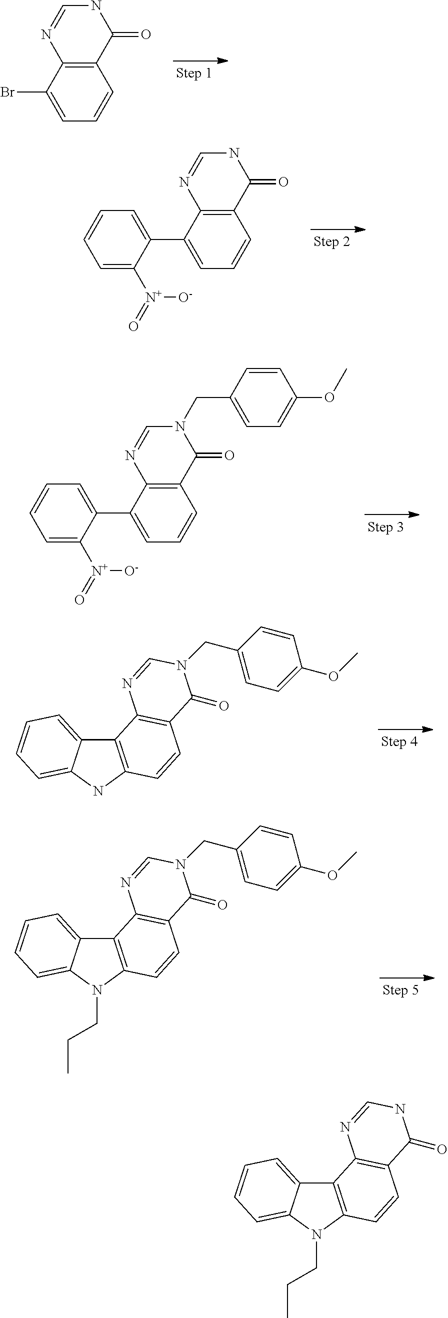 [SMILES: O=c1[nH]cnc2c(Br)cccc12 ; O=c1[nH]cnc2c(-c3ccccc3[N+](=O)[O-])cccc12 ; COc1ccc(Cn2cnc3c(-c4ccccc4[N+](=O)[O-])cccc3c2=O)cc1 ; COc1ccc(Cn2cnc3c(ccc4[nH]c5ccccc5c43)c2=O)cc1 ; CCCn1c2ccccc2c2c3ncn(Cc4ccc(OC)cc4)c(=O)c3ccc21 ; CCCn1c2ccccc2c2c3nc[nH]c(=O)c3ccc21]

Step 1. Compound 5a-1. 8-(2-Nitrophenyl)quinazolin-4(3H)-one. A biphasic mixture of 8-bromo-3H-quinazolin-4-one (Preparation 1-1, 0.42 g, 1.87 mmol), 2-nitrophenylboronic acid (0.70 g, 4.20 mmol), aqueous potassium carbonate (2 M, 1.87 mL, 3.73 mmol) and dichloro[1,1′-bis(diphenylphosphino)ferrocene]palladium(II) (205 mg, 0.28 mmol) in 1,4-dioxane (15 mL) under argon was heated at 120° C. for 30 min under microwave irradiation. The mixture was evaporated and the residue was diluted with water (30 mL) and extracted with dichloromethane (3×30 mL). The combined organic layers were dried over sodium sulfate and evaporated. The residue was purified by column chromatography eluting with ethyl acetate:hexane (60:40) to give the title compound (0.23 g, 0.86 mmol, 45%) as a tan solid. LCMS: 97%, Rt 1.233, ESMS m/z 267 (M+H)⁻;

Compound 5a-2 shown in the table below was prepared in a similar manner using the appropriate aryl bromide.

| | | | | | Anal. | |
|---|---|---|---|---|---|---|
| | | | | | Meth- | |
| Ex. | Structure | MW | Ion | Rt | od | Yield |
| 5a- 2 | 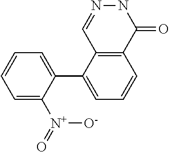 [SMILES: O=c1[nH]ncc2c(-c3ccccc3[N+](=O)[O-])cccc12] | 267 | 268 | 1.300 | A | 26 |

Step 2. Compound 5b-1. 3-(4-Methoxybenzyl)-8-(2-nitrophenyl)quinazolin-4(3H)-one. To a suspension of 8-(2-nitrophenyl)quinazolin-4(3H)-one (Compound 5a-1, 200 mg, 0.75 mmol) and cesium carbonate (488 mg, 1.50 mmol) in acetonitrile (7.5 mL) was added 4-methoxybenzyl chloride (152 μL, 1.13 mmol) and the mixture stirred at room temperature for 18 h. The mixture was evaporated, diluted with water (10 mL) and extracted with ethyl acetate (3×10 mL). The combined organic layers were dried over sodium sulfate and evaporated to give the title compound (290 mg, 0.75 mmol, ca. 100%) as a tan solid. LCMS: 83%, Rt 1.709, ESMS m/z 388 (M+H)⁺. The crude product was used in the next step without further purification.

Compound 5b-2 shown in the table below was prepared in a similar manner.

| | | | | | Anal. | |
|---|---|---|---|---|---|---|
| Ex. | Structure | MW | Ion | Rt | Method | Yield |
| 5b-2 | 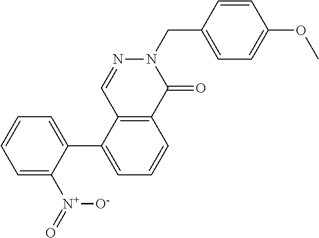 [SMILES: COc1ccc(Cn2ncc3c(-c4ccccc4[N+](=O)[O-])cccc3c2=O)cc1] | 387 | 388 | 1.780 | A | 57 |

Step 3. Compound 5c-1. 3-(4-Methoxybenzyl)-3H-pyrimido[5,4-c]carbazol-4(7H)-one. A mixture of 3-(4-methoxybenzyl)-8-(2-nitrophenyl)quinazolin-4(3H)-one (Compound 5b-1, 290 mg, 0.75 mmol) and 1,2-bis(diphenylphosphino)ethane (492 mg, 1.24 mmol) was stirred at 200° C. for 2 h. The mixture was purified by column chromatography eluting with hexane:ethyl acetate (95:5→50:50) to give the title compound (100 mg, 0.28 mmol, 34%) as a brown powder. LCMS: 88%, Rt 1.712, ESMS m/z 356 (M+H)⁺.

Compound 5c-2 shown in the table below was prepared in a similar manner.

| | | | | | Anal. | |
|---|---|---|---|---|---|---|
| Ex. | Structure | MW | Ion | Rt | Method | Yield |
| 5c-2 | 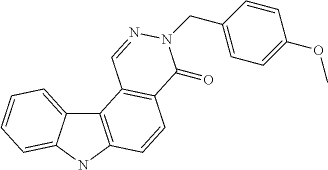 [SMILES: COc1ccc(Cn2ncc3c(ccc4[nH]c5ccccc5c43)c2=O)cc1] | 355 | 356 | 1.741 | A | crude |

Step 4. Compound 5d-1. 3-(4-Methoxybenzyl)-7-propyl-3H-pyrimido[5,4-c]carbazol-4(7H)-one. To a suspension of 3-(4-methoxybenzyl)-3H-pyrimido[5,4-c]carbazol-4(7H)-one (Compound 5c-1, 100 mg, 0.28 mmol) and cesium carbonate (184 mg, 0.56 mmol) in acetonitrile (2.8 mL) was added 1-bromopropane (30 μL, 0.34 mmol) at room temperature. The mixture was heated to 60° C. for 2 h. The mixture was evaporated, diluted with water (5 mL) and extracted with ethyl acetate (3×5 mL). The combined organic layers were dried over sodium sulfate and evaporated to give the title compound (94 mg, 0.24 mmol, 84%) as a brown powder. LCMS: 92%, Rt 2.055, ESMS m/z 398 (M+H)⁺.

Compounds 5d-2—5-3 shown in the table below were prepared in a similar manner from the appropriate carbazole intermediate and alkylating agent.

| | | | | | Anal. | |
|---|---|---|---|---|---|---|
| Ex. | Structure | MW | Ion | Rt | Method | Yield |
| 5d-2 | 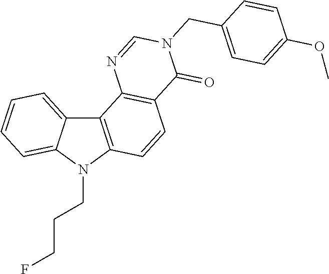 [SMILES: COc1ccc(Cn2cnc3c(ccc4c3c3ccccc3n4CCCF)c2=O)cc1] | 415 | 416 | 1.917 | A | 87 |
| 5d-3 | 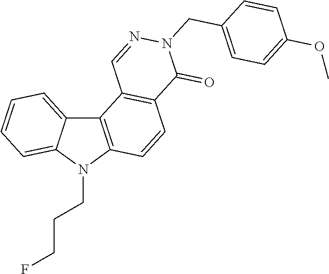 [SMILES: COc1ccc(Cn2ncc3c(ccc4c3c3ccccc3n4CCCF)c2=O)cc1] | 415 | 416 | 1.923 | A | 14 |

Step 5. Compound 5-1. 7-Propyl-3H-pyrimido[5,4-c]carbazol-4(7H)-one.

A mixture of 3-(4-methoxybenzyl)-7-propyl-3H-pyrimido[5,4-c]carbazol-4(7H)-one (Compound 5d-1, 47 mg, 0.12 mmol) in trifluoroacetic acid (1 mL) was heated at 70° C. for 20 h. The mixture was then heated to 100° C. for 4 h. The mixture was evaporated, diluted with dichloromethane (5 mL) and washed with 10% aqueous sodium bicarbonate (5 mL) and water (5 mL). The organic layer was dried over sodium sulfate and evaporated. The crude product was purified by column chromatography eluting with hexane:ethyl acetate (60:40) to give the title compound (10 mg, 0.03 mmol, 29%) as an off-white powder. LCMS: 100%, Rt 1.649, ESMS m/z 278 (M+H)⁺; ¹H NMR (500 MHz, DMSO-d₆) δ ppm 12.22 (br. s, 1H), 8.75 (d, J=7.8 Hz, 1H), 8.31 (s, 1H), 8.16 (d, J=8.8 Hz, 1H), 7.82 (d, J=8.8 Hz, 1H), 7.74 (d, J=8.3 Hz, 1H), 7.46-7.54 (m, 1H), 7.32 (t, J=7.8 Hz, 1H), 4.49 (t, J=7.1 Hz, 2H), 1.84 (sext, J=7.3 Hz, 2H), 0.89 (t, J=7.3 Hz, 3H).

Compounds 5-2—5-3 shown in the table below were prepared in a similar manner using the appropriate carbazole.

| | | | | | Anal. | |
|---|---|---|---|---|---|---|
| Ex. | Structure | MW | Ion | Rt | Method | Yield |
| 5-2 | 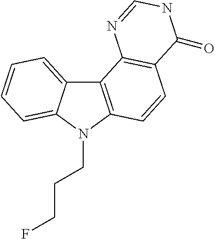 [SMILES: O=c1[nH]cnc2c1ccc1c2c2ccccc2n1CCCF] | 295 | 296 | 1.479 | A | 8 |
| 5-3 | 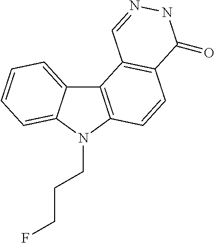 [SMILES: O=c1[nH]ncc2c1ccc1c2c2ccccc2n1CCCF] | 295 | 296 | 1.444 | A | 16 |

### EXAMPLE 6

### Compound 6-1 1-Methyl-6-propyl-3,6-dihydro-1H-pyrrolo[3,2-c]carbazol-2-one

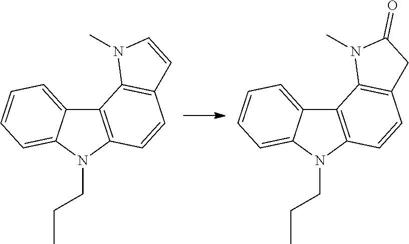 [SMILES: CCCn1c2ccccc2c2c3c(ccc21)ccn3C ; CCCn1c2ccccc2c2c3c(ccc21)CC(=O)N3C]

To a solution of 1-methyl-6-propyl-1,6-dihydropyrrolo[3,2-c]carbazole (Compound 1-19, 100 mg, 0.38 mmol) in acetic acid (1.9 mL) was added magnesium monoperoxyphthalate hexahydrate (190 mg, 0.38 mmol) and the reaction mixture was stirred at room temperature for 1 h. The reaction mixture was evaporated and the residue was diluted with water (5 mL). The aqueous mixture was made basic to pH 10 by addition of solid sodium carbonate, then extracted with dichloromethane (3×5 mL). The combined organic layers were dried over sodium sulfate and evaporated. The residue was purified by column chromatography eluting with hexane:ethyl acetate (4:1) to give the title compound (10 mg, 0.04 mmol, 9%) as a gray powder. LCMS: 100%, Rt 1.787, ESMS m/z 279 (M+H)⁺; ¹H NMR (300 MHz, DMSO-d₆) δ ppm 8.41 (d, J=8.3 Hz, 1H), 7.63 (d, J=8.1 Hz, 1H), 7.45 (t, J=7.6 Hz, 1H), 7.35 (d, J=8.3 Hz, 1H), 7.26 (d, J=8.3 Hz, 1H), 7.17 (t, J=7.6 Hz, 1H), 4.35 (t, J=7.1 Hz, 2H), 3.81 (s, 3H), 3.67 (s, 2H), 1.77 (sext, J=7.3 Hz, 2H), 0.88 (t, J=7.3 Hz, 3H).

### EXAMPLE 7

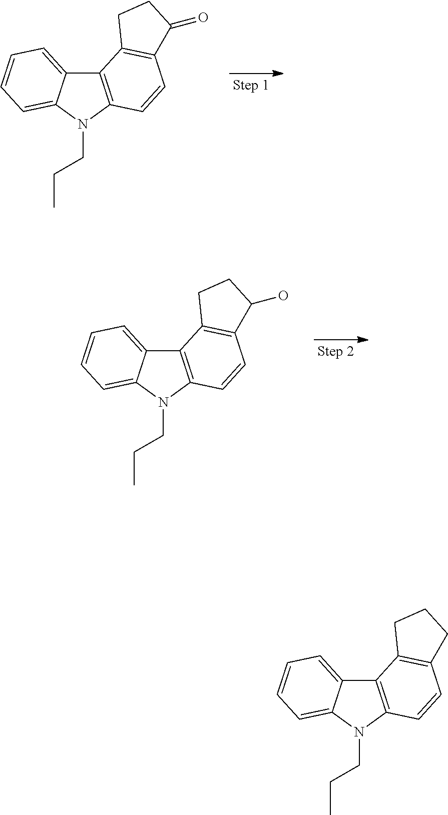 [SMILES: CCCn1c2ccccc2c2c3c(ccc21)C(=O)CC3 ; CCCn1c2ccccc2c2c3c(ccc21)C(O)CC3 ; CCCn1c2ccccc2c2c3c(ccc21)CCC3]

Step 1. Compound 7-1. 6-Propyl-1,2,3,6-tetrahydro-cyclopenta[c]carbazol-3-ol. To a solution of 6-propyl-1,2-dihydro-6H-cyclopenta[c]carbazol-3-one (Compound 1-1, 100 mg, 0.38 mmol) in methanol (0.6 mL) was added sodium borohydride (58 mg, 1.52 mmol) and the reaction mixture was stirred at room temperature for 1 h. Water (2.5 mL) was added and the reaction mixture was extracted with ethyl acetate (3×3 mL). The combined organic layers were dried over sodium sulfate and evaporated. The residue was purified by column chromatography eluting with hexane:ethyl acetate (60:40) to give the title compound (31 mg, 0.12 mmol, 31%) as a pale yellow powder. LCMS: 95%, Rt 1.703, ESMS m/z 266 (M+H)⁺. ¹H NMR (500 MHz, CDCl₃) δ ppm 8.08 (d, J=7.8 Hz, 1H), 7.55 (d, J=8.3 Hz, 1H), 7.43-7.50 (m, 2H), 7.34 (d, J=8.3 Hz, 1H), 7.26 (t, J=7.5 Hz, 2H), 5.43-5.49 (m, 1H), 4.31 (t, J=7.2 Hz, 2H), 3.58-3.65 (m, 1H), 3.31-3.40 (m, 1H), 2.67-2.77 (m, 1H),2.18-2.26 (m, 1H), 1.88-1.98 (m, 2H), 1.70 (d, J=7.2 Hz, 1H), 0.99 (t, J=7.4 Hz, 3H).

Step 2. Compound 7-2. 6-Propyl-1,2,3,6-tetrahydro-cyclopenta[c]carbazole. To a solution of 6-propyl-1,2,3,6-tetrahydro-cyclopenta[c]carbazol-3-ol (Compound 7-1, 136 mg, 0.51 mmol) in dichloromethane (3.5 mL) was added trifluoroacetic acid (3.5 mL) and triethylsilane (130 mg, 179 μL, 1.12 mmol) and the mixture was stirred at room temperature for 20 h. The reaction mixture was evaporated and the residue was purified by column chromatography eluting with hexane:ethyl acetate (98:2) to give the title compound (25 mg, 0.10 mmol, 20%) as a white gum. LCMS: 100%, Rt 2.172, ESMS m/z 250 (M+H)⁺; ¹H NMR (500 MHz, CDCl₃) δ ppm 8.08 (d, J=7.8 Hz , 4H), 7.39-7.47 (m, 2H), 7.37 (d, J=8.3 Hz, 4H), 7.18-7.26 (m, 2H), 4.29 (t, J=7.1 Hz, 2H), 3.45 (t, J=7.3 Hz, 2H), 3.10 (t, J =7.3 Hz , 2H), 2.26-2.37 (m, 2H), 1.92 (sext, J=7.4 Hz, 2H), 0.99 (t, J=7.4 Hz, 3H).

### EXAMPLE 8

### Compound 8-1. 6-Propyl-1H,6H-3-oxa-1,6-diazacyclopenta[c]fluoren-2-one

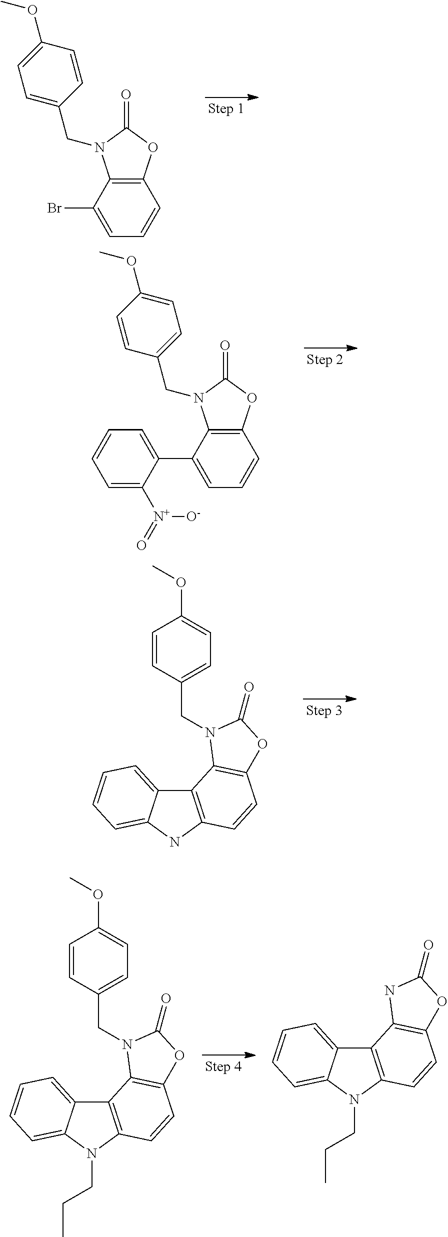

Step 1. Compound 8a-1. 3-(4-Methoxybenzyl)-4-(2-nitrophenyl)-3H-benzooxazol-2-one. A biphasic mixture of 4-bromo-3-(4-methoxybenzyl)-3H-benzooxazol-2-one (Preparation 9-1, 860 mg, 2.57 mmol), 2-nitrophenylboronic acid (575 mg, 3.09 mmol), aqueous potassium carbonate (2 M, 2.57 mL, 5.14 mmol) and dichloro[1,1′-bis(diphenylphosphino)ferrocene]palladium(II) (94 mg, 0.13 mmol) in 1,4-dioxane (16 mL) was heated at 120° C. for 60 min by microwave irradiation under argon. The mixture was evaporated and the residue was purified by column chromatography eluting with hexane:ethyl acetate (100:065:35) to give the title compound (540 mg, 1.44 mmol, 56%) as an off-white solid. LCMS: 92%, Rt 1.766, ESMS m/z 377 (M+H)⁺.

Compounds 8a-2—8a-4 shown in the table below were prepared in a similar manner.

| | | | | | Anal. | |
|---|---|---|---|---|---|---|
| Ex. | Structure | MW | Ion | Rt | Method | Yield |
| 8a-2 | 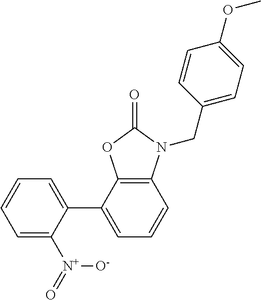 [SMILES: COc1ccc(Cn2c(=O)oc3c(-c4ccccc4[N+](=O)[O-])cccc32)cc1] | 376 | 394 | 1.799 | A | 56 |
| 8a-3 | 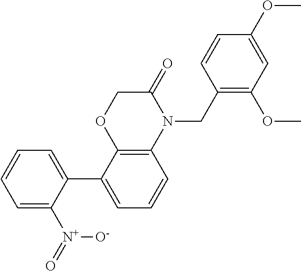 [SMILES: COc1ccc(CN2C(=O)COc3c(-c4ccccc4[N+](=O)[O-])cccc32)c(OC)c1] | 420 | 421 | 1.846 | A | 65 |
| 8a-4 | 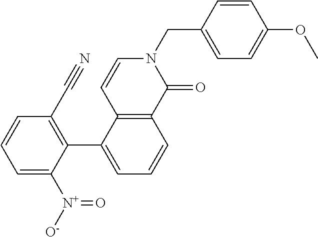 [SMILES: COc1ccc(Cn2ccc3c(-c4c(C#N)cccc4[N+](=O)[O-])cccc3c2=O)cc1] | 411 | 412 | 1.651 | A | 59 |

Step 2. Compound 8b-1. 1-(4-Methoxybenzyl)-1H,6H-3-oxa-1,6-diazacyclopenta[c]fluoren-2-one. A mixture of 3-(4-methoxybenzyl)-4-(2-nitrophenyl)-3H-benzoxazol-2-one (Compound 8a-1, 450 mg, 1.20 mmol) and triphenylphosphine (784 mg, 3.00 mmol) was stirred at 200° C. for 2 h. The mixture was purified by column chromatography eluting with hexane:ethyl acetate (90:10→60:40) to give the title compound contaminated with triphenylphosphine oxide (455 mg, 1.32 mmol, ca. 100%) as a brown powder. LCMS: 31%, Rt 1.697, ESMS m/z 345 (M+H)⁺.

Compounds 8b-2—8b-4 shown in the table below were prepared in a similar manner.

| | | | | | Anal. | |
|---|---|---|---|---|---|---|
| Ex. | Structure | MW | Ion | Rt | Method | Yield |
| 8b-2 | 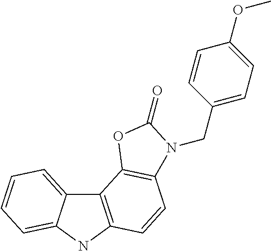 [SMILES: COc1ccc(Cn2c(=O)oc3c4c(ccc32)[nH]c2ccccc24)cc1] | 344 | 345 | 1.765 | A | 37 |
| 8b-3 | 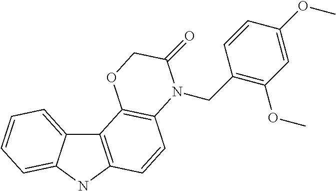 [SMILES: COc1ccc(CN2C(=O)COc3c2ccc2[nH]c4ccccc4c32)c(OC)c1] | 388 | 389 | 1.782 | A | 47 |
| 8b-4 | 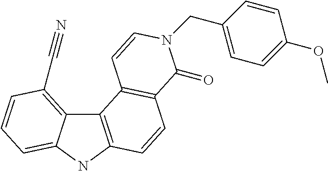 [SMILES: COc1ccc(Cn2ccc3c(ccc4[nH]c5cccc(C#N)c5c43)c2=O)cc1] | 379 | 380 | 1.696 | A | 34 |

Step 3. Compound 8c-1 1-(4-Methoxybenzyl)-6-(3-fluoropropyl)-1H,6H-3-oxa-1,6-diazacyclopenta[c]fluoren-2-one. To a suspension of 1-(4-methoxybenzyl)-1H,6H-3-oxa-1,6-diazacyclopenta[c]fluoren-2-one (Compound 8b-1, 205 mg, 0.59 mmol) and cesium carbonate (391 mg, 1.2 mmol) in N,N-dimethylacetamide (3 mL) was added 1-iodo-3-fluoropropane (91 μL, 0.89 mmol) at room temperature. The mixture was stirred at 60° C. for 15 h. The mixture was evaporated, diluted with water (5 mL) and extracted with dichloromethane (2×5 mL). The combined organic layers were dried over sodium sulfate and evaporated. The crude product was purified by column chromatography eluting with hexane:ethyl acetate (90:10→60:40) to give the title compound (95 mg, 0.28 mmol, 40%) as an off-white powder. LCMS: 97%, Rt 1.899, ESMS m/z 404 (M+H)⁺.

Compounds 8c-2—4 shown in the table below were prepared in a similar manner using the appropriate carbazole.

| | | | | | Anal. | |
|---|---|---|---|---|---|---|
| Ex. | Structure | MW | Ion | Rt | Method | Yield |
| 8c-2 | 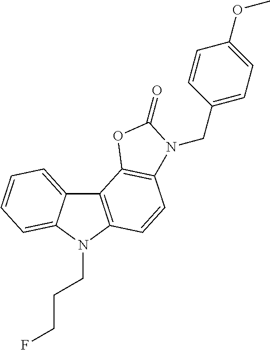 [SMILES: COc1ccc(Cn2c(=O)oc3c4c5ccccc5n(CCCF)c4ccc32)cc1] | 404 | 405 | 1.968 | A | 94 |
| 8c-3 | 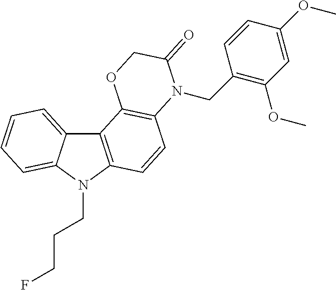 [SMILES: COc1ccc(CN2C(=O)COc3c2ccc2c3c3ccccc3n2CCCF)c(OC)c1] | 448 | 449 | 1.968 | A | 84 |
| 8c-4 | 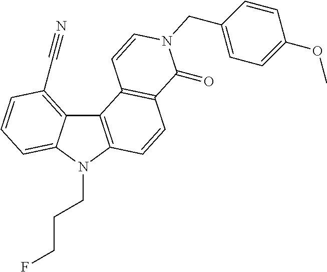 [SMILES: COc1ccc(Cn2ccc3c(ccc4c3c3c(C#N)cccc3n4CCCF)c2=O)cc1] | 439 | 440 | 1.873 | A | 96 |

Step 4. Compound 8-1. 6-(3-Fluoropropyl)-1H,6H-3-oxa-1,6-diazacyclopenta[c]fluoren-2-one. A solution of 1-(4-methoxybenzyl)-6-propyl-1H,6H-3-oxa-1,6-diazacyclopenta[c]fluoren-2-one (Compound 8c-1, 95 mg, 0.24 mmol) in trifluoroacetic acid (1 mL) was stirred at 80° C. for 2 h. The mixture was evaporated and the residue taken up in dichloromethane (3 mL), washed with 10% aqueous sodium bicarbonate (3 mL) and water (3 mL), dried over sodium sulfate and evaporated. The crude product was purified by preparative HPLC to give the title compound (3 mg, 0.01 mmol, 5%) as an off-white powder. LCMS: 95%, Rt 1.558, ESMS m/z 285 (M+H)⁺; ¹H NMR (500 MHz, CDCl₃) δ ppm 8.24 (d, J=7.8 Hz, 1H), 7.53 (t, J=7.6 Hz, 1H), 7.45-7.48 (m, 1H), 7.39 (d, J=8.8 Hz, 1H), 7.35 (t, J=7.3 Hz, 1H), 7.16 (d, J=8.8 Hz, 1H), 4.50 (t, J=6.6 Hz, 2H), 4.43 (dt, J=47.0, 5.9 Hz , 2H), 2.27 (dquint, J=27.9, 5.9 Hz , 2H).

Compounds 8-2—8-3 shown in the table below below were prepared in a similar manner. Compound 8-4 was isolated as a minor product from the 8-3 reaction mixture.

| | | | | | Anal. | |
|---|---|---|---|---|---|---|
| Ex. | Structure | MW | Ion | Rt | Method | Yield |
| 8-2 | 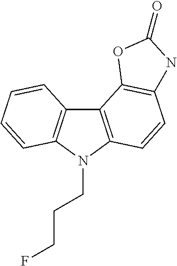 [SMILES: O=c1[nH]c2ccc3c(c4ccccc4n3CCCF)c2o1] | 284 | 285 | 1.564 | A | 5 |
| 8-3 | 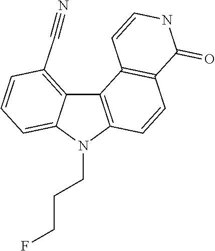 [SMILES: N#Cc1cccc2c1c1c3cc[nH]c(=O)c3ccc1n2CCCF] | 319 | 320 | 1.455 | A | 33 |
| 8-4 | 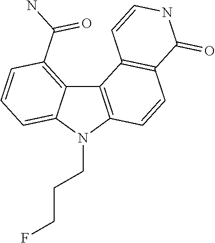 [SMILES: NC(=O)c1cccc2c1c1c3cc[nH]c(=O)c3ccc1n2CCCF] | 337 | 338 | 1.107 | A | 3 |

### EXAMPLE 9

### Compound 9-1. 7-(3-Fluoropropyl)-4,7-dihydro-1-oxa-4,7-diaza-benzo[c]fluoren-3-one

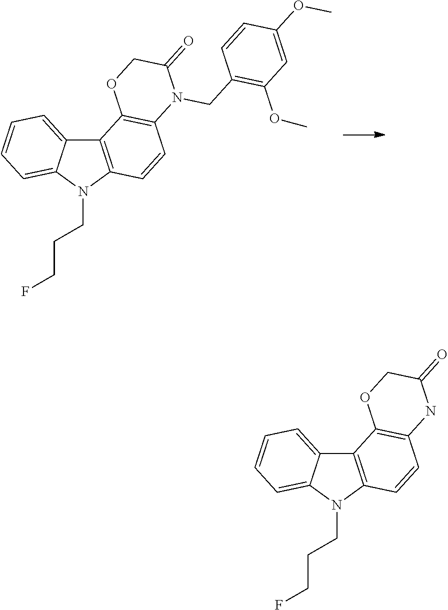 [SMILES: COc1ccc(CN2C(=O)COc3c2ccc2c3c3ccccc3n2CCCF)c(OC)c1 ; O=C1COc2c(ccc3c2c2ccccc2n3CCCF)N1]

A mixture of 4-(2,4-dimethoxybenzyl)-7-(3-fluoropropyl)-4,7-dihydro-1-oxa-4,7-diazabenzo[c]-fluoren-3-one (Compound 8c-3, 448 mg, 1.00 mmol) and triethylsilane (480 μL, 3.00 mmol) in trifluoroacetic acid (3.0 mL) was stirred at 65° C. for 2.5 h. The precipitate was collected by filtration, washed with saturated aqueous sodium carbonate (2×1.5 mL) and dried in air. The crude product was purified by column chromatography eluting with hexane:ethyl acetate (2:1) to give the title compound (116 mg, 0.389 mmol, 38%) as an off-white solid. LCMS: 95%, Rt 1.553, ESMS m/z 299 (M+H)⁺. ¹H NMR (300 MHz, DMSO) δ 10.65 (s, 1H), 8.11 (d, J=7.7 Hz, 1H), 7.55 (d, J=8.2 Hz, 1H), 7.44 (t, J=7.4 Hz, 1H) 7.11-7.26 (m, 2H), 7.04 (d, J=8.5 Hz, 1H), 4.80 (s, 2H), 4.27-4.56 (m, 4H), 2.12 (dquint, J=26.7, 6.1 Hz, 2H).

### EXAMPLE 10

### Compound 10-1. 6-Propyl-1,2-dihydro-6H-6,10-diazacyclopenta[c]fluoren-3-one

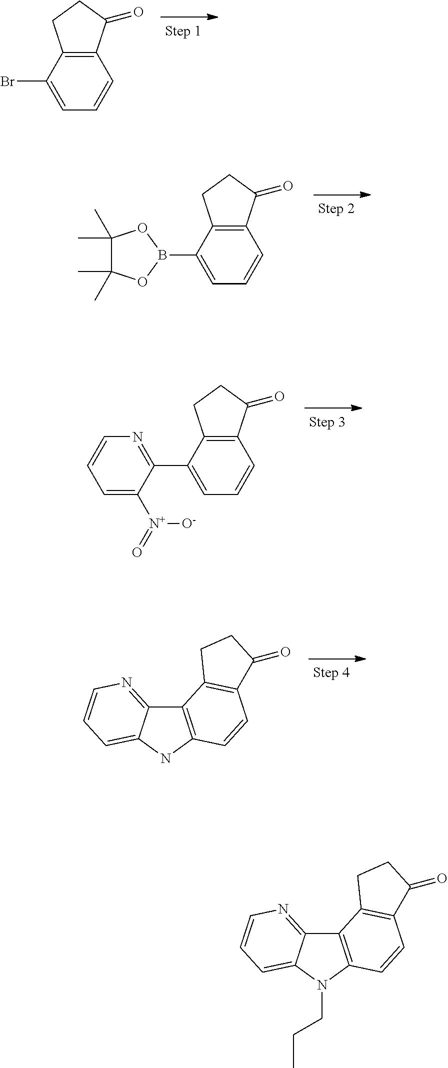 [SMILES: O=C1CCc2c(Br)cccc21 ; CC1(C)OB(c2cccc3c2CCC3=O)OC1(C)C ; O=C1CCc2c1cccc2-c1ncccc1[N+](=O)[O-] ; O=C1CCc2c1ccc1[nH]c3cccnc3c21 ; CCCn1c2cccnc2c2c3c(ccc21)C(=O)CC3]

Step 1. Compound 10a-1. 4-(4,4,5,5-Tetramethyl-1,3,2-dioxaborolan-2-yl)-indan-1-one. To a solution of 4-bromo-1-indanone (1.0 g, 4.74 mmol) in N,N-dimethylacetamide (25 mL) was added bis(pinacolato)diboron (2.41 g, 9.48 mmol), dichloro[1,1′-bis(diphenyl-phosphino)ferrocene] palladium(II) (346 mg, 0.47 mmol) and potassium acetate (1.40 g, 14.22 mmol) and the reaction mixture was stirred at 100° C. for 2 h under nitrogen. The mixture was evaporated and the residue was diluted with water (50 mL) and extracted with dichloromethane (3×50 mL). The combined organic layers were dried over sodium sulfate and evaporated. The residue was purified by column chromatography eluting with hexane:ethyl acetate (100:0→4:1) to give the title compound (1.8 g, 7.00 mmol, ca. 100%) as an off-white powder. LCMS: 97%, Rt 1.811, ESMS m/z 259 (M+H—CH₄)⁺.

Compounds 10a-2—10a-7 listed in the table below were prepared in a similar manner.

| | | | | | Anal. | |
|---|---|---|---|---|---|---|
| Ex. | Structure | MW | Ion | Rt | Method | Yield |
| 10a-2 | 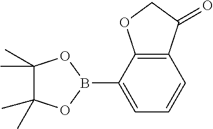 [SMILES: CC1(C)OB(c2cccc3c2OCC3=O)OC1(C)C] | 276 | 261 | 1.601 | A | crude |
| 10a-3 | 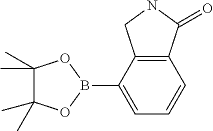 [SMILES: CC1(C)OB(c2cccc3c2CNC3=O)OC1(C)C] | 275 | 260 | 1.485 | A | 36 |
| 10a-4 | 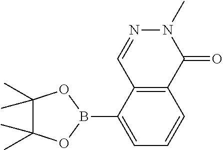 [SMILES: Cn1ncc2c(B3OC(C)(C)C(C)(C)O3)cccc2c1=O] | 286 | 287 | 1.698 | A | 84 |
| 10a-5 | 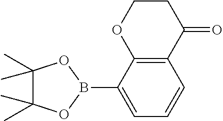 [SMILES: CC1(C)OB(c2cccc3c2OCCC3=O)OC1(C)C] | 274 | 275 | 1.566 | A | 62 |
| 10a-6 | 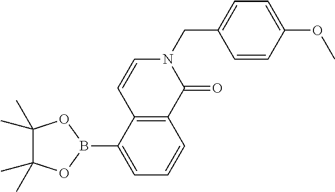 | 391 | 392 | 1.981 | A | ~100 |
| 10a-7 | 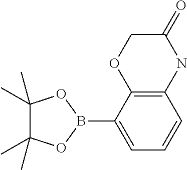 [SMILES: CC1(C)OB(c2cccc3c2OCC(=O)N3)OC1(C)C] | 275 | 276 | 1.367 | A | 77 |

Step 2. Compound 10b-1. 4-(3-Nitropyridin-2-yl)-indan-1-one. A biphasic mixture of 4-(4,4,5,5-tetramethyl-1,3,2-dioxaborolan-2-yl)-indan-1-one (Compound 10a-1, 1.6 g, 6.20 mmol), 2-bromo-3-nitropyridine (1.51 g, 7.45 mmol), dichloro[1,1′-bis(diphenyl-phosphino)ferrocene]palladium(II) (452 mg, 0.62 mmol) and 2 M aqueous potassium carbonate (6.2 mL, 12 4 mmol) in 1,4-dioxane (62 mL) under nitrogen was stirred at 120° C. for 45 min under microwave irradiation in 4 separate portions. The reaction mixtures were combined and evaporated. The residue was diluted with water (60 mL) and extracted with dichloromethane (3×60 mL). The combined organic layers were dried over sodium sulfate and evaporated. The residue was purified by column chromatography eluting with hexane:ethyl acetate (85:15→40:60) to give the title compound (1.1 g, 4.33 mmol, 70%) as a yellow powder. LCMS: 100%, Rt 1.308, ESMS m/z 255 (M+H)⁺.

Compounds 10b-2—7 listed in the table below were prepared in a similar manner.

| | | | | | Anal. | |
|---|---|---|---|---|---|---|
| Ex. | Structure | MW | Ion | Rt | Method | Yield |
| 10b-2 | 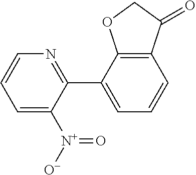 [SMILES: O=C1COc2c1cccc2-c1ncccc1[N+](=O)[O-]] | 256 | 257 | 1.285 | A | 95 |
| 10b-3 | 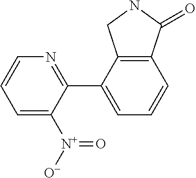 [SMILES: O=C1NCc2c1cccc2-c1ncccc1[N+](=O)[O-]] | 255 | 256 | 1.081 | A | 24 |
| 10b-4 | 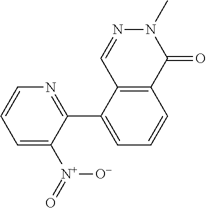 [SMILES: Cn1ncc2c(-c3ncccc3[N+](=O)[O-])cccc2c1=O] | 282 | 283 | 1.189 | A | 76 |
| 10b-5 | 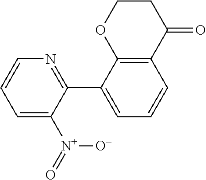 [SMILES: O=C1CCOc2c1cccc2-c1ncccc1[N+](=O)[O-]] | 270 | 271 | 1.309 | A | 56 |
| 10b-6 | 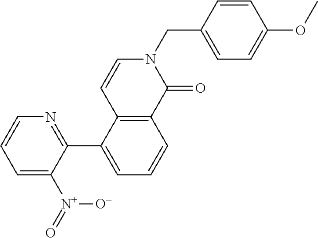 [SMILES: COc1ccc(Cn2ccc3c(-c4ncccc4[N+](=O)[O-])cccc3c2=O)cc1] | 387 | 388 | 1.565 | A | 72 |
| 10b-7 | 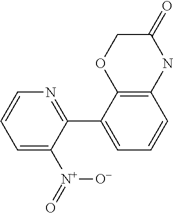 [SMILES: O=C1COc2c(cccc2-c2ncccc2[N+](=O)[O-])N1] | 271 | 272 | 1.130 | A | 60 |

Step 3. Compound 10c-1. 1,2-Dihydro-6H-6,10-diazacyclopenta[c]fluoren-3-one. A mixture of 4-(3-nitropyridin-2-yl)-indan-1-one (Compound 10b-1, 600 mg, 2.36 mmol) and triphenylphosphine (1.55 g, 5.90 mmol) in chlorobenzene (11 mL) under nitrogen was heated at 200° C. for 35 min under microwave irradiation. The mixture was evaporated and the residue was purified by column chromatography eluting with dichloromethane:methanol (100:0→90:10) to give the title compound (411 mg, 1.85 mmol, 78%) as an off-white powder. LCMS: 100%, Rt 0.863, ESMS m/z 223 (M+H)⁺.

Compounds 10c-2—10c-7 listed in the table below were prepared in a similar manner. Compound 10c-7 was prepared in two steps from Compound 10b-7 treating first with 4-methoxybenzyl chloride by the method of Preparation 9.

| | | | | | Anal. | |
|---|---|---|---|---|---|---|
| Ex. | Structure | MW | Ion | Rt | Method | Yield |
| 10c-2 | 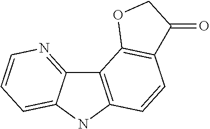 [SMILES: O=C1COc2c1ccc1[nH]c3cccnc3c21] | 224 | 225 | 0.75 | A | 27 |
| 10c-3 | 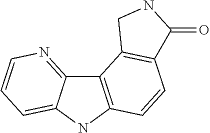 [SMILES: O=C1NCc2c1ccc1[nH]c3cccnc3c21] | 223 | 224 | 0.684 | A | Crude |
| 10c-4 | 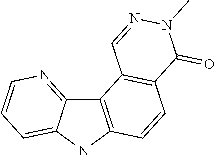 [SMILES: Cn1ncc2c(ccc3[nH]c4cccnc4c32)c1=O] | 250 | 251 | 1.140 | A | 70 |
| 10c-5 | 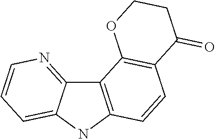 [SMILES: O=C1CCOc2c1ccc1[nH]c3cccnc3c21] | 238 | 239 | 0.770 | A | 35 |
| 10c-6 | 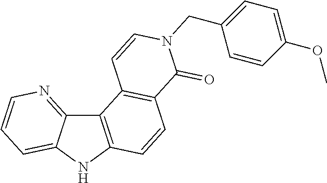 [SMILES: COc1ccc(Cn2ccc3c(ccc4[nH]c5cccnc5c43)c2=O)cc1] | 355 | 356 | 1.416 | A | 65 |
| 10c-7 | 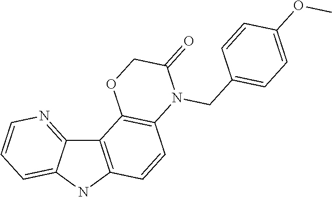 [SMILES: COc1ccc(CN2C(=O)COc3c2ccc2[nH]c4cccnc4c32)cc1] | 359 | 360 | 1.127 | A | 38 |

Step 4. Compound 10d-1. 6-Propyl-1,2-dihydro-6H-6,10-diaza-cyclopenta[c]fluoren-3-one. To a suspension of 1,2-dihydro-6H-6,10-diaza-cyclopenta[c]fluoren-3-one (Compound 10c-1, 211 mg, 0.95 mmol) and cesium carbonate (619 mg, 1.90 mmol) in acetonitrile (9.5 mL) was added 1-bromopropane (175 mg, 129 μL, 1.43 mmol) dropwise at room temperature. The mixture was stirred at 80° C. for 90 min. The mixture was evaporated, diluted with water (10 mL) and extracted with dichloromethane (2×10 mL). The combined organic layers were dried over sodium sulfate and evaporated. The crude product was crystallized from ethyl acetate (3 mL) to give the title compound (211 mg, 0.80 mmol, 84%) as a tan powder. LCMS: 100%, ESMS Rt 1.317, m/z 265 (M+H)⁺; ¹H NMR (500 MHz, DMSO-d₆) δ ppm 8.62 (d, J=4.4 Hz, 1H), 8.19 (d, J=8.3 Hz, 1H), 7.72-7.81 (m, 2H), 7.52 (dd, J=8.3, 4.4 Hz, 1H), 4.49 (t, J=6.8 Hz, 2H), 3.59-3.68 (m, 2H), 2.70-2.79 (m, 2H), 1.82 (sext, 2H), 0.86 (t, 3H)

Compounds 10-2—10-10 listed in the table below were prepared in a similar manner using the appropriate carbazole and alkylating agent.

| | | | | | Anal. | |
|---|---|---|---|---|---|---|
| Ex. | Structure | MW | Ion | Rt | Method | Yield |
| 10-2 | 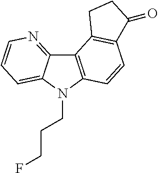 [SMILES: O=C1CCc2c1ccc1c2c2ncccc2n1CCCF] | 282 | 283 | 1.226 | A | 80 |
| 10-3 | 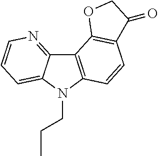 [SMILES: CCCn1c2cccnc2c2c3c(ccc21)C(=O)CO3] | 266 | 267 | 1.178 | A | 17 |
| 10-4 | 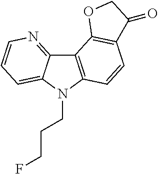 [SMILES: O=C1COc2c1ccc1c2c2ncccc2n1CCCF] | 284 | 285 | 1.092 | A | 9 |
| 10-5 | 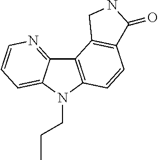 [SMILES: CCCn1c2cccnc2c2c3c(ccc21)C(=O)NC3] | 265 | 266 | 1.186 | A | 8 |
| 10-6 | 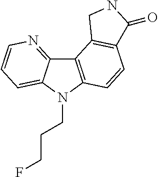 [SMILES: O=C1NCc2c1ccc1c2c2ncccc2n1CCCF] | 283 | 284 | 1.117 | A | 11 |
| 10-7 | 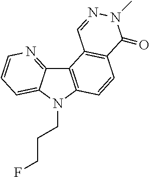 [SMILES: Cn1ncc2c(ccc3c2c2ncccc2n3CCCF)c1=O] | 310 | 311 | 1.435 | A | 24 |
| 10-8 | 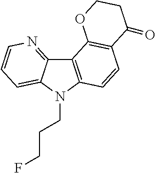 [SMILES: O=C1CCOc2c1ccc1c2c2ncccc2n1CCCF] | 298 | 299 | 1.004 | A | 100 |
| 10-9 | 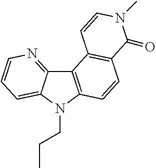 [SMILES: CCCn1c2cccnc2c2c3ccn(C)c(=O)c3ccc21] | 291 | 292 | 1.431 | A | 35 |
| 10-10 | 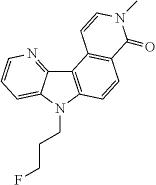 [SMILES: Cn1ccc2c(ccc3c2c2ncccc2n3CCCF)c1=O] | 309 | 310 | 1.339 | A | 13 |

### EXAMPLE 11

### Compound 11-1. 7-Propyl-1,2,3,7-tetrahydro-3,7,11-triazabenzo[c]fluoren-4-one

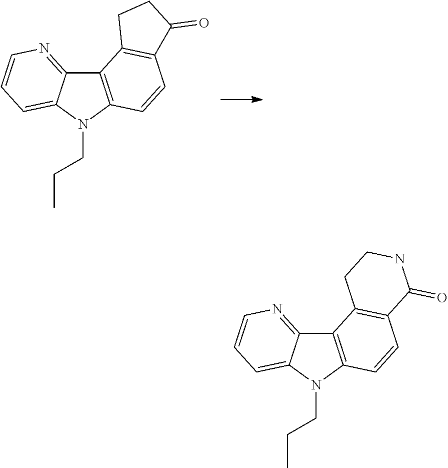 [SMILES: CCCn1c2cccnc2c2c3c(ccc21)C(=O)CC3 ; CCCn1c2cccnc2c2c3c(ccc21)C(=O)NCC3]

To a mixture of 6-propyl-1,2-dihydro-6H-6,10-diaza-cyclopenta[c]fluoren-3-one (Compound 10-1, 200 mg, 0.76 mmol) and methanesulfonic acid (800 μL, 12.4 mmol) in dichloromethane (7 mL) was added sodium azide (99 mg, 1.52 mmol) in several portions at 0° C. The mixture was stirred at 0° C. for 1 h. The mixture was warmed to room temperature and stirred for 20 h. The reaction mixture was poured into 20% aqueous sodium hydroxide (20 mL) and extracted with dichloromethane (3×20 mL). The combined organic layers were washed with water, dried over sodium sulfate and evaporated. The crude product was crystallized from ethyl acetate (2.5 mL) to give the title compound (153 mg, 0.55 mmol, 73%) as an off-white powder. LCMS: 96%, Rt 1.308, ESMS m/z 280 (M+H)⁺; ¹H NMR (300 MHz, DMSO-d₆) δ ppm 8.53 (d, J=4.5 Hz, 1H), 8.13 (d, J=8.3 Hz, 1H), 8.07 (d, J=8.7 Hz, 1H), 7.79 (br. s, 1H), 7.66 (d, J=8.7 Hz, 1H), 7.47 (dd, J=8.3, 4.5 Hz, 1H), 4.43 (t, J=6.9 Hz, 2H), 3.81 (t, J=6.5 Hz, 2H), 3.47-3.60 (m, 2H), 1.81 (sext, J=7.4 Hz, 2H), 0.86 (t, J=7.4 Hz, 3H).

Compounds 11-2—11-3 listed in the table below were prepared in a similar manner.

| | | | | | Anal. | |
|---|---|---|---|---|---|---|
| | | | | | Meth- | |
| Ex. | Structure | MW | Ion | Rt | od | Yield |
| 11- 2 | 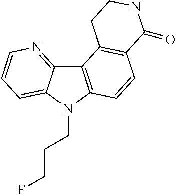 [SMILES: O=C1NCCc2c1ccc1c2c2ncccc2n1CCCF] | 297 | 298 | 1.178 | A | 59 |
| 11- 3 | 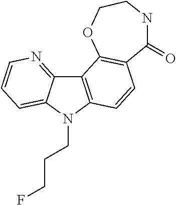 [SMILES: O=C1NCCOc2c1ccc1c2c2ncccc2n1CCCF] | 313 | 314 | 0.878 | A | 19 |

### EXAMPLE 12

### Compound 12-1. 7-Propyl-3,7-dihydro-3,7,11-triazabenzo[c]fluoren-4-one

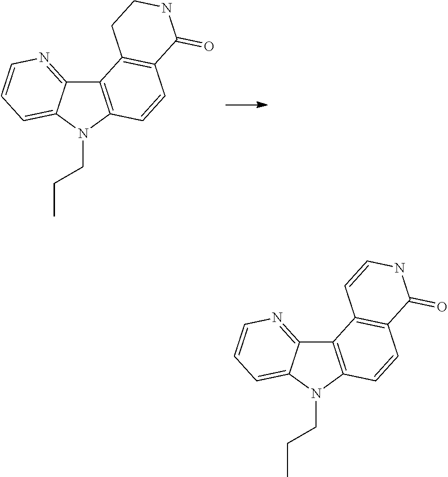 [SMILES: CCCn1c2cccnc2c2c3c(ccc21)C(=O)NCC3 ; CCCn1c2cccnc2c2c3cc[nH]c(=O)c3ccc21]

A mixture of 7-propyl-1,2,3,7-tetrahydro-3,7,11-triazabenzo[c]fluoren-4-one (Compound 11-1, 57 mg, 0.20 mmol) and 2,3-dicyano-5,6-dichloro-1,4-benzoquinone (161 mg, 0.70 mmol) in 1,4-dioxane (0.8 mL) was stirred at 100° C. for 3 d. The reaction mixture was diluted with 1 M aqueous sodium hydroxide (10 mL) and extracted with ethyl acetate (5×10 mL). The combined organic layers were dried over sodium sulfate, evaporated and purified by preparative HPLC to give the title compound (3 mg, 0.01 mmol, 5%) as a pale yellow solid. LCMS: 89%, ESMS Rt 1.320, m/z 278 (M+H)⁺; ¹H NMR (500 MHz, MeOH-d₄, 333K), δ ppm 8.62 (d, J=4.4 Hz, 1H), 8.48 (d, J=9.3 Hz, 1H), 8.32 (d, J=6.8 Hz, 1H), 8.05 (d, J=8.3 Hz, 1H), 7.77 (d, J=9.3 Hz, 1H), 7.47 (dd, J=8.3, 4.4 Hz, 1H), 7.42 (d, J=7.3 Hz, 1H), 4.47-4.52 (m, 2H), 1.96 (sext, J=7.5 Hz, 2H), 0.96 (t, J=7.5 Hz, 3H).

### EXAMPLE 13

### Compound 13-1. 7-(3-Fluoropropyl)-3,7-dihydro-3,7,11-triaza-benzo[c]fluoren-4-one

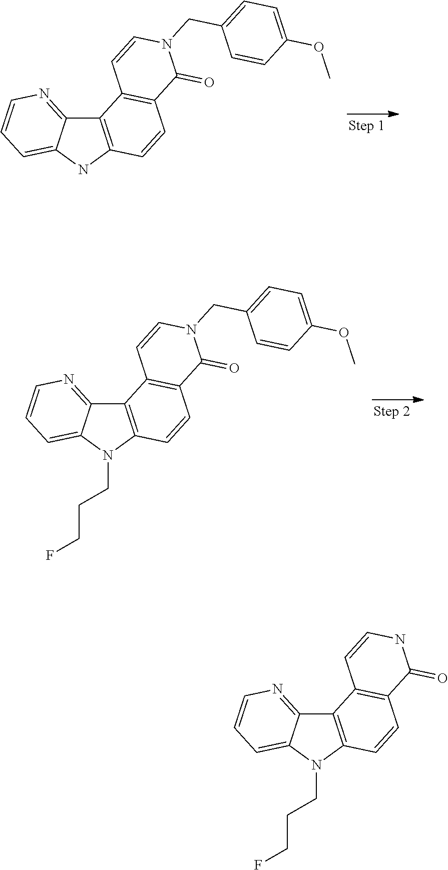 [SMILES: COc1ccc(Cn2ccc3c(ccc4[nH]c5cccnc5c43)c2=O)cc1 ; COc1ccc(Cn2ccc3c(ccc4c3c3ncccc3n4CCCF)c2=O)cc1 ; O=c1[nH]ccc2c1ccc1c2c2ncccc2n1CCCF]

Step 1. Compound 13a-1. 7-(3-Fluoropropyl)-3-(4-methoxybenzyl)-3,7-dihydro-3,7,11-triazabenzo[c]fluoren-4-one.

A mixture of 3-(4-methoxybenzyl)-3,7-dihydro-3,7,11-triazabenzo[c]fluoren-4-one (Compound 10c-6, 200 mg, 0.563 mmol), 1-iodo-3-fluoropropane (86 μL, 0.844 mmol) and cesium carbonate (367 mg, 1.13 mmol) in acetonitrile (3.5 mL) was stirred at 60° C. for 2 h. The reaction mixture was evaporated and the residue was taken up in dichloromethane (20 mL). The organic layer was washed with water (10 mL), dried over sodium sulfate and evaporated to give the title compound (210 mg, 0.505 mmol, 90%) as a gray powder. The product was used in the next step without further purification. LCMS: 91%, Rt 1.715, ESMS m/z 416 (M+H)⁺.

Compound 13a-2 listed in the table below was prepared in a similar manner.

| | | | | | Anal. | |
|---|---|---|---|---|---|---|
| Ex. | Structure | MW | Ion | Rt | Method | Yield |
| 13a-2 | 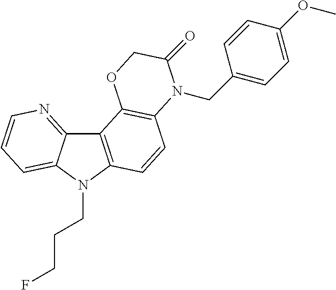 [SMILES: COc1ccc(CN2C(=O)COc3c2ccc2c3c3ncccc3n2CCCF)cc1] | 419 | 420 | 1.304 | A | 100 |

Step 2. Compound 13-1. 7-(3-Fluoropropyl)-3,7-dihydro-3,7,11-triazabenzo[c]fluoren-4-one. A solution of 7-(3-fluoropropyl)-3-(4-methoxybenzyl)-3,7-dihydro-3,7,11-triazabenzo[c]fluoren-4-one (Compound 13a-1, 100 mg, 0.241 mmol) in trifluoroacetic acid (6.0 mL) was irradiated in a microwave reactor at 150° C. for 1 h. The reaction mixture was evaporated and the residue was taken up in dichloromethane (10 mL). The organic layer was washed with 10% aqueous potassium carbonate (2×4 mL), dried over sodium sulfate and evaporated. The residue was purified by column chromatography eluting with dichloromethane:methanol (99:1→96:4). The resulting solid was triturated with diethyl ether to give the title compound (55 mg, 0.186 mmol, 77%) as a light brown powder. LCMS: 98%, Rt 1.230, ESMS m/z 296 (M+H)⁺; ¹H NMR (500 MHz, DMSO) δ 11.34 (bs, 1H), 8.63 (dd, J=4.5, 1.0 Hz, 1H), 8.35 (d, J=8.9 Hz, 1H), 8.16 (dd, J=8.5, 1.0 Hz, 1H), 8.09 (d, J=7.1 Hz, 1H), 7.82 (d, J=8.9 Hz, 1H), 7.48-7.54 (m, 1H), 7.44 (d, J=7.1 Hz, 1H), 4.65 (t, J=6.9 Hz, 2H), 4.44 (dt, J=47.5, 8.9 Hz, 2H), 2.20 (dquint, J=26.5, 6.2 Hz, 2H).

Compound 13b-2 listed in the table below was prepared in a similar manner.

| | | | | | Anal. | |
|---|---|---|---|---|---|---|
| | | | | | Meth- | |
| Ex. | Structure | MW | Ion | Rt | od | Yield |
| 13- 2 | 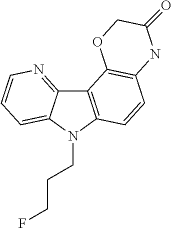 [SMILES: O=C1COc2c(ccc3c2c2ncccc2n3CCCF)N1] | 299 | 300 | 0.892 | A | 66 |

### EXAMPLE 14

### Compound 14-1. 6-(3-Fluoropropyl)-2-methyl-1,2-dihydro-6H-2,6,10-triazacyclopenta[c]fluoren-3-one

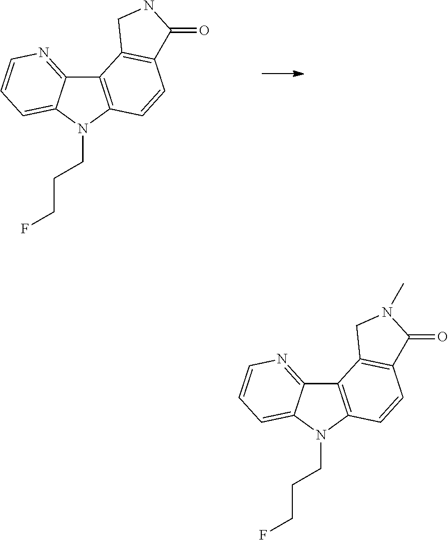 [SMILES: O=C1NCc2c1ccc1c2c2ncccc2n1CCCF ; CN1Cc2c(ccc3c2c2ncccc2n3CCCF)C1=O]

To a mixture of 6-(3-fluoropropyl)-1,2-dihydro-6H-2,6,10-triazacyclopenta[c]fluoren-3-one (Compound 10-6, 88 mg, 0.310 mmol) and cesium carbonate (202 mg, 0.620 mmol) in N,N-dimethylformamide (2 mL) was added iodomethane (23 μL, 0.370 mmol). The reaction mixture stirred at room temperature for 16 h. The mixture was evaporated and the residue purified by column chromatography eluting with dichloromethane:methanol (100:0→95:5). The product was triturated with diethyl ether to afford the title compound (20 mg, 0.067 mmol, 22%) as a pale yellow powder. LCMS: 96%, Rt 1.187, ESMS m/z 298 (M+H)⁺.¹H NMR (500 MHz, DMSO) δ 8.59 (d, J=3.8 Hz, 1H), 8.13 (d, J=7.9 Hz, 1H), 7.72-7.87 (m, 2H), 7.50-7.56 (m, 1H), 4.94 (s, 2H), 4.61 (t, J=6.8 Hz, 2H), 4.43 (dt, J=47.3, 5.6 Hz, 2H), 3.16 (s, 3H), 2.19 (dquint, J=27.0, 6.0 Hz, 2H).

Compound 14-2 listed in the table below was prepared in a similar manner.

| | | | | | Anal. | |
|---|---|---|---|---|---|---|
| Ex. | CHEMISTRY | MW | Ion | Rt | Method | Yield |
| 219 14-2 | 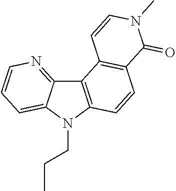 [SMILES: CCCn1c2cccnc2c2c3ccn(C)c(=O)c3ccc21] | 291 | 292 | 1.431 | A | 35 |

### EXAMPLE 15

### Compound 15-1. 7-(3,3-Difluoropropyl)-3,7-dihydro-3,7,11-triaza-benzo[c]fluoren-4-one

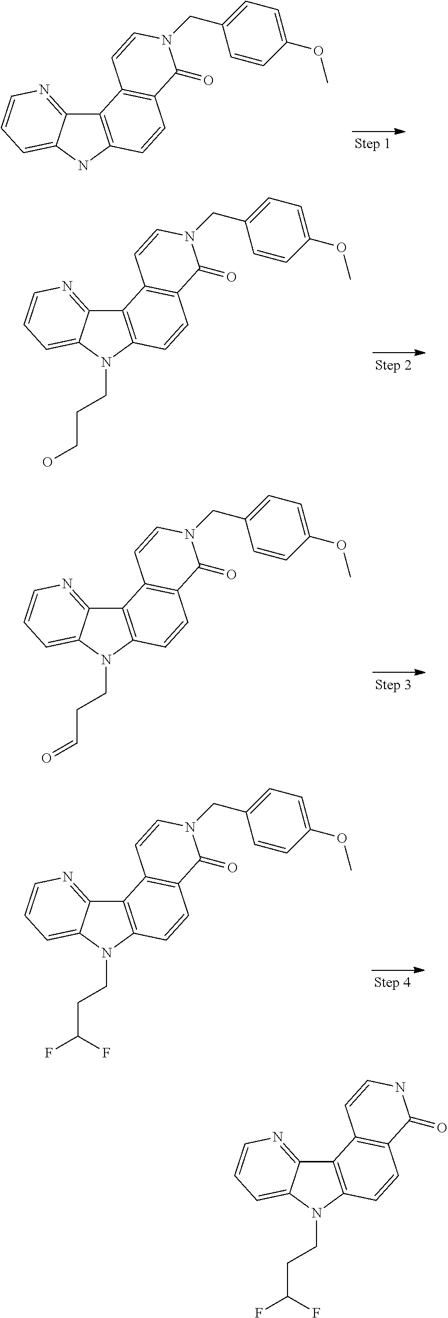 [SMILES: COc1ccc(Cn2ccc3c(ccc4[nH]c5cccnc5c43)c2=O)cc1 ; COc1ccc(Cn2ccc3c(ccc4c3c3ncccc3n4CCCO)c2=O)cc1 ; COc1ccc(Cn2ccc3c(ccc4c3c3ncccc3n4CCC=O)c2=O)cc1 ; COc1ccc(Cn2ccc3c(ccc4c3c3ncccc3n4CCC(F)F)c2=O)cc1 ; O=c1[nH]ccc2c1ccc1c2c2ncccc2n1CCC(F)F]

Step 1. Compound 15a-1. 7-(3-Hydroxypropyl)-3-(4-methoxybenzyl)-3,7-dihydro-3,7,11-triazabenzo[c]fluoren-4-one. A mixture of 3-(4-methoxybenzyl)-3,7-dihydro-3,7,11-triazabenzo[c]fluoren-4-one (Compound 10c-6, 370 mg, 1.04 mmol), 3-iodopropanol (130 μL, 1.35 mmol) and cesium carbonate (678 mg, 2.08 mmol) in N,N-dimethylformamide (4.0 mL) was stirred at room temperature for 1 h. The reaction mixture was evaporated and the residue was taken up in chloroform (15 mL). The organic layer was washed with water (10 mL), dried over sodium sulfate and evaporated. The residue was purified by column chromatography eluting with chloroform:methanol (99:1→98:2) to give the title compound (328 mg, 0.793 mmol, 76%) as an off-white powder. LCMS: 86%, Rt 1.482, ESMS m/z 414 (M+H)⁺. The product was used in the next step without further purification.

Step 2. Compound 15b-1. 3-[3-(4-Methoxybenzyl)-4-oxo-3,4-dihydro-3,7,11-triazabenzo[c]fluoren-7-yl]-propionaldehyde. To a mixture of pyridine (64 μL, 0.793 mmol) and trifluoroacetic acid (30 μL, 0.397 mmol) in benzene (18 mL) was added a solution of 7-(3-hydroxypropyl)-3-(4-methoxybenzyl)-3,7-dihydro-3,7,11-triazabenzo[c]fluoren-4-one (Compound 15a-1, 328 mg, 0.793 mmol) and N,N′-dicyclohexylcarbodiimide (491 mg, 2.38 mmol) in dimethyl sulfoxide (6.0 mL). The reaction mixture was stirred at room temperature for 1 h. To this mixture was added oxalic acid (286 mg, 3.17 mmol) in a mixture of diethyl ether (6.0 mL) and methanol (6.0 mL) and the reaction mixture was stirred at room temperature for 30 min. The mixture was diluted with water (18 mL) and the layers were separated. The organic layer was dried over sodium sulfate and evaporated. The residue was purified by column chromatography eluting with chloroform:methanol (99:1) to give the title compound (216 mg, 0.526 mmol, 66%) as an off-white powder. LCMS: >35%, Rt 1.545, ESMS m/z 412 (M+H)⁺.

Step 3. Compound 15c-1. 7-(3,3-Difluoropropyl)-3-(4-methoxybenzyl)-3,7-dihydro-3,7,11-triazabenzo[c]fluoren-4-one. To a solution of 3-[3-(4-methoxybenzyl)-4-oxo-3,4-dihydro-3,7,11-triazabenzo[c]fluoren-7-yl]-propionaldehyde (Compound 15b-1, 216 mg, 0.525 mmol) in dichloromethane (6.5 mL) at −20° C. was added diethylaminosulffir trifluoride (69 μL, 0.525 mmol) and the mixture stirred at −20° C. for 2 h. The mixture was evaporated and the residue purified by column chromatography eluting with dichloromethane to give the title compound (80 mg, 0.185 mmol, 35%) as an off-white solid. LCMS: 85%, Rt 1.725, ESMS m/z 434 (M+H)⁺.

Step 4. Compound 15-1. 7-(3,3-Difluoropropyl)-3,7-dihydro-3,7,11-triazabenzo[c]fluoren-4-one. A solution of 7-(3,3-difluoropropyl)-3-(4-methoxybenzyl)-3,7-dihydro-3,7,11-triazabenzo[c]fluoren-4-one (Compound 15c-1, 70 mg, 0.162 mmol) in trifluoroacetic acid (2.0 mL) was irradiated in a microwave reactor at 150° C. for 30 h. The reaction mixture was evaporated and the residue was taken up in dichloromethane (10 mL). The organic layer was washed with 1N sodium hydroxide (4 mL), dried over sodium sulfate and evaporated. The crude product was purified by preparative HPLC to afford the title compound (8 mg, 0.026 mmol, 15%) as a white powder. LCMS: 87%, Rt 1.318, ESMS m/z 314 (M+H)⁺; ¹H NMR (500 MHz, DMSO) δ 11.31 (s, 1H), 8.62 (dd, J=4.5, 1.0 Hz, 1H), 8.35 (d, J=8.5, Hz, 1H), 8.17 (dd, J=8.0, 1.0 Hz, 1H), 8.08 (d, J=7.0 Hz, 1H), 7.82 (d, J=8.0 Hz, 1H), 7.48-7.55 (m, 1H), 7.43 (t, J=6.5 Hz, 1H), 6.19 (tt, J=56.1, 4.3 Hz, 1H), 4.71 (t, J=6.9 Hz, 2H), 2.33-2.48 (m, 2H).

### EXAMPLE 16

### Compound 16-1. 7-(3-Fluoro-propyl)-3-methyl-3,7-dihydro-3,7,11-triaza-benzo[c]fluoren-4-one

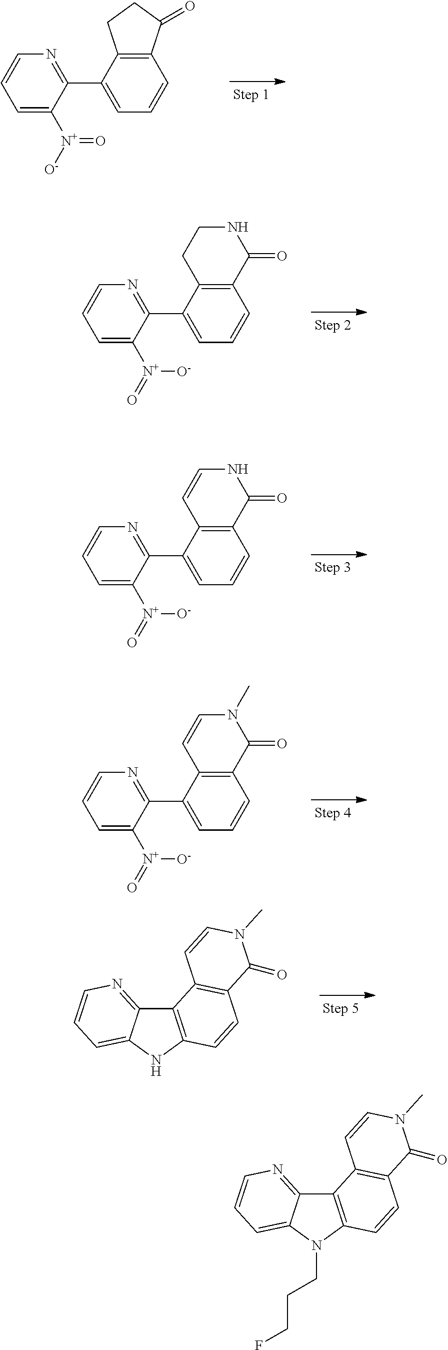 [SMILES: O=C1CCc2c1cccc2-c1ncccc1[N+](=O)[O-] ; O=C1NCCc2c1cccc2-c1ncccc1[N+](=O)[O-] ; O=c1[nH]ccc2c(-c3ncccc3[N+](=O)[O-])cccc12 ; Cn1ccc2c(-c3ncccc3[N+](=O)[O-])cccc2c1=O ; Cn1ccc2c(ccc3[nH]c4cccnc4c32)c1=O ; Cn1ccc2c(ccc3c2c2ncccc2n3CCCF)c1=O]

Step 1. Compound 16a-1. 5-(3-Nitropyridin-2-yl)-3,4-dihydro-2H-isoquinolin-1-one. To a mixture of 4-(3-nitropyridin-2-yl)-indan-1-one (Compound 10b-1, 224 mg, 0.880 mmol) and methanesulfonic acid (940 μL, 16.4 mmol) in dichloromethane (9.0 mL) at 0° C. was added sodium azide (114 mg, 1.76 mmol) in portions. The mixture was stirred at 0° C. for 1 h and then at room temperature for 20 h. The reaction mixture was poured into 20% aqueous sodium hydroxide (40 mL) and extracted with dichloromethane (3×20 mL). The combined organic layers were washed with water (1×20 mL), dried over sodium sulfate and evaporated to give the title compound (236 mg, 0.877 mmol, ca. 100%) as a tan powder. The resulting product was used in the next step without further purification. LCMS: 86%, Rt 1.069, ESMS m/z 270 (M+H)⁺.

Step 2. Compound 16b-1. 5-(3-Nitropyridin-2-yl)-2H-isoquinolin-1-one. A mixture of 5-(3-nitropyridin-2-yl)-3,4-dihydro-2H-isoquinolin-1-one (Compound 16a-1, 236 mg, 0.877 mmol) and 2,3-dicyano-5,6-dichloro-1,4-benzoquinone (400 mg, 1.760 mmol) in 1,4-dioxane (4.2 mL) was stirred at 100° C. for 7 d. The reaction mixture was diluted with 10% aqueous sodium hydroxide (10 mL) and extracted with dichloromethane (5×10 mL). The combined organic layers were dried over sodium sulfate and evaporated. The residue was purified by column chromatography eluting with chloroform:methanol (99:1→92:8) to give the title compound (66 mg, 0.247 mmol, 28%) as a yellow powder. LCMS: 90%, Rt 1.077, ESMS m/z 268 (M+H)⁺.

Step 3. Compound 16c-1. 2-Methyl-5-(3-nitropyridin-2-yl)-2H-isoquinolin-1-one. A mixture of 5-(3-nitropyridin-2-yl)-2H-isoquinolin-1-one (Compound 16b-1, 66 mg, 0.247 mmol), iodomethane (31 μL, 0.494 mmol) and cesium carbonate (163 mg, 0.494 mmol) in acetonitrile (2.5 mL) was stirred at 60° C. for 6 h. The reaction mixture was evaporated and the residue was purified by column chromatography eluting with chloroform:ethyl acetate (1:1) to give the title compound (30 mg, 0.107 mmol, 43%) as a yellow oil. LCMS: 93%, Rt 1.226, ESMS m/z 282 (M+H)⁺.

Step 4. Compound 16d-1. 3-Methyl-3,7-dihydro-3,7,11-triazabenzo[c]fluoren-4-one. A mixture of 2-methyl-5-(3-nitropyridin-2-yl)-2H-isoquinolin-1-one (Compound 16c-1, 30 mg, 0.107 mmol) and triphenylphosphine (70 mg, 0.267 mmol) in chlorobenzene (600 μL) was irradiated at 200° C. for 2 h in a microwave reactor. The mixture was evaporated and the resulting crude material was used in the next step without purification.

Step 5. Compound 16-1. 7-(3-Fluoropropyl)-3-methyl-3,7-dihydro-3,7,11-triazabenzo[c]fluoren-4-one. A mixture of 3-methyl-3,7-dihydro-3,7,11-triazabenzo[c]fluoren-4-one (Compound 16d-1, 26 mg, 0.104 mmol), 1-iodo-3-fluoropropane (20 μL, 0.208 mmol) and cesium carbonate (65 mg, 0.208 mmol) in N,N-dimethylacetamide (500 μL) was stirred at 80° C. for 2 h. The reaction mixture was evaporated and the residue was purified by column chromatography eluting with chloroform:ethyl acetate (1:1) to give the title compound (4 mg, 0.013 mmol, 13%) as a yellow powder. LCMS: 92%, Rt 1.339, ESMS m/z 310 (M+H)⁺. ¹H NMR (500 MHz, CDCl₃) δ 8.71 (dd, J=4.6, 1.1 Hz, 1H), 8.64 (d, J=8.9 Hz, 1H), 8.26 (d, J=7.3 Hz, 1H), 7.85 (d, J=8.2 Hz, 1H), 7.60 (d, J=8.9 Hz, 1H), 7.40-7.45 (m, 1H), 7.37 (d, J=7.3 Hz, 1H), 4.60 (t, J=6.6 Hz, 2H), 4.41 (dt, J=47, 5.0 Hz, 2H), 3.72 (s, 3H), 2.31 (dquint, J=27.5, 6.0 Hz, 2H).

### EXAMPLE 17

This example provides a table (below) of the compounds of the present disclosure and their activity towards four different cell lines (i.e., CWR22R, Hela, PC3, and MDA-MB-231). The IC50 for inhibition of growth is divided into 4 categories: A<1 μM, B 1-5 μM, 5-20 μM, D>20 μM.

| | | | | MDA-MB- |
|---|---|---|---|---|
| Compound | CWR22R | Hela | PC3 | 231 |
| 1-1 | A | D | D | D |
| 1-3 | C | C | C | C |
| 1-4 | A | D | D | D |
| 1-5 | A | D | D | D |
| 1-6 | A | D | D | D |
| 1-7 | A | D | D | D |
| 1-8 | A | D | D | D |
| 1-9 | A | D | D | D |
| 1-10 | A | D | D | D |
| 1-11 | A | D | D | D |
| 1-12 | A | D | D | D |
| 1-14 | A | D | D | D |
| 1-15 | B | D | D | D |
| 1-16 | B | D | D | D |
| 2-1 | A | D | D | D |
| 2-2 | A | D | D | D |
| 2-3 | B | C | D | C |
| 2-4 | A | C | C | D |
| 2-5 | A | D | D | D |
| 3-1 | A | D | D | D |
| 3-2 | A | D | D | D |
| 3-3 | A | D | D | C |
| 4-1 | A | B | D | A |
| 4-2 | A | C | D | B |
| 4-3 | C | D | D | D |
| 4-4 | C | C | C | C |
| 5-1 | A | D | | D |
| 5-2 | A | D | D | D |
| 5-3 | A | D | D | D |
| 6-1 | A | D | D | D |
| 7-1 | A | C | C | C |
| 7-2 | B | D | D | D |
| 8-1 | A | D | D | D |
| 8-2 | A | C | D | C |
| 8-3 | B | D | D | D |
| 8-4 | C | D | D | D |
| 10-1 | B | D | C | C |
| 10-2 | A | D | C | C |
| 10-5 | B | D | D | D |
| 10-6 | A | D | D | D |
| 10-7 | A | D | D | C |
| 10-9 | A | D | D | D |
| 10-10 | A | D | D | D |
| 11-1 | C | D | D | D |
| 11-2 | A | D | D | D |
| 11-3 | B | D | D | D |
| 12-1 | C | D | D | D |
| 13-1 | A | D | D | D |
| 13-2 | C | D | D | D |
| 14-1 | A | D | D | C |
| 15-1 | D | D | D | D |

### EXAMPLE 18

This example shows various cancer cell lines and their sensitivity towards compounds of the present disclosure represented by c52 (S represents a cell line sensitive to growth inhibition, R represents a resistant cell line).).

| Cell line | Description | c52 sensitivity |
|---|---|---|
| LNCaP | castration resistant prostate cancer, AR+ | S |
| C4-2 | | S |
| CWR22R | | S |
| VCaP | castration resistant prostate cancer, Armut | R |
| PC3 | castration resistant prostate cancer, AR− | R |
| Du145 | | R |
| PPC1 | | R |
| Hela | cervix adenocarcinoma, AR− | R |
| HT1080 | fibrosarcoma AR− | R |
| MRC5 | normal fibroblasts, AR− | R |
| PANC1 | pancreatic adenocarcinoma, AR− | R |
| MiaPaca | | R |
| RCC45 | renal cell carcinoma, AR− | R |
| SKRC45 | | R |
| ACHN | | R |
| NKE | normal kidney, AR− | R |
| HepG2 | hepatocellular carcinoma, AR+ | S |
| Hep3B | | S |
| normal hepatocytes | | R |
| HMEC | normal breast | R |
| MCF10A | mammary gland epithelium | R |
| AU565 | Luminal breast carcinoma, AR+ | S |
| ZR7530 | | S |
| ZR751 | | S |
| BT474 | | S |
| MDAMB415 | | S |
| MDAMB453 | | S |
| T47D | | S |
| MCF7 | | S |
| SUM185PE | | S |
| HCC1419 | | R |
| UACC893 | | R |
| CAMA1 | | R |
| HCC202 | Luminal breast carcinoma, AR− | S |
| EFM192A | | S |
| SKBR3 | | S |
| UACC812 | | S |
| LY2 | | S |
| ZR75B | | R |
| EFM192B | | R |
| JIMT1 | | R |
| HCC1187 | Basal breast carcinoma, AR+ | S |
| BT549 | | R |
| MDAMB468 | Basal breast carcinoma, AR− | S |
| HCC3153 | | S |
| SUM149PT | | S |
| HCC1143 | | R |
| 21MT1 | | R |
| 21MT2 | | R |
| 21NT | | R |
| MDAMB231 | | R |
| MDAMB361 | breast carcinoma of unspecified type | S |
| HCC1569 | | S |
| 184B5 | | S |
| HCC1395 | | S |
| MDAMB157 | | S |
| EVSA1 | | S |
| HCC1500 | | R |
| MDAMB436 | | R |
| 21PT | | R |
| CAL85-1 | | R |
| Mx1 | | R |
| CAL148 | | R |
| MCF-10F | | R |

### EXAMPLE 19

This example provides compounds of the present disclosure and testing of the compounds against cancers.

Structures and reference numbers for compounds in this example and Example 20 are as follows:

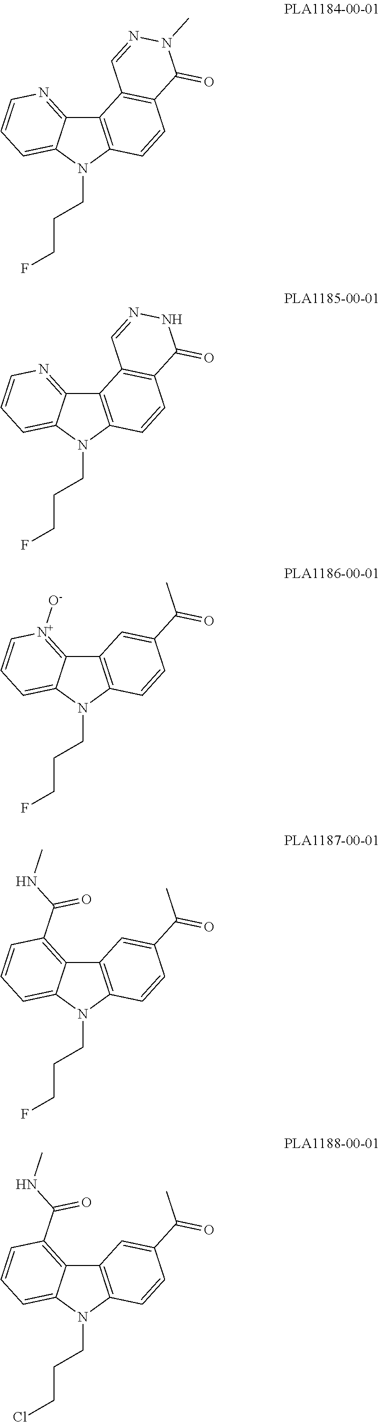

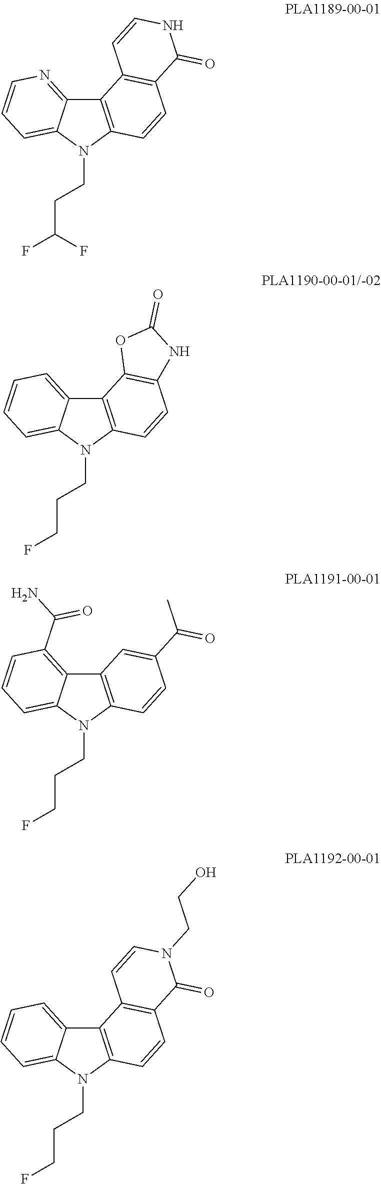

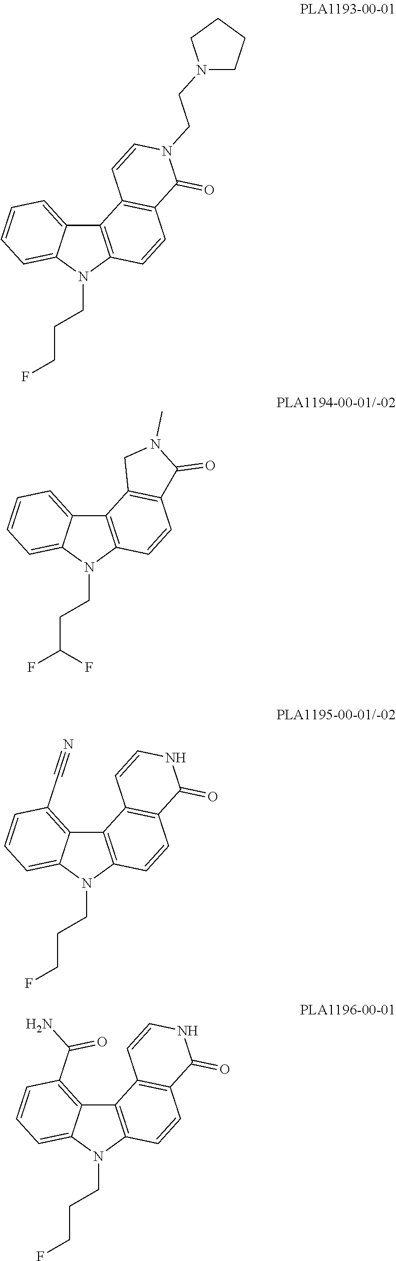

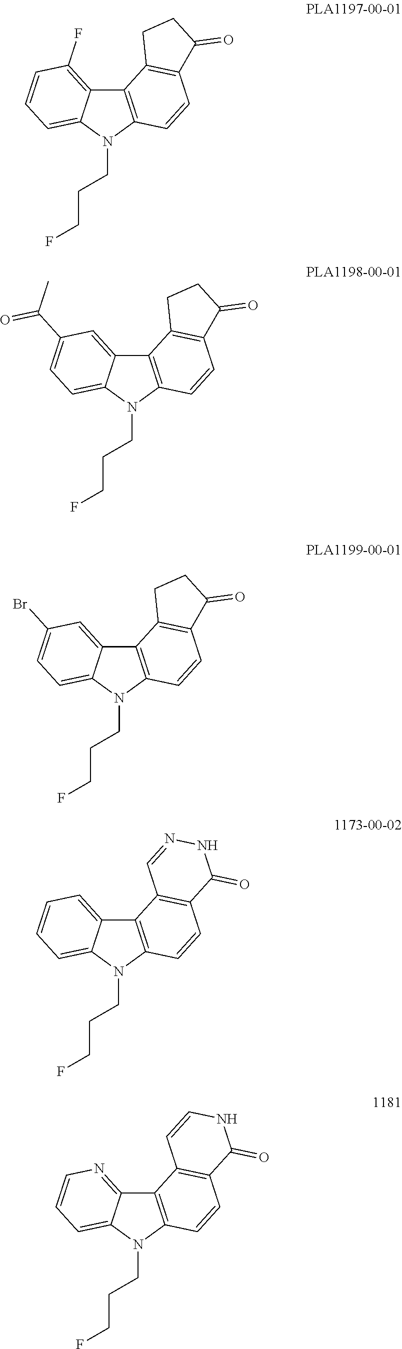

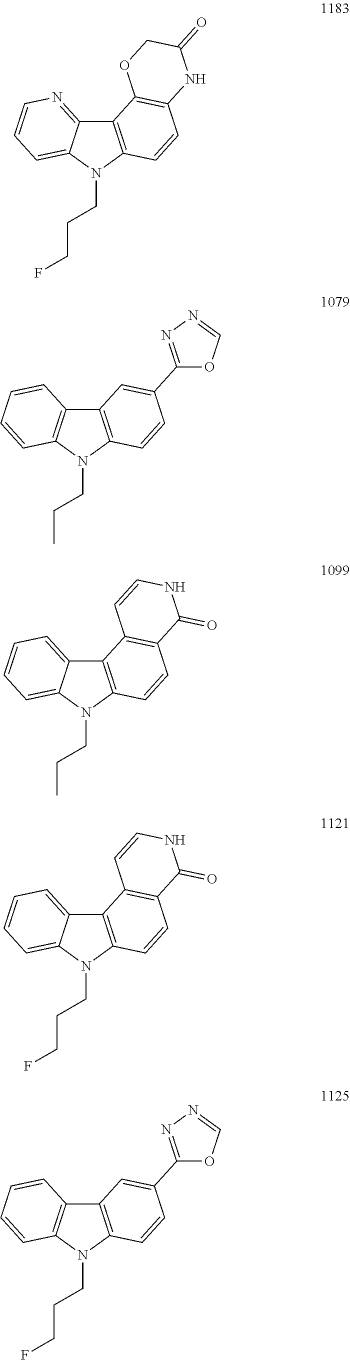 [SMILES: O=C1COc2c(ccc3c2c2ncccc2n3CCCF)N1 ; CCCn1c2ccccc2c2cc(-c3nnco3)ccc21 ; CCCn1c2ccccc2c2c3cc[nH]c(=O)c3ccc21 ; O=c1[nH]ccc2c1ccc1c2c2ccccc2n1CCCF ; FCCCn1c2ccccc2c2cc(-c3nnco3)ccc21]

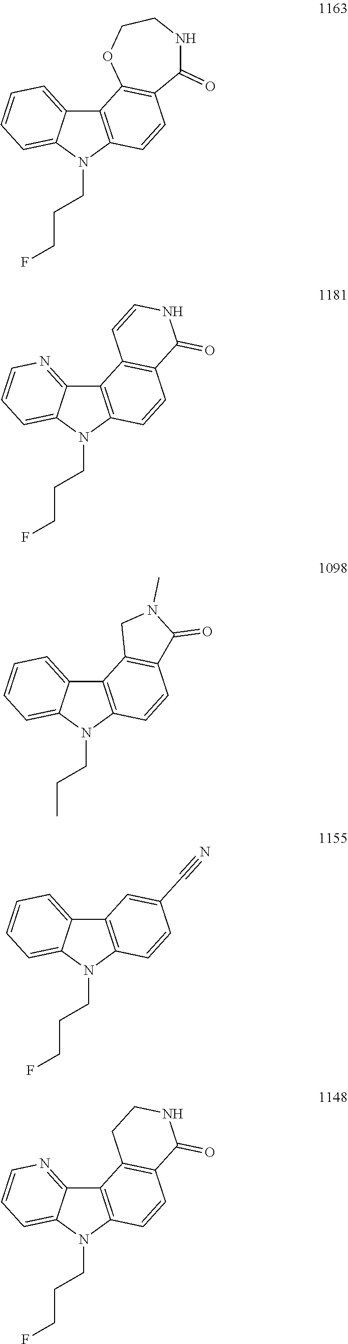 [SMILES: O=C1NCCOc2c1ccc1c2c2ccccc2n1CCCF ; O=c1[nH]ccc2c1ccc1c2c2ncccc2n1CCCF ; CCCn1c2ccccc2c2c3c(ccc21)C(=O)N(C)C3 ; N#Cc1ccc2c(c1)c1ccccc1n2CCCF ; O=C1NCCc2c1ccc1c2c2ncccc2n1CCCF]

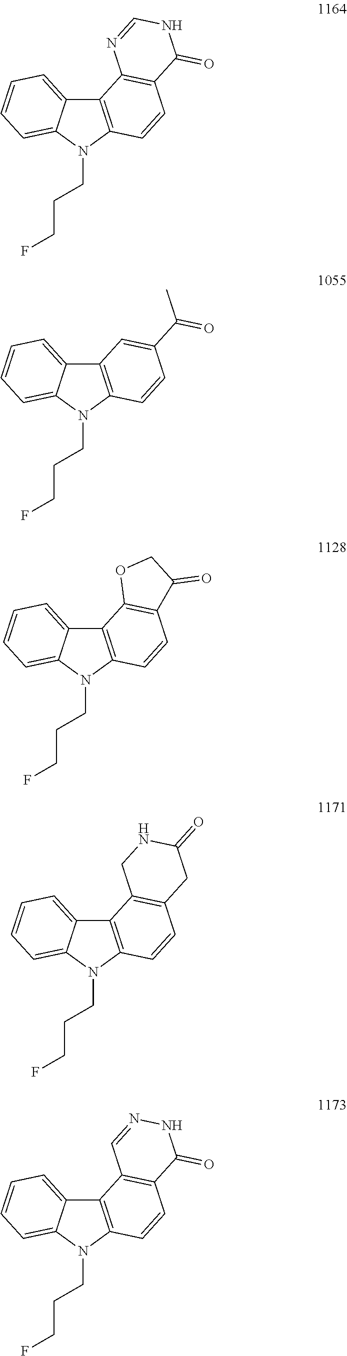 [SMILES: O=c1[nH]cnc2c1ccc1c2c2ccccc2n1CCCF ; CC(=O)c1ccc2c(c1)c1ccccc1n2CCCF ; O=C1COc2c1ccc1c2c2ccccc2n1CCCF ; O=C1Cc2ccc3c(c2CN1)c1ccccc1n3CCCF ; O=c1[nH]ncc2c1ccc1c2c2ccccc2n1CCCF]

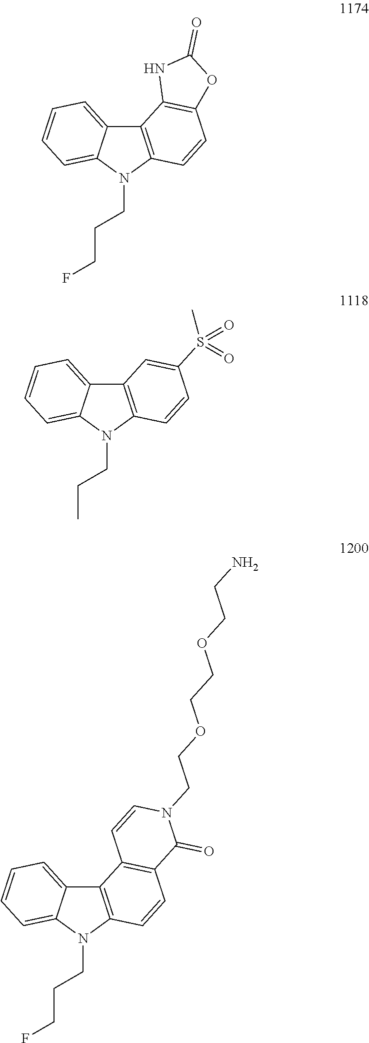 [SMILES: O=c1[nH]c2c(ccc3c2c2ccccc2n3CCCF)o1 ; CCCn1c2ccccc2c2cc(S(C)(=O)=O)ccc21 ; NCCOCCOCCn1ccc2c(ccc3c2c2ccccc2n3CCCF)c1=O]

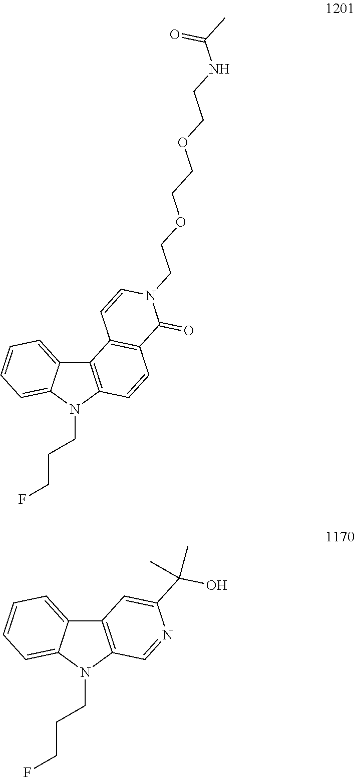 [SMILES: CC(=O)NCCOCCOCCn1ccc2c(ccc3c2c2ccccc2n3CCCF)c1=O ; CC(C)(O)c1cc2c3ccccc3n(CCCF)c2cn1]

The 4- or 5-digit reference number (where the 5-digit number has a leading 0) each refer to the same compound. Also, the reference number may have a PLA prefix or -00-01 or -00-02 suffix. For example, PLA01055, PLA1055, PLA01055-00-01, and PLA01055-00-02, PLA1055-00-01, and PLA1055-00-02 all refer to the same compound.

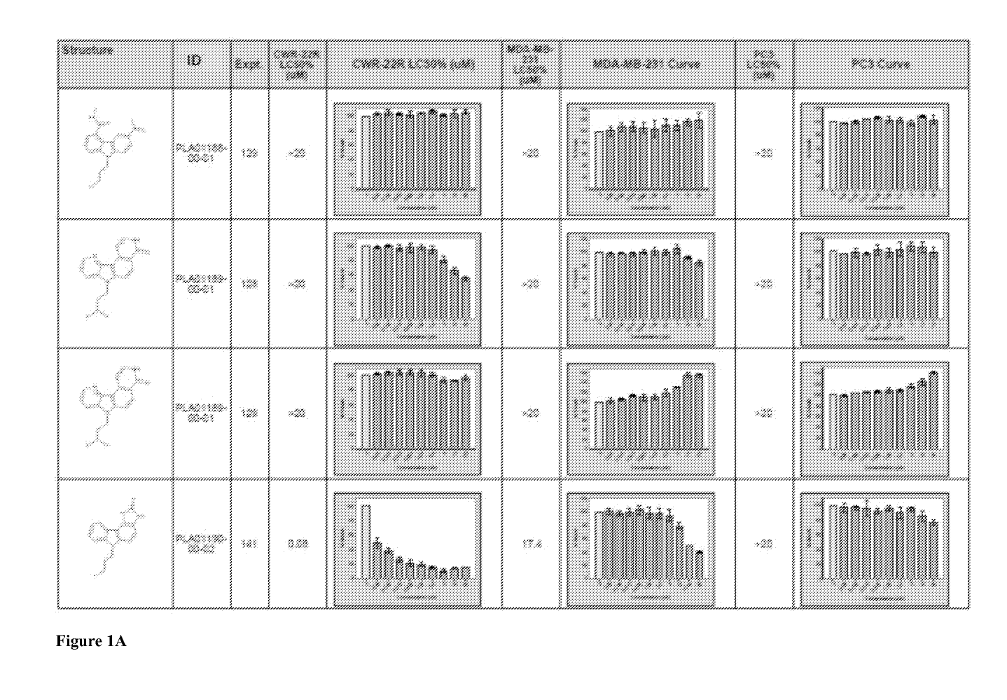

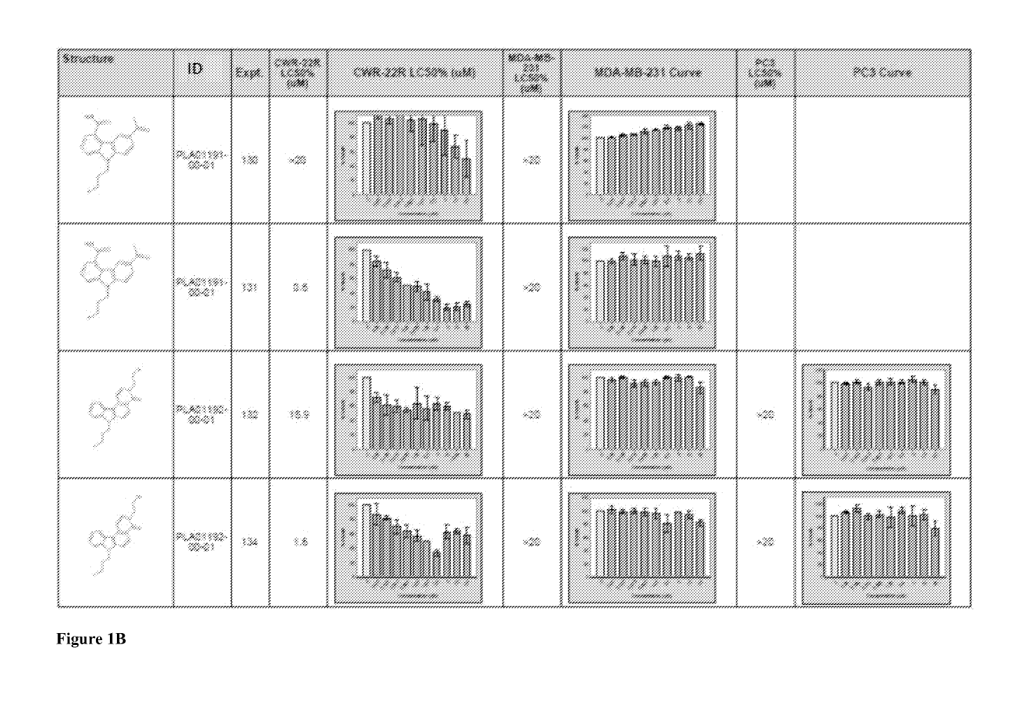

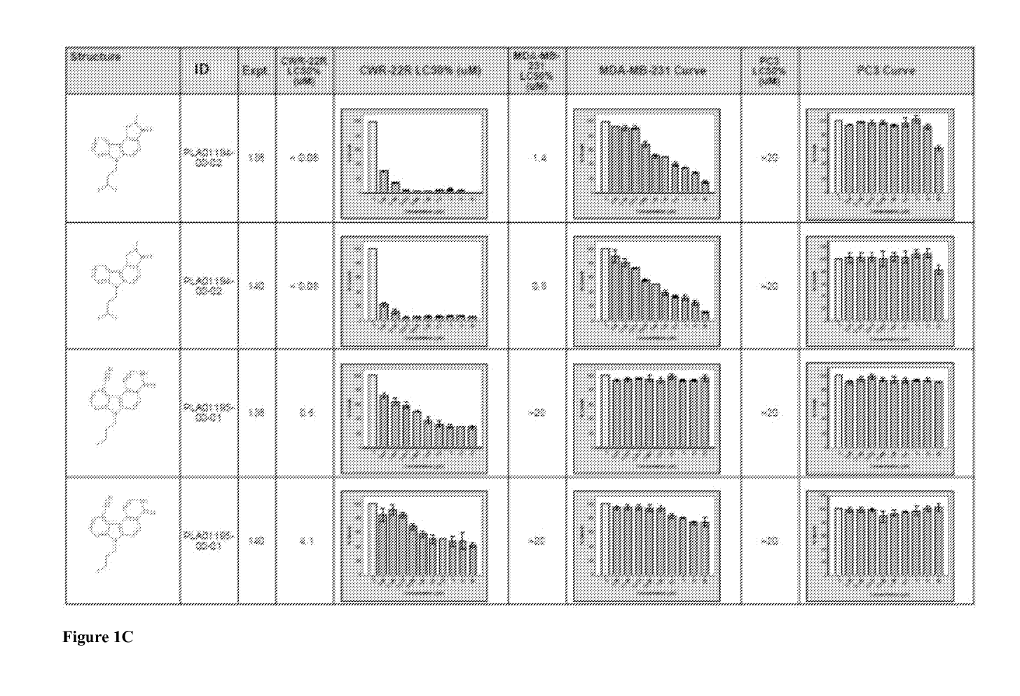

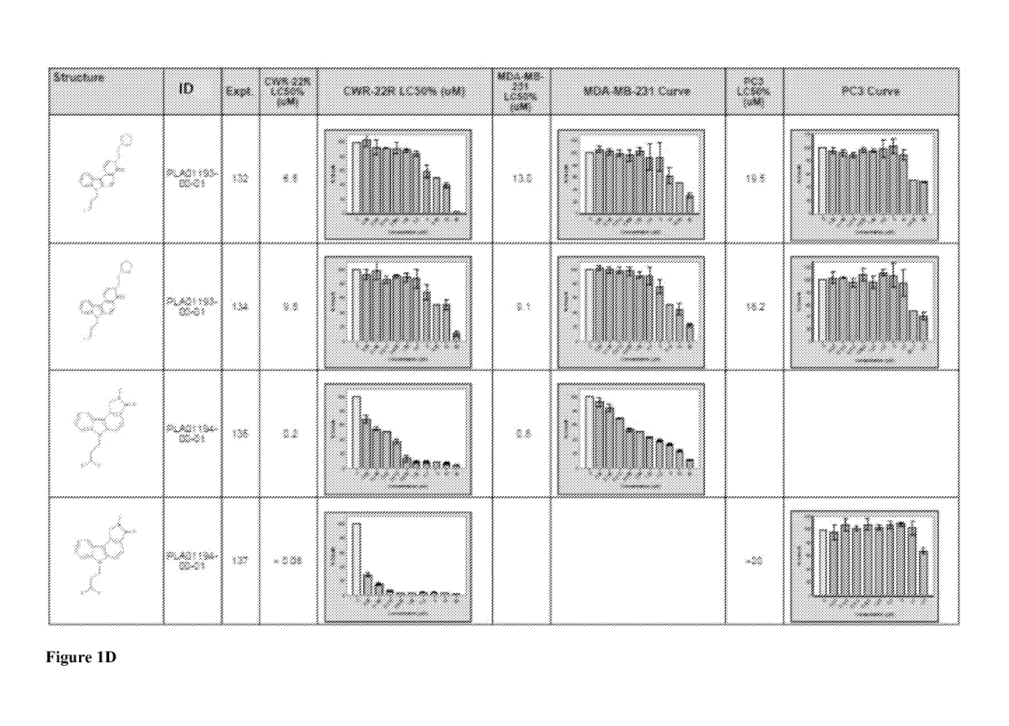

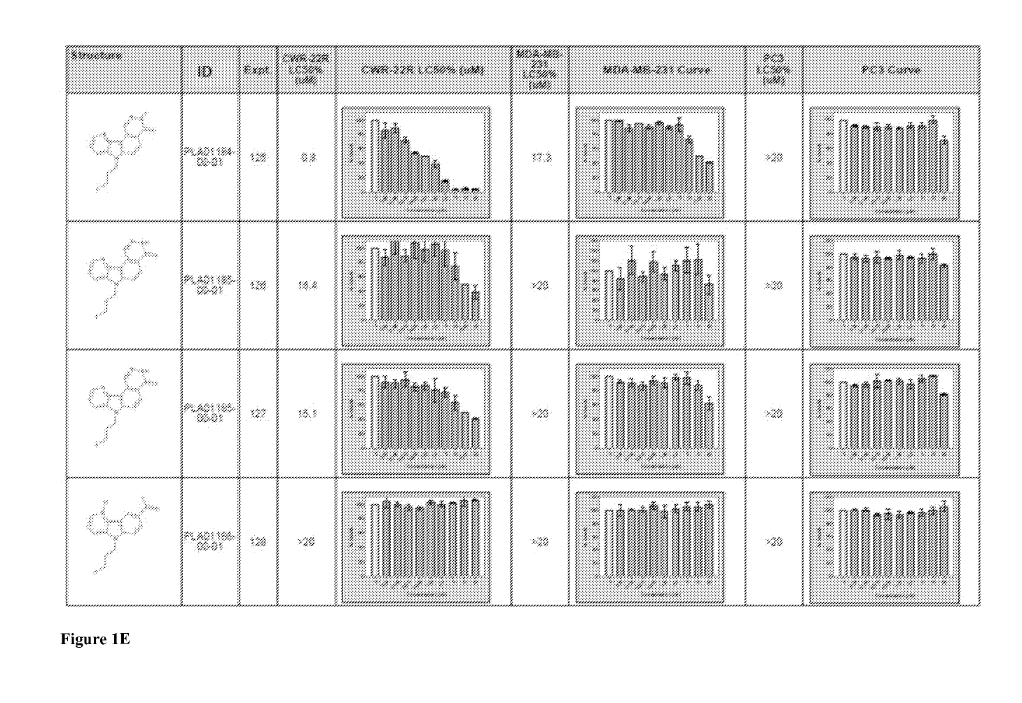

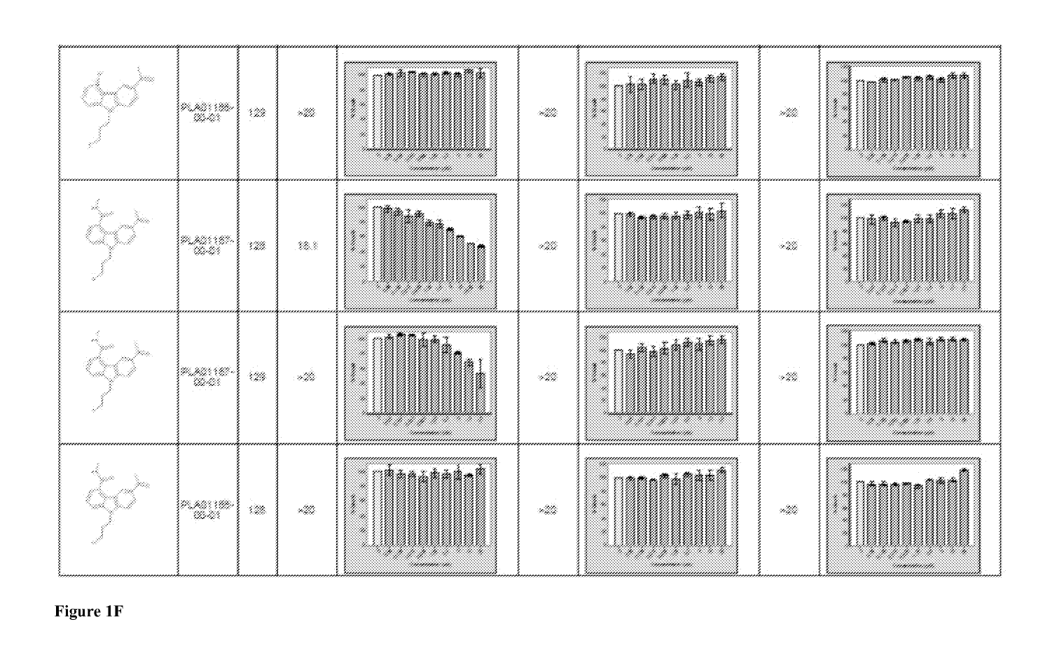

All compounds were tested for toxicity using 4 cells lines, target cells-CWR22R and non-target cells, Hela, PC3 and MDA-MB-231 (FIGS. 1A-F). Compounds with LC50 for CWR22R cells below luM and no toxicity for other three cell lines at concentrations >20uM were tested for metabolic stability and solubility (Table 1).

Table 1. Metabolic stability and solubility of the selected compounds.

TABLE 1
Metabolic stability and solubility of the selected compounds.

| | Aqueous | | Aqueous | Aqueous | Aqueous |
|---|---|---|---|---|---|
| Compound: | Solubility | | Solubility | Solubility | Solubility |
| (n = 3) | (pH 7.4) | | Classification | (pH 7.4) | Classification |
| PLA1181 | <2 | μM | Low | 2.3 μM | Low |
| PLA1183 | 8.9 | μM | Low | 12.7 μM | Moderate |
| PLA1191 | 130 | μM | High | 30% | Moderate |
| PLA1192 | 15 | μM | Moderate | 27% | Moderate |
| PLA1193 | 140 | μM | High | 3% | Unstable |
| PLA1194 | 18 | μM | Moderate | 28% | Moderate |
| PLA1190 | 3.5 | μM | Low | 77% | Stable |
| PLA1195 | 2.7 | μM | Low | 63% | Moderate |
| PLA1196 | 26 | μM | Moderate | 65% | Moderate |
| PLA1197 | 4.0 | μM | Low | 73% | Stable |
| PLA1198 | 2.5 | μM | Low | 46% | Moderate |
| PLA1199 | 2.3 | μM | Low | 42% | Moderate |

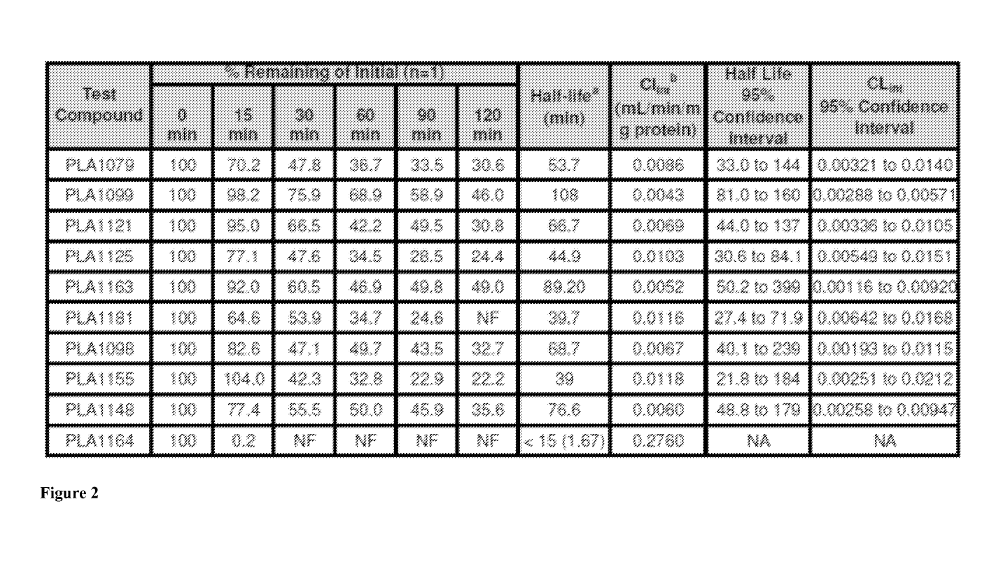

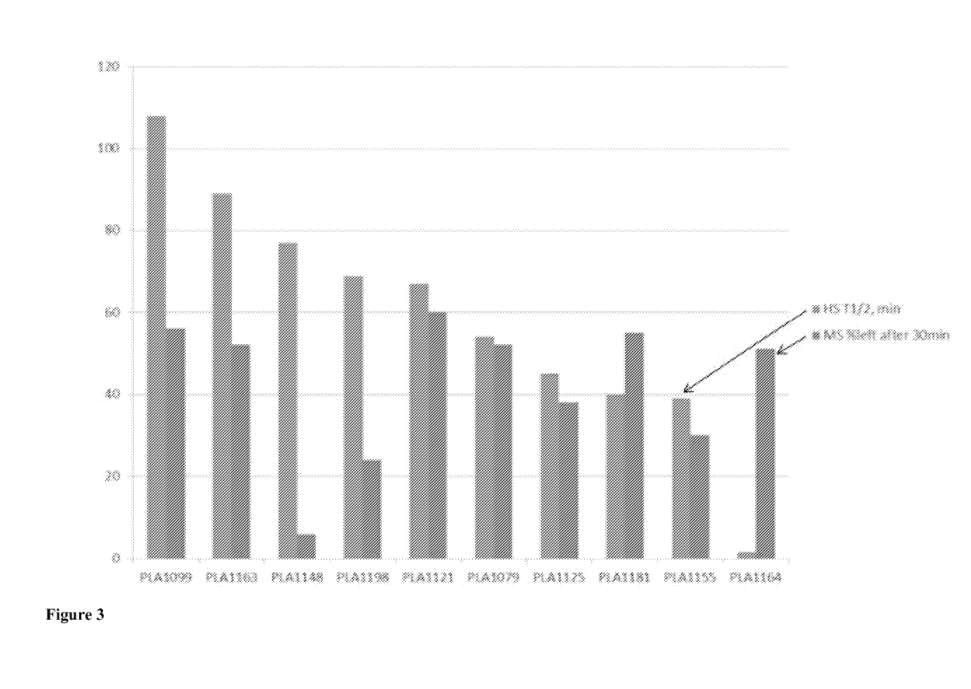

8 compounds were selected based on the solubility and metabolic stability as well as considering chemical diversity to test for time dependence stability in the presence of mouse hepatocytes (FIG. 2). Unexpectedly this assay showed week correlation with the stability of the compounds in the presence of liver microsomes (FIG. 3). Two compounds, PLA1190 and 1197, have desirable metabolic stability, 70 and 77% left after 30 minute incubation with mouse liver microsome respectively. Formulations for in vivo administration were developed for 4 compounds, PLA1079, 1125, 1098 and 1148.

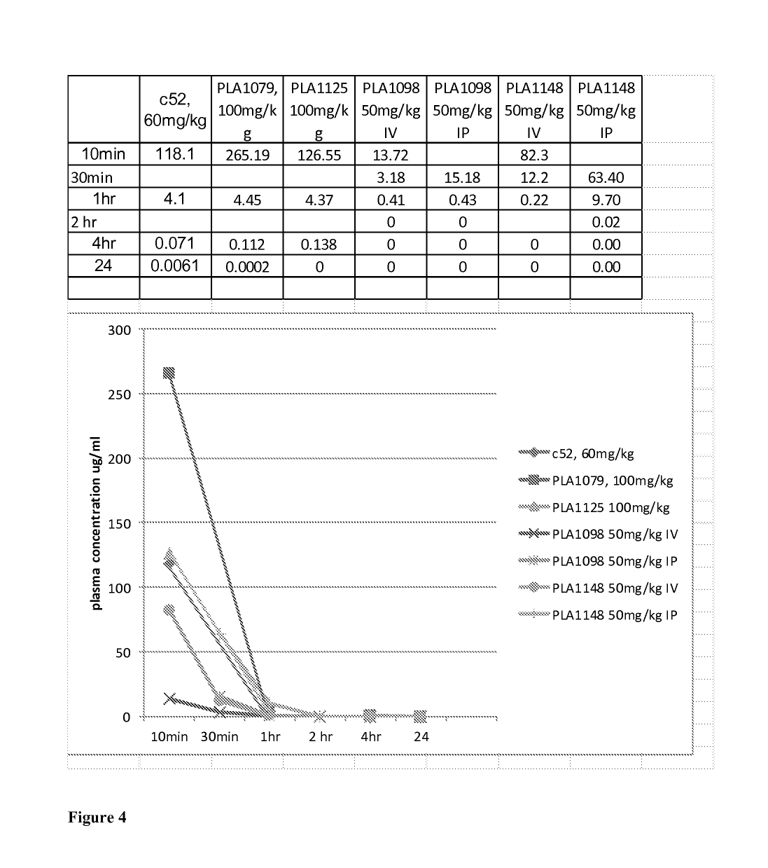

4 compounds, PLA1079, 1125, 1098 and 1148 were tested in vivo to obtain plasma concentration at different time points after intravenous and intraperitoneal injections (FIG. 4). Data were very similar for all 4 compounds, fast reduction of concentration during the first hour. Although injected doses were very high, 50-100 mg/kg they were not enough to obtain plasma concentration above LC50% in vitro at any moment, except immediately after administration (5 minutes). Positive finding was very high bioavailability of all four compounds through IP injection: plasma concentrations were similar or even higher after IP than IV administration.

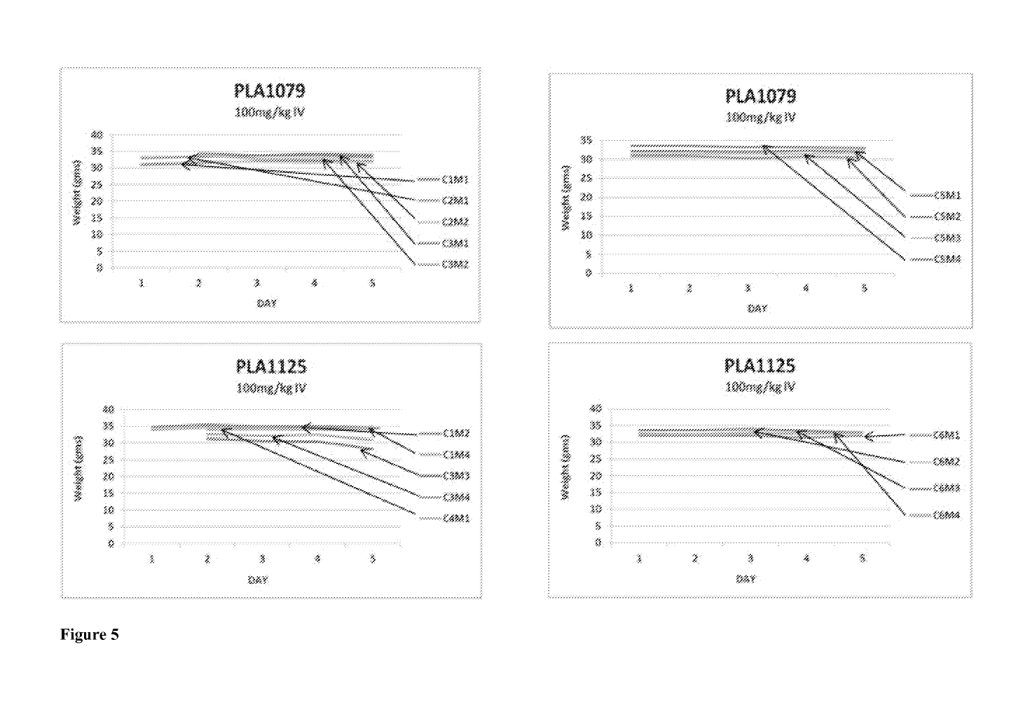

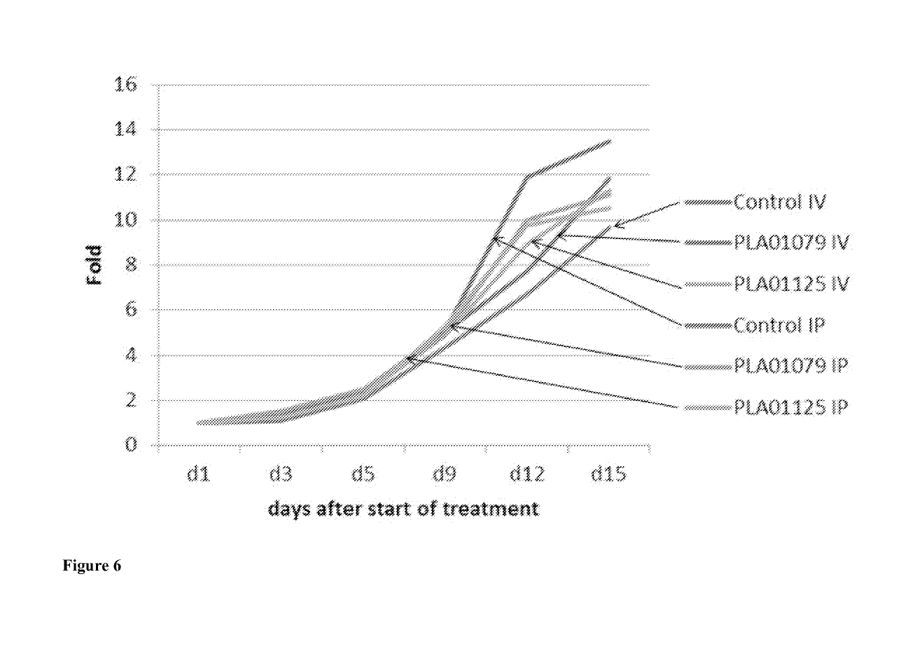

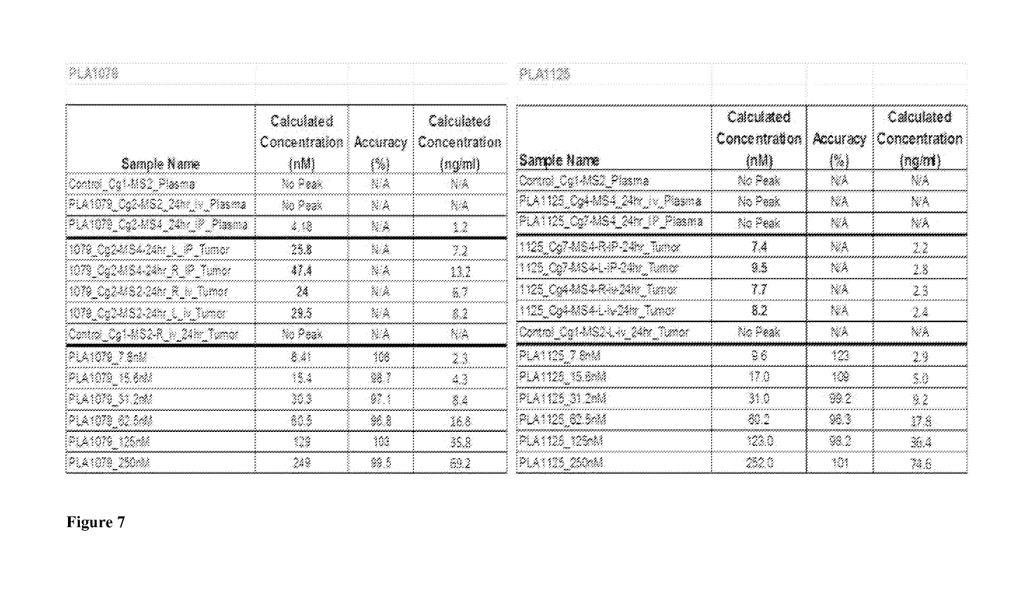

We tested these compounds in an in vivo efficacy experiment using model of CRPC in SCID mice, CWR22R. Two compounds, PLA1079 and 1125, which were synthesized in the amount of 500 mg were tested in full scale experiment with either 5 IV daily injections or 10 IP daily injections. After 5 IP or IV injections intra-tumor concentration of the compounds were measured in few mice from this experiment. It was significantly below LC50% for the same tumor cells in vitro. The absence of in vivo efficacy is attributable due to low intra-tumor concentration of the compounds (FIGS. 5, 6, and 7).

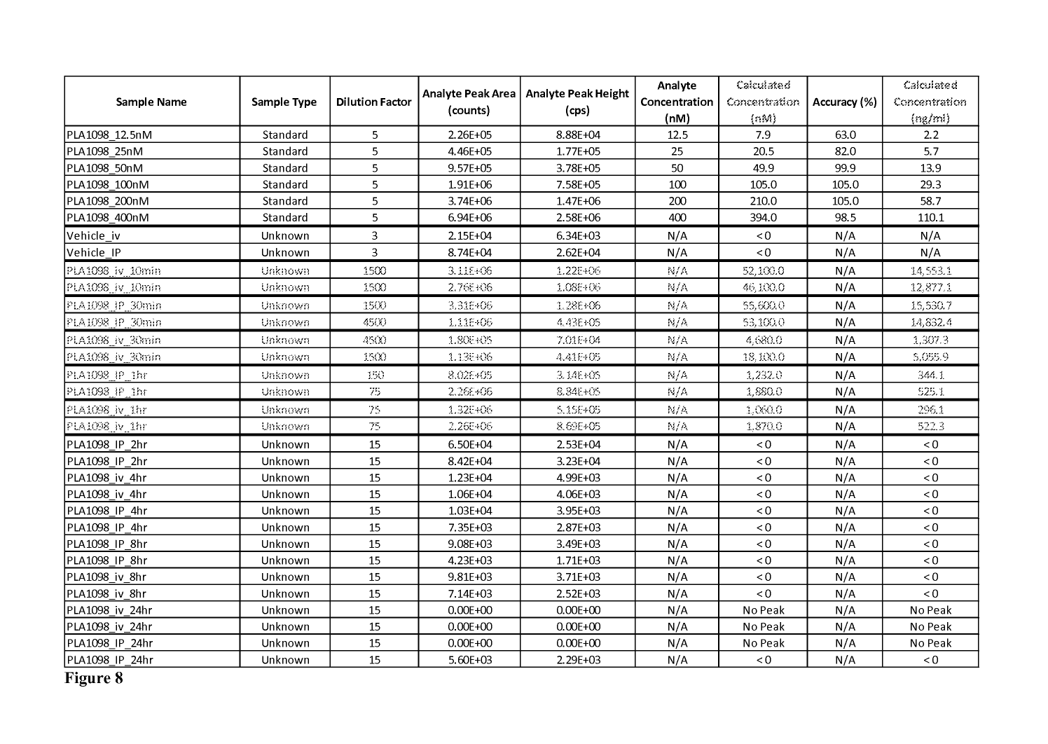

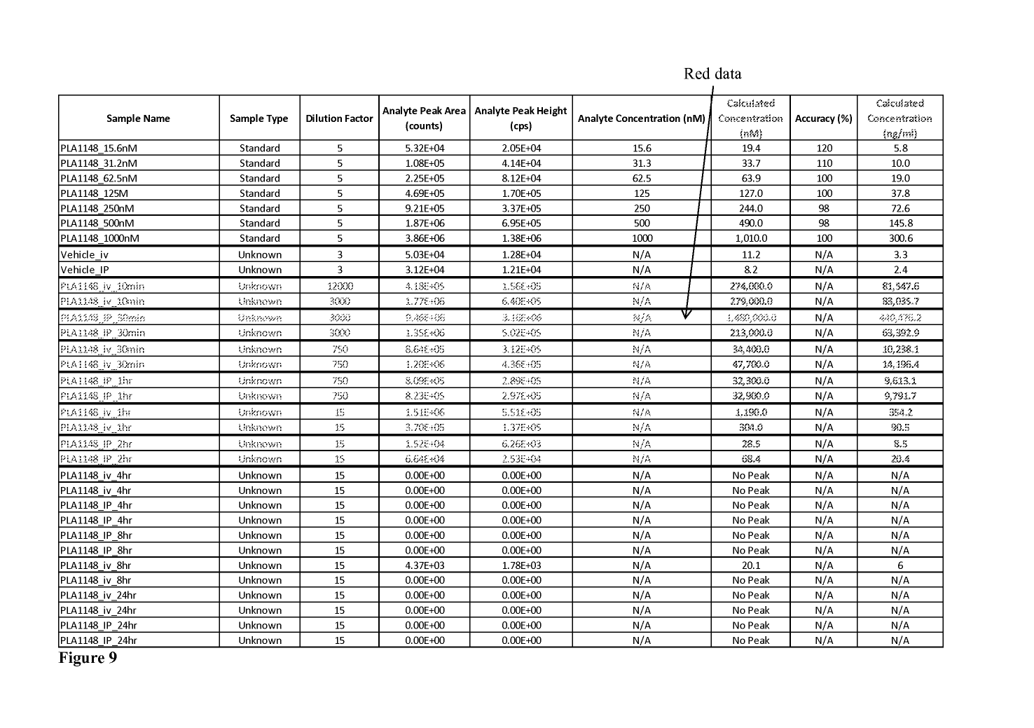

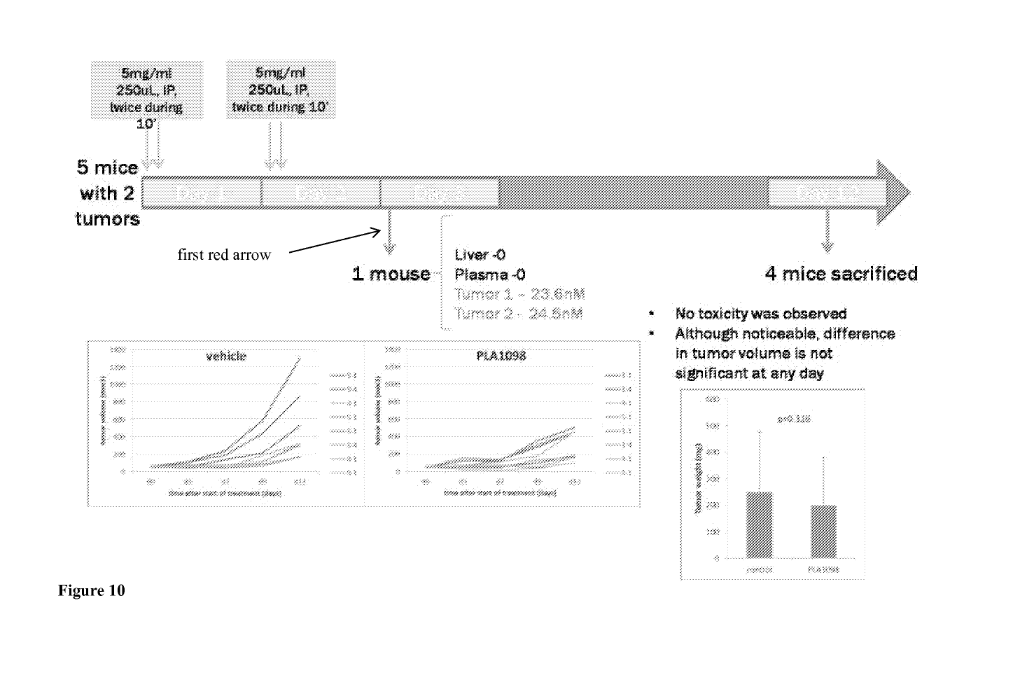

Two other compounds, PLA1148 and 1098 (PK testing in FIGS. 8 and 9) were tested in a small scale efficacy experiment. Upon PK testing of the compound PLA1148 one mouse was injected twice with 10 min interval. However the concentration of the drug in plasma of this mouse was not twice, but >4 times higher than in mice injected once. This suggests that the saturation of liver metabolizing enzymes lead to the sharp increase in the plasma concentration. It was decided to utilize this observation in an attempt to get higher plasma and tumor concentration of both compounds. Therefore in the pilot efficacy experiment in the same model of CWR22R cells mice were treated with 2 IP injections with 10 minute interval. Treatment with PLA1098 was carried out for two days. Mice were fine during the treatment, no signs of side effects were noticed and at 12 days after the treatment mice were sacrificed and tumors were excised and weighed. There were certain reduction of tumor volume and tumor weigh in the 1098 treated group vs vehicle control, but not statistically significant (FIG. 10). Short treatment period as well as small group size suggest that this difference may be significant if both conditions will be enlarged. Intratumor concentration of the compound in 2 tested tumors at 24 hours after last injection was 25 nM, what is ½ of LC50 for PLA1098 (FIG. 10). However this is so far the highest concentration found in tumor. Interestingly there were no detectable PLA1098 in plasma and liver at the same time.

Compound PLA1148 were injected only one day due to acute toxicity developed quickly after injection, symptoms suggested acute liver failure. 2 mice died next day and 3 other during next 5 days. Two mice died on the 5th day had almost undetectable tumors. There were no drug detected in one mouse at 24 hours after injections neither in tumor, nor in liver, plasma concentration was 8.5 nM. The same plasma concentration was found in the mice euthanized at 48 hours after treatment. In this mouse tumor had 84 nM of PLA1148 (LC50%˜70 nM) and liver had 49 nM. This compound may be also potentially promising.

Three more compounds, PLA1099, 1121 and 1163 were poorly soluble and therefore were tested for PK using IP injection of the drug in suspension.

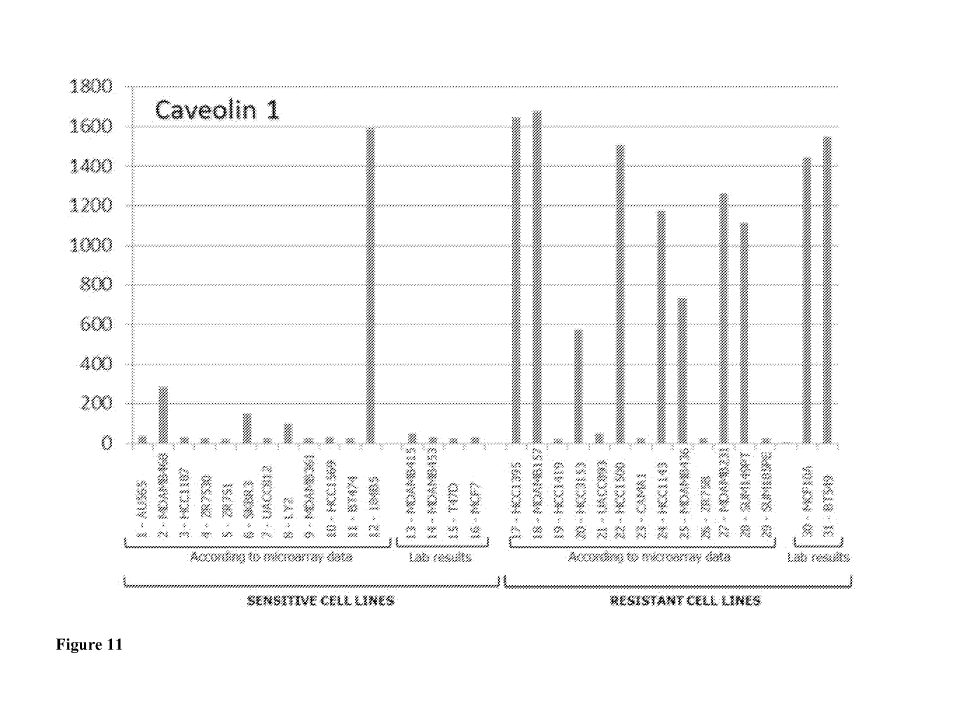

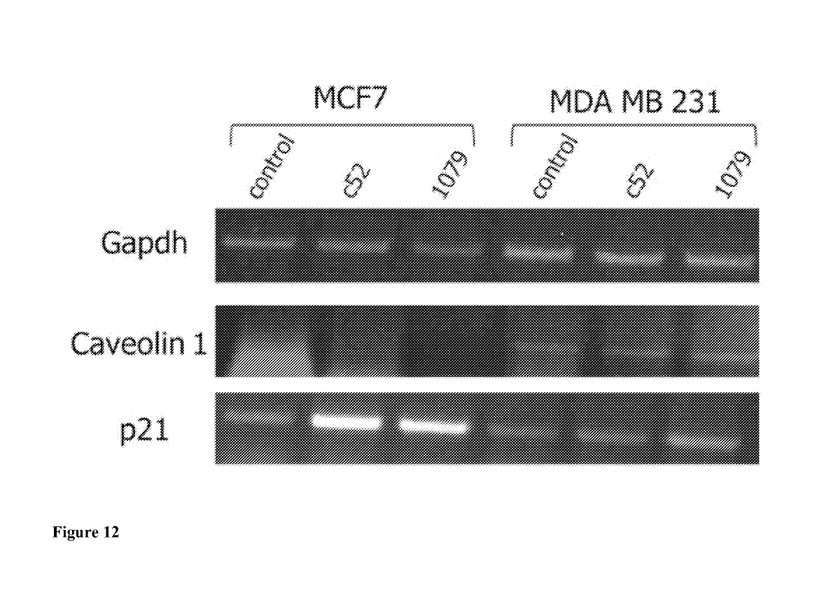

Analysis of gene expression among 50 breast cancer cell lines differed in sensitivity as well as shRNA screening of resistant breast cancer cells identified Calveolin1 (FIG. 11) as a potential gene associated with resistance to c52. mRNA expression of Calveolin1 was tested among our sensitive and resistant cells as well as in response to c52 treatment. As expected Calveolin1 was expressed only in resistant cells, however it was not changed upon c52 treatment (FIG. 12). Caveolin1 was overexpressed in resistant cell lines, while almost all of the sensitive cell lines showed low or no expression (FIG. 12). Calveolin1 was cloned in lentiviral expression vector and are now testing if overexpression of Calveolin1 in sensitive cells will make them resistant to c52 treatment.

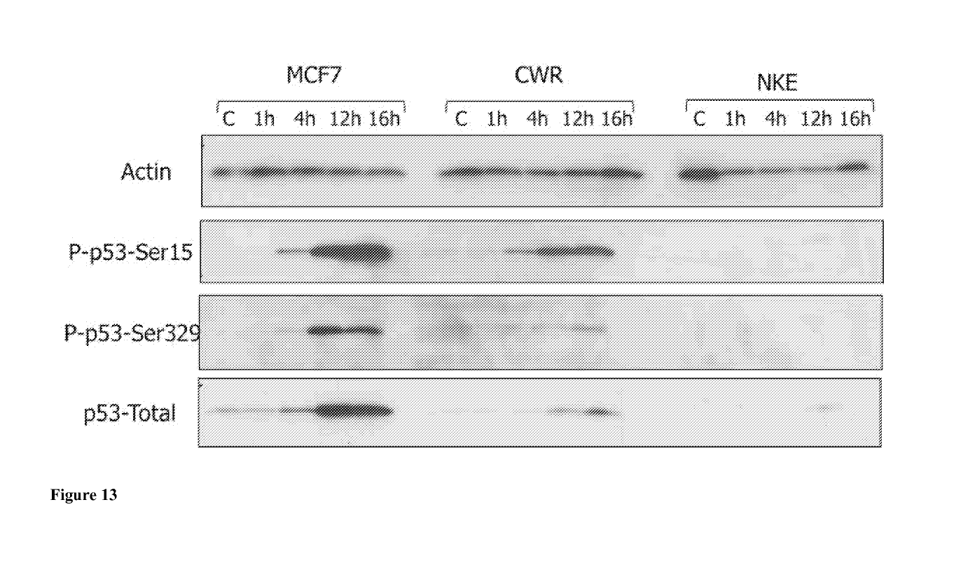

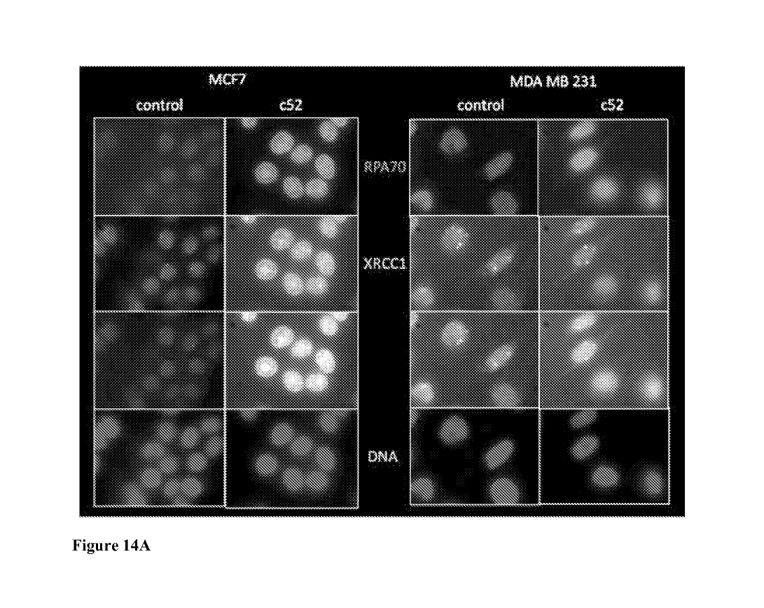

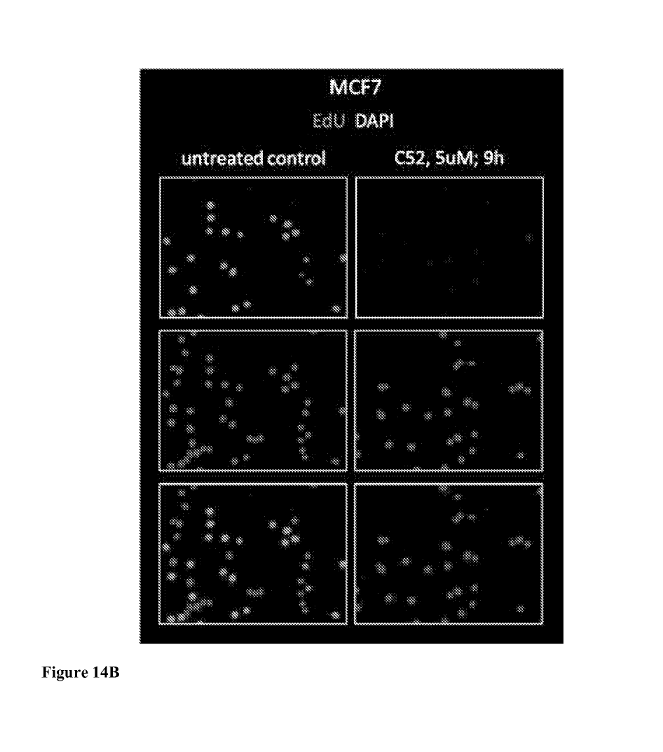

It was also found that c52 induces DNA-damage and p53 activation in sensitive, but not resistant cells (FIG. 13). Analysis of the type of DNA damage showed that it results from replication stress which c52 induces in sensitive, but not resistant cells (FIG. 14).

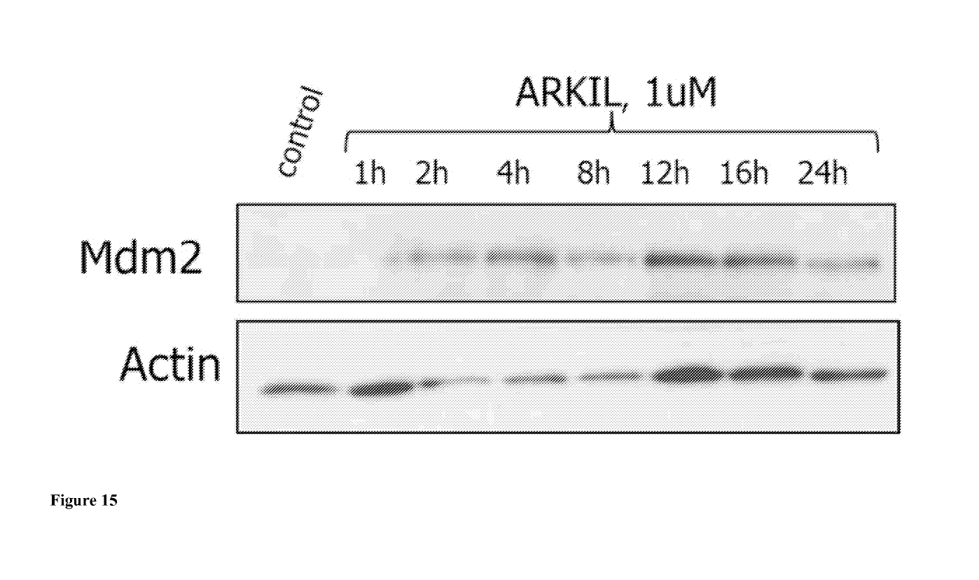

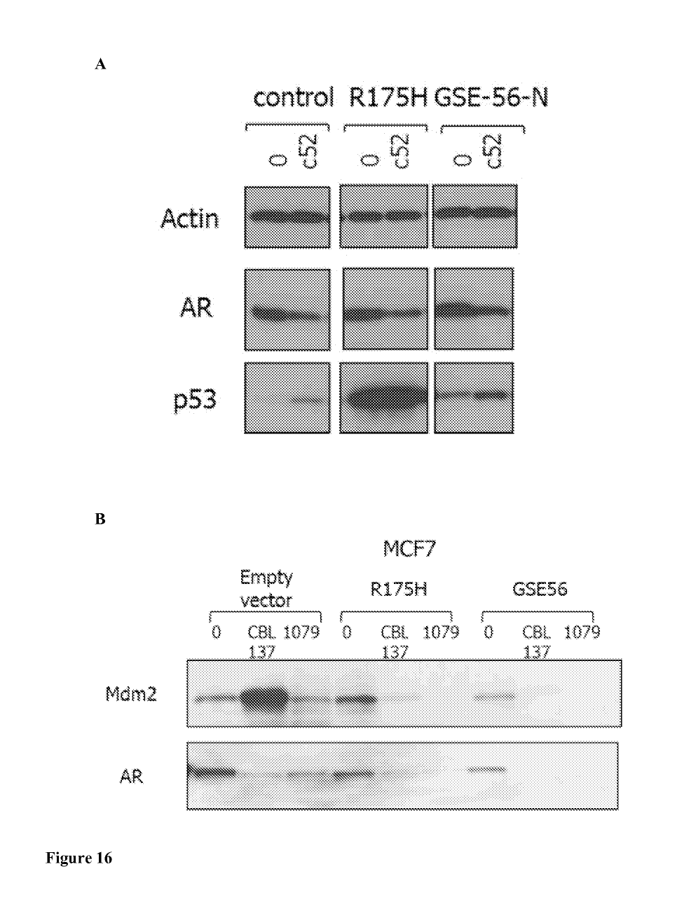

Also tested was the hypothesis that degradation of androgen receptor in prostate and breast cancer cells may be due to p53 induced accumulation of mdm2, which is also ubiquitin ligase for androgen receptor (FIG. 15). p53 was inactivated in several cell lines using different approaches what led to the blockade of mdm2 accumulation after c52 treatment. However androgen receptor was still degraded in c52 treated cells even in the absence of p53 activation and mdm2 accumulation (FIG. 16). It was concluded that c52 induces degradation of androgen receptor through p53 independent mechanism.

For target identification through affinity chromatography and sensitivity to proteolysis methods synthesis of c52 with flexible linker was carried out. PLA1098 demonstrated evidence of in vivo efficacy without any signs of toxicity.

### EXAMPLE 20

This example provides compounds of the present disclosure and testing of the compounds against cancers.

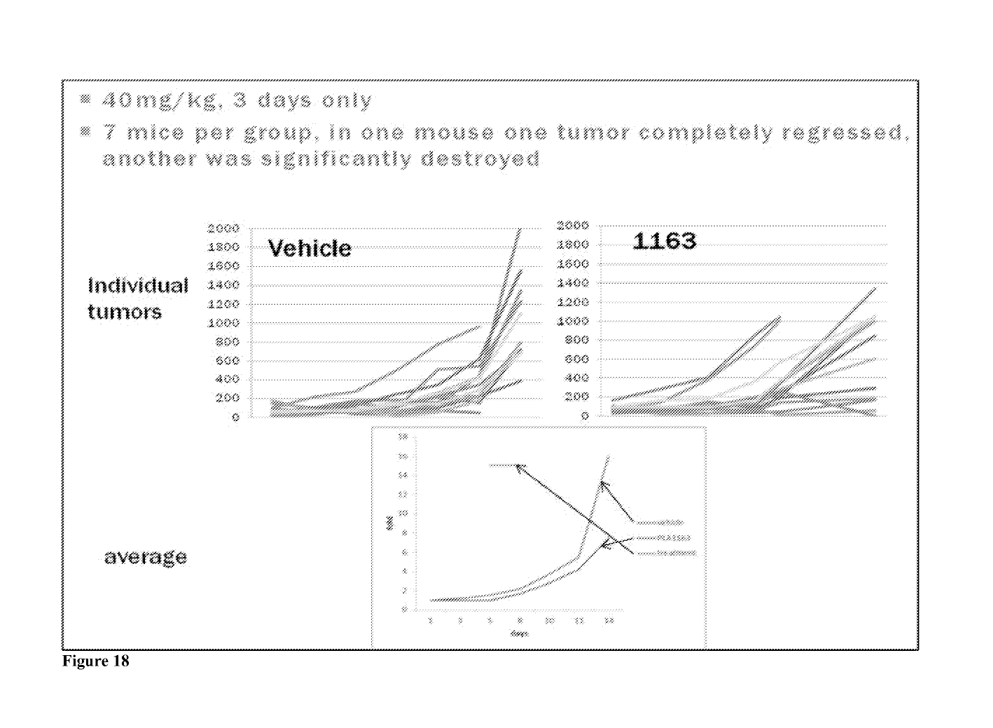

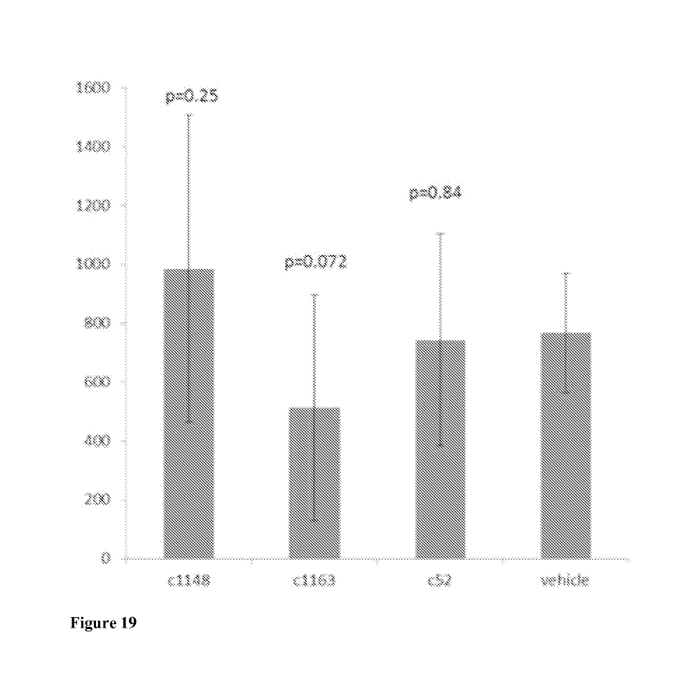

Pilot efficacy experiments were run for compounds: PLA1163, PLA1148 using the same model of subcutaneous xenografts of CWR22R cells in SCID mice (FIGS. 18 and 19).

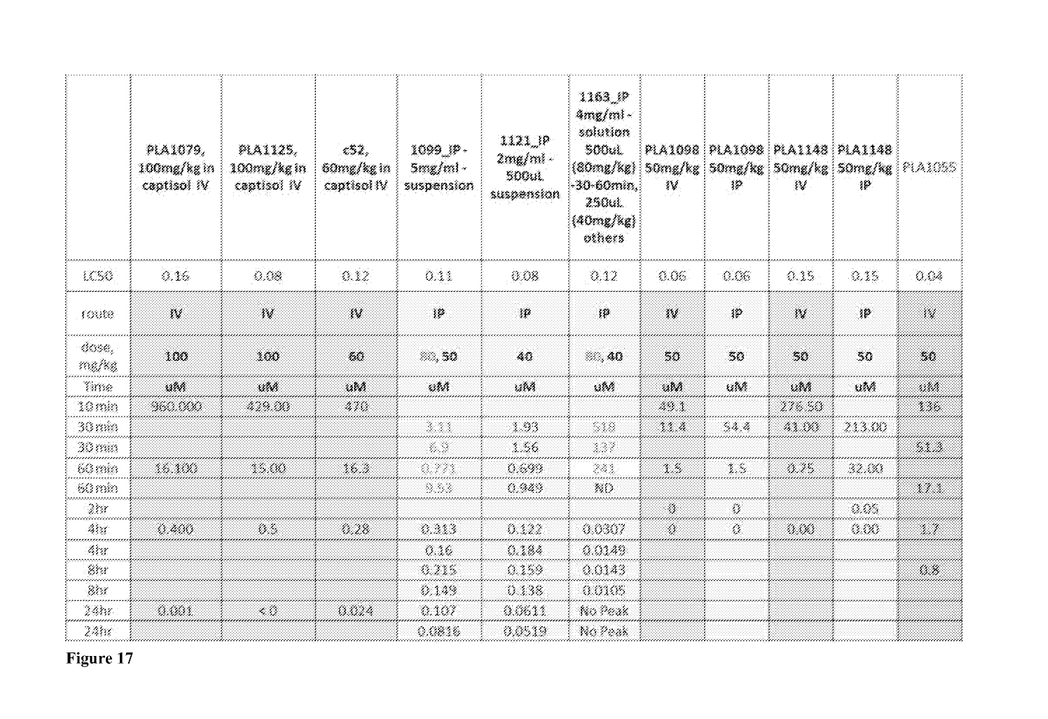

Summary of PK for tested compounds is shown in FIG. 17. Intra-tumor drug concentrations for all tested PLA compounds are shown in Table 2. LC50 data of in vitro 72 hours cytotoxicity assay are included in this table for comparison.

TABLE 2
Tissue drug concentrations for PLA compounds tested.

| | | Route | | | |
|---|---|---|---|---|---|
| Com- | | of | Tumor concentration | LC50, | Liver |
| pound | Dose | delivery | (nM) | nM | (nM) |
| c52 | 60 mg/kg | IP/IV | 21-24/0.5-4 nM | 150 | 23 |
| PLA1079 | 100 mg/kg | IP/IV | 24-48 nM | 120 | ND |
| PLA1125 | 100 mg/kg | IP/IV | 7-10 nM | 100 | ND |
| PLA1098 | 100 mg/kg | IP | 23-25 nM | 40 | 0 |
| PLA1148 | 50 mg/kg | IP | 0-90 nM | 200 | 50 |
| PLA1163 | 40 mg/kg | IP | 4-10 nM | 200 | 307 |

TABLE 3
PLA compounds selected for in vivo evaluation.

| | Reference No | State | Amount | Series | Storage |
|---|---|---|---|---|---|
| PLA01055-00- | PLA01055 | Powder | 29.7 | c52 | RT |
| 01 | | | | | |
| PLA01128-00- | PLA01128 | Powder | 39.5 | 219 | RT |
| 01 | | | | | |
| Pla01164-00-02 | Pla01164-02 | Powder | 49.7 | 219 | RT |
| PLA01171-00- | PLA01171 | Powder | 23.1 | 219 | RT |
| 01 | | | | | |
| PLA01173-00- | PLA01173-02 | Powder | 58.1 | 219 | RT |
| 02 | | | | | |

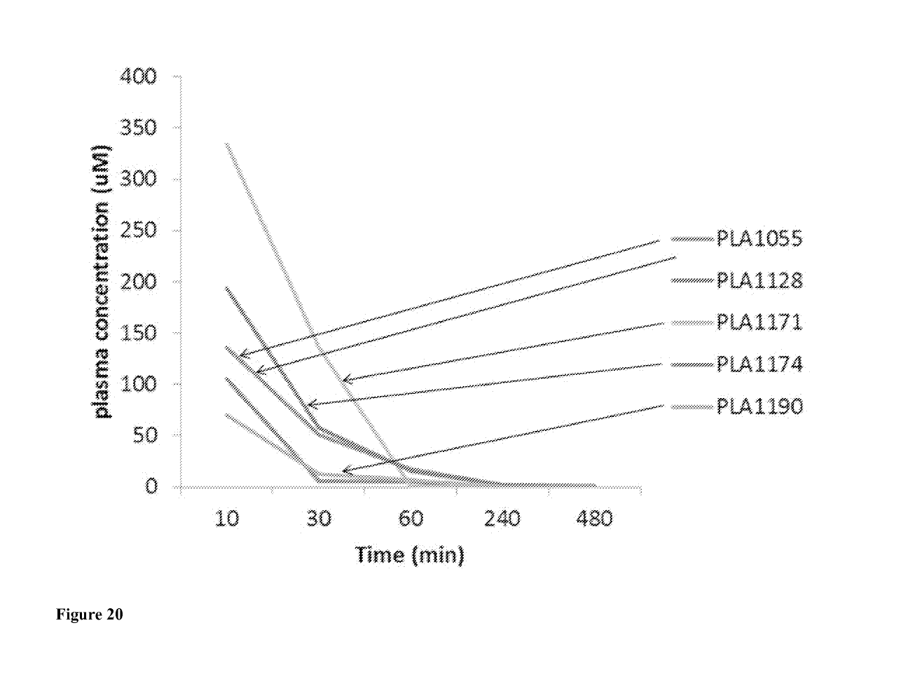

Formulations of the compounds PLA1055 and PLA1128, PLA1170, PLA1171 and PLA 1190 were developed. PK data for solubilized compounds are shown on FIG. 3. Compounds were tested for biological activity (Table 3) and 4 compounds were formulated for IV injections. Of the compounds tested for PK (FIG. 20), one showed low half-life. Another one, PLA1055, was detected up to 24 hours and at 8 hours at concentration slightly below 1 uM, the highest among the compounds tested. PLA1055 is also the most active compound in this group (LC50% 40 nM).

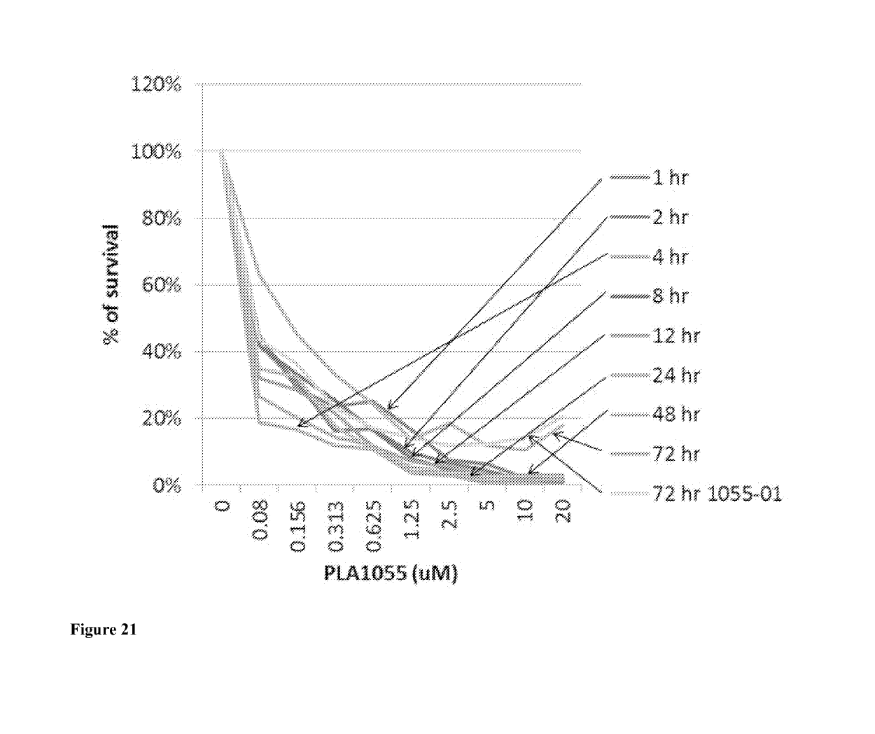

An in vitro experiment was run to detect the concentration of PLA1055 needed to kill all tumor cells in vitro (LC90) during different periods of time (30 minutes-72 hours, FIG. 21)). LC90 at 4-8 hours was 2.5 uM, what is similar to plasma concentrations at this point of time (FIG. 20).

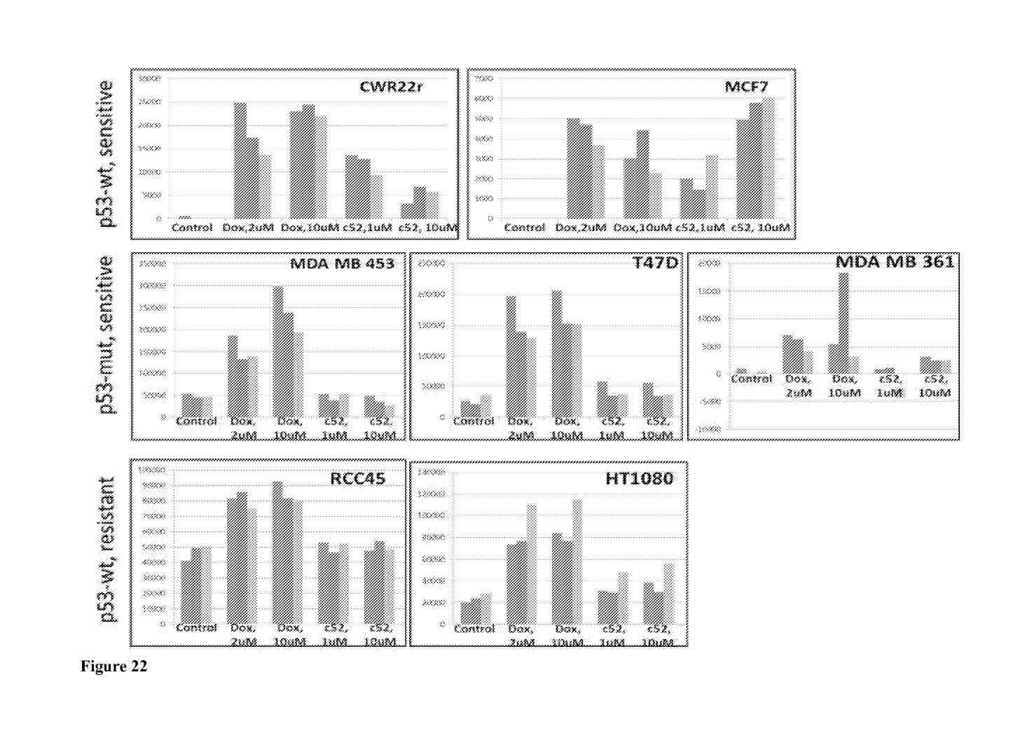

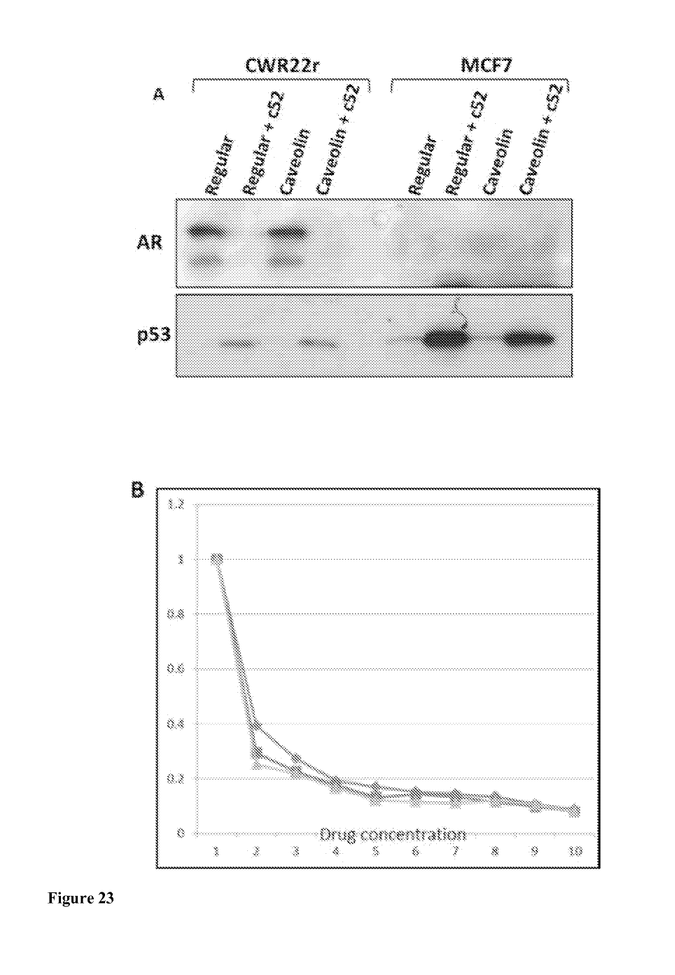

It was demonstrated that c52 induces apoptosis only in p53 wild type cells, although other sensitive cells die through non-apoptotic mechanism (FIG. 22). It was also found that replication stress happens in c52 treated cells only if they express AR. There are indications that AR may be important for replication in these cells. It was shown that Caveolin1-expressing cells were still were sensitive to c52, treatment with c52 still forced degradation of AR and activation of p53 (FIG. 23). Therefore, hypothesis of Caveolin1 responsibility for c52 sensitivity was eliminated.

PLA1098 demonstrated evidence of in vivo efficacy without any signs of toxicity.

It was established that c52 causes replication stress in AR positive sensitive cells. This stress leads to activation of p53 and death of tumor cells through apoptosis mechanism. Death of cells without AR or with mutant p53 undergoes through alternative mechanism.

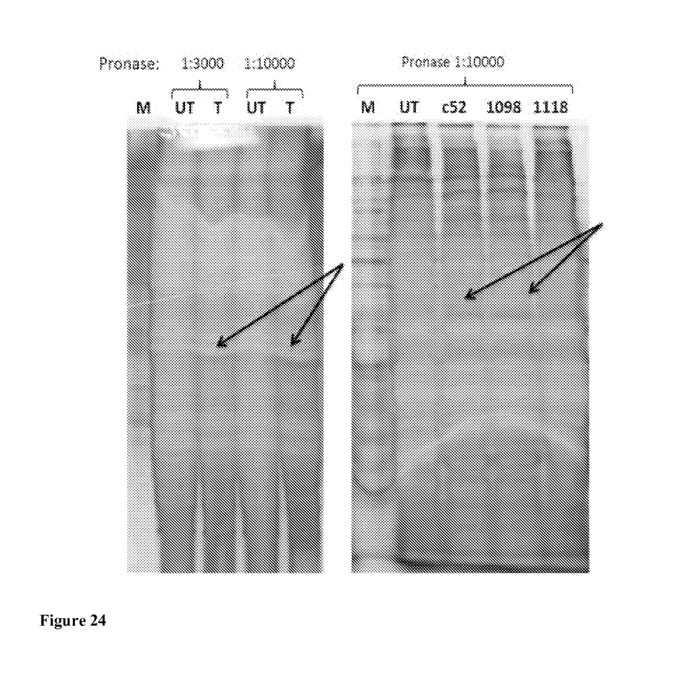

Target identification through affinity chromatography and sensitivity to proteolysis methods was undertaken. Experiments with different types of control were run, all of which demonstrated the presence of several bands resistant to proteolysis in lysates incubated either with c52 or with another active, but not with inactive compound. These bands were excised from gel and sent for protein sequencing. (FIG. 24).

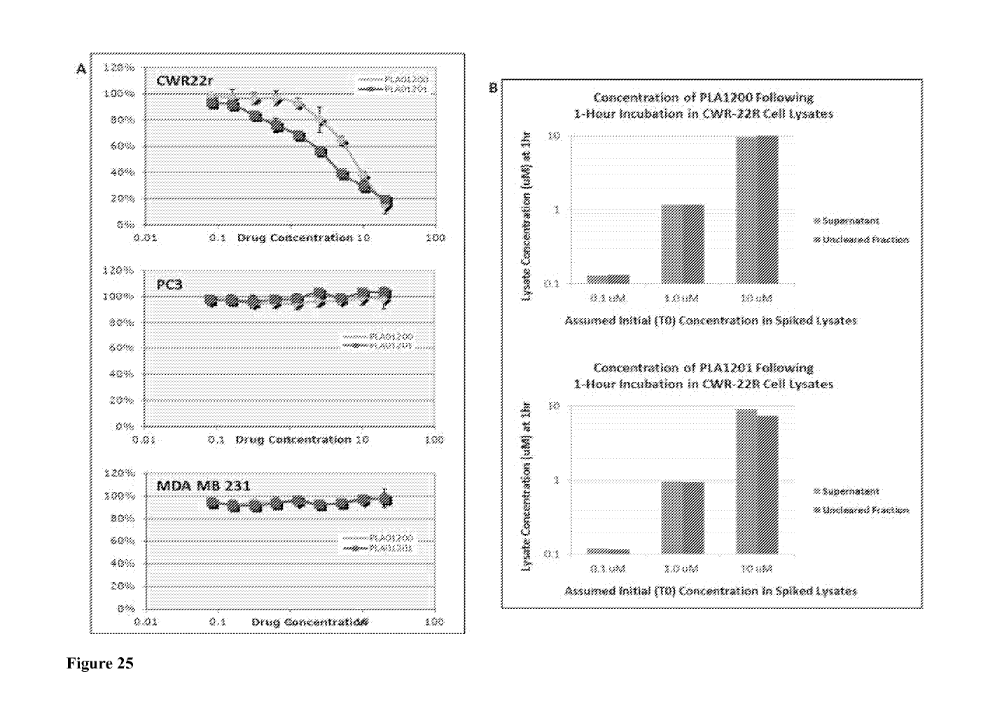

Two c52-like compounds with flexible linkers were synthesized. These compounds were tested for the presence of biological activity and stability in cell lysates. Both properties were confirmed. Biotin was attached to the flexible linker of one of the compounds to do affinity purification (FIG. 25).

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A compound having the following structure: 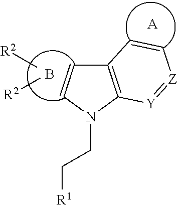
wherein R¹ is selected from the group consisting of a hydrogen atom, CH₃, CH₂F, CHF₂ and CF₃; R² is independently at each occurrence a hydrogen atom, halogen atom, —CN, —OH, substituted or unsubstituted alkyl, substituted or unsubstituted alkoxy group, —C(═O)N(R³)₂, —N(R³)₂, ketone, substituted or unsubstituted cycloalkyl group, or substituted or unsubstituted heterocycloalkyl group; Y and Z are independently a carbon or nitrogen atom; ring A is a substituted or unsubstituted 5 to 7 membered carbocyclic or heterocyclic ring; ring B is a substituted or unsubstituted 5 to 6 membered aryl or heteroaryl ring with 0 to 2 R² groups; and R³ is a hydrogen atom or substituted or unsubstituted alkyl group.

2. The compound of claim 1, wherein the compound has the following structure: 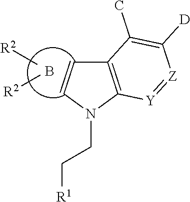
wherein C and D are replaced by the atoms of the following structures: 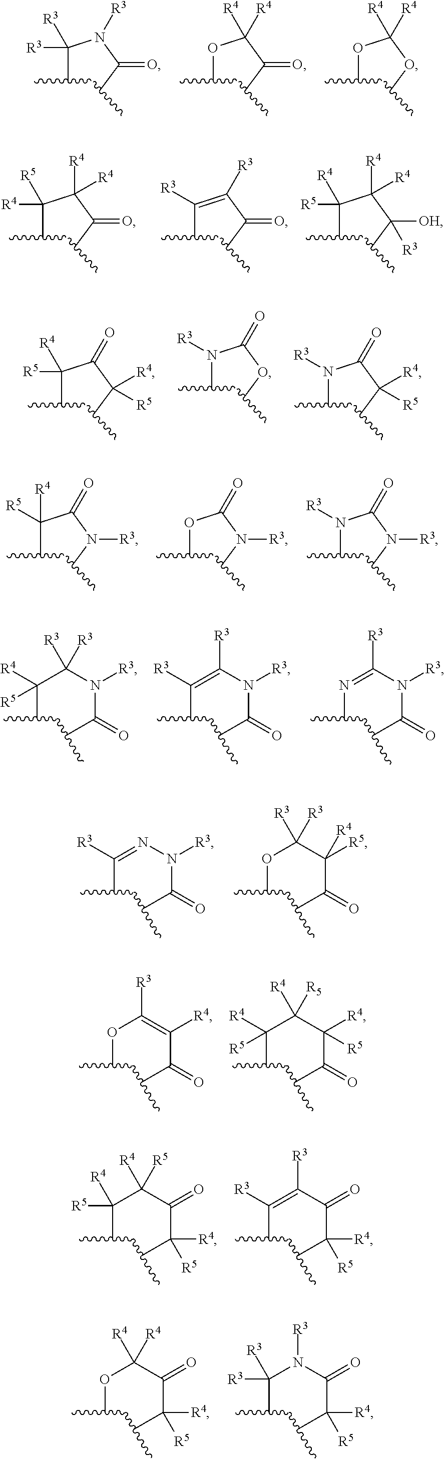 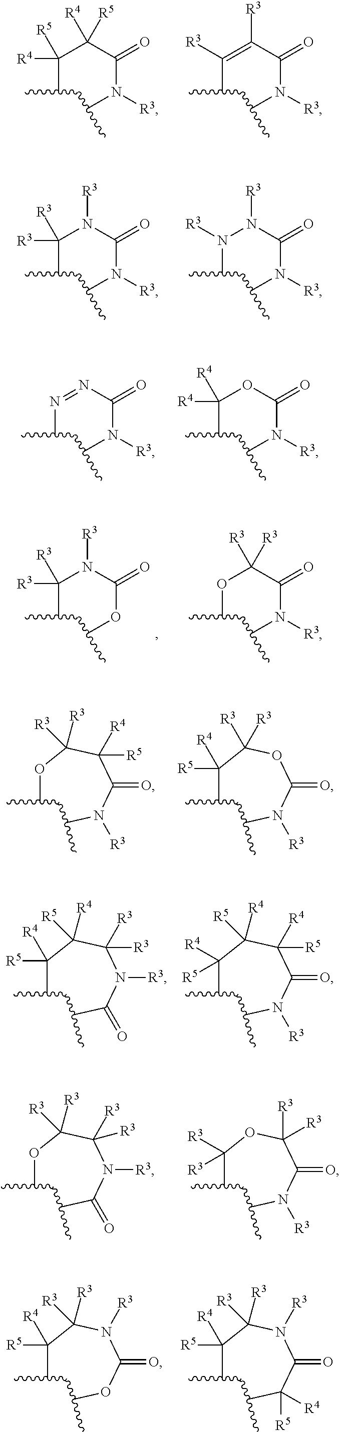
to
form a ring, where R³ is hydrogen atom or alkyl group, R⁴ is a hydrogen atom, halogen atom, or alkyl group, and R⁵ is a hydrogen atom, halogen atom, alkyl group, or an alkoxy group.

3. The compound of claim 1, wherein the compound has the following structure: 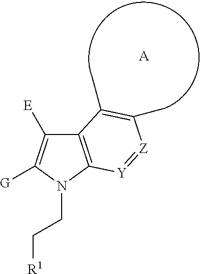
wherein E and G are replaced by the atoms of one the following structures 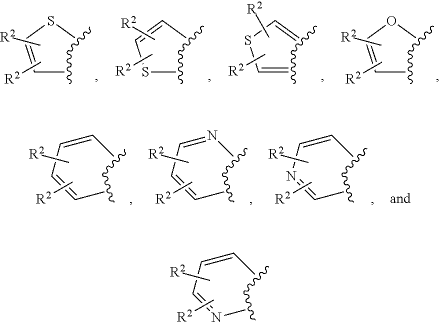
to form a ring.

4. The compound of claim 1, wherein the compound has the following structure:

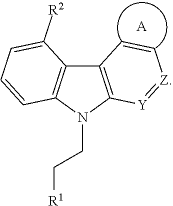

5. The compound of claim 1, wherein the compound has the following structure:

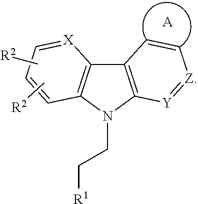

6. The compound of claim 1, wherein the compound has the following structure:

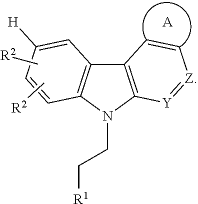

7. The compound of claim 1, wherein the compound has the following structure:

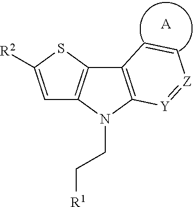

8. The compound of claim 1, wherein the compound has the following structure: 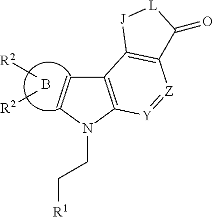
wherein J is an oxygen atom, —C(R⁴)₂, or —NR³, and L is —C(R⁴)₂ or —NR³.

9. The compound of claim 1, wherein the compound has the following structure: 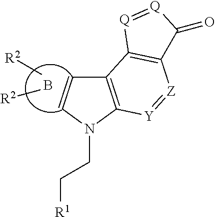
wherein each Q is independently —C(R³) or a nitrogen atom.

10. The compound of claim 1, wherein the compound has the following structure: 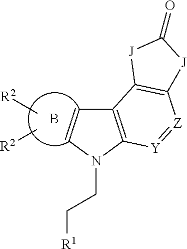
wherein each J is independently an oxygen atom, —C(R¹)₂, or —NR³ and at most one J is an oxygen atom.

11. The compound of claim 1, wherein the compound has the following structure: 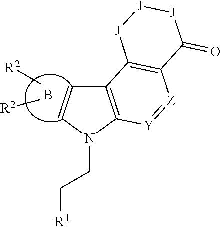
wherein each J is independently an oxygen atom, —C(R⁴)₂, or —NR³ and at most one J is an oxygen atom

12. The compound of claim 1, wherein the compound has the following structure: 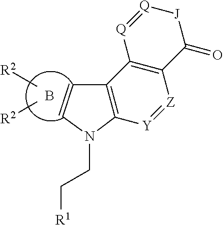
wherein each Q is independently —CR³ or a nitrogen atom, J is an oxygen atom, —C(R⁴)₂, or —NR³, and at least one Q is —CR³.

13. The compound of claim 1, wherein the compound has the following structure: 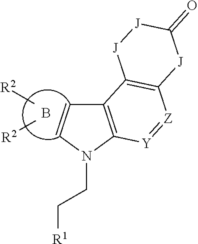
wherein each J is independently an oxygen atom, —C(R⁴)₂, or —NR³ and at most one J is an oxygen atom.

14. The compound of claim 1, wherein the compound has the following structure: 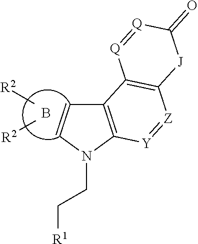
wherein each Q is independently —CR³ or a nitrogen atom, J is an oxygen atom, —C(R⁴)₂, or —NR³.

15. The compound of claim 1, wherein the compound has the following structure: 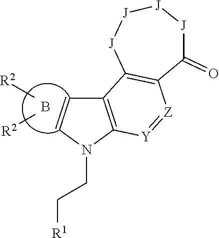
wherein each J is independently an oxygen atom, —C(R⁴)₂, or —NR³, at least one J is —C(R⁴)₂ and at most one J is an oxygen atom.

16. The compound of claim 1, wherein the compound has the following structure: 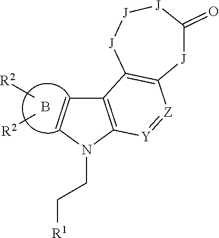
wherein each J is independently an oxygen atom, —C(R⁴)₂, or —NR³, at least one J is —C(R⁴)₂ and at most one J is an oxygen atom.

17. The compound of claim 1, wherein the compound has the following structure: 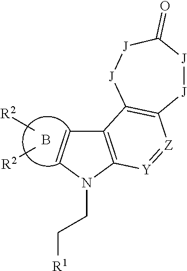
wherein each J is independently an oxygen atom, —C(R⁴)₂, or —NR³, and at most one J is an oxygen atom.

18. The compound of claim 1, wherein the compound has the following structure: 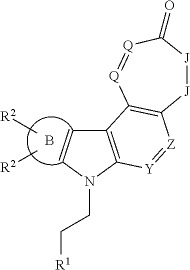
wherein each Q is independently —CR³ or a nitrogen atom, each J is independently an oxygen atom, —C(R⁴)₂, or —NR³, and at most one J is an oxygen atom.

19. The compound of claim 1, wherein the compound is selected from the following structures:

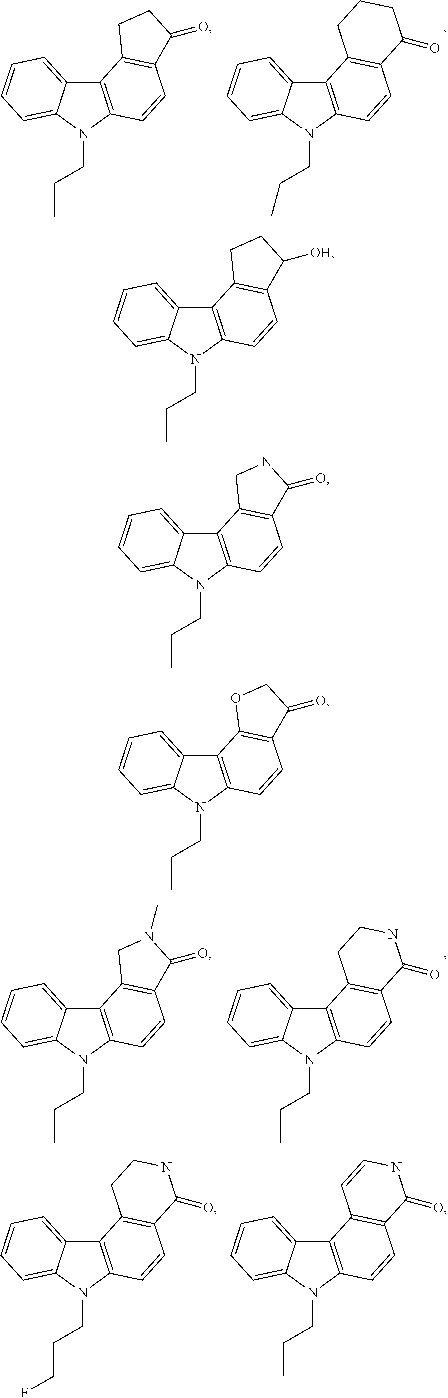

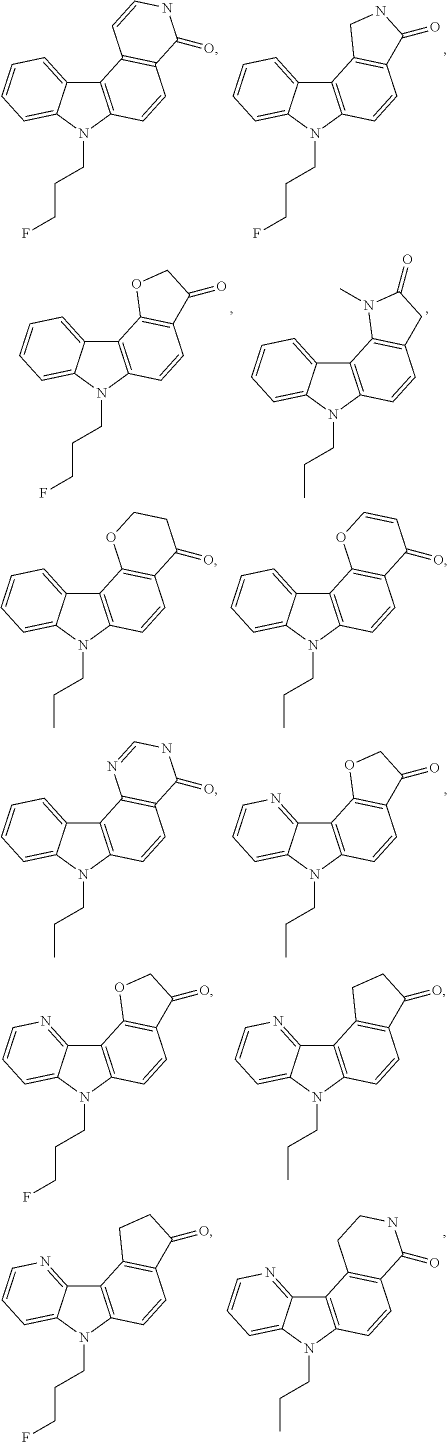

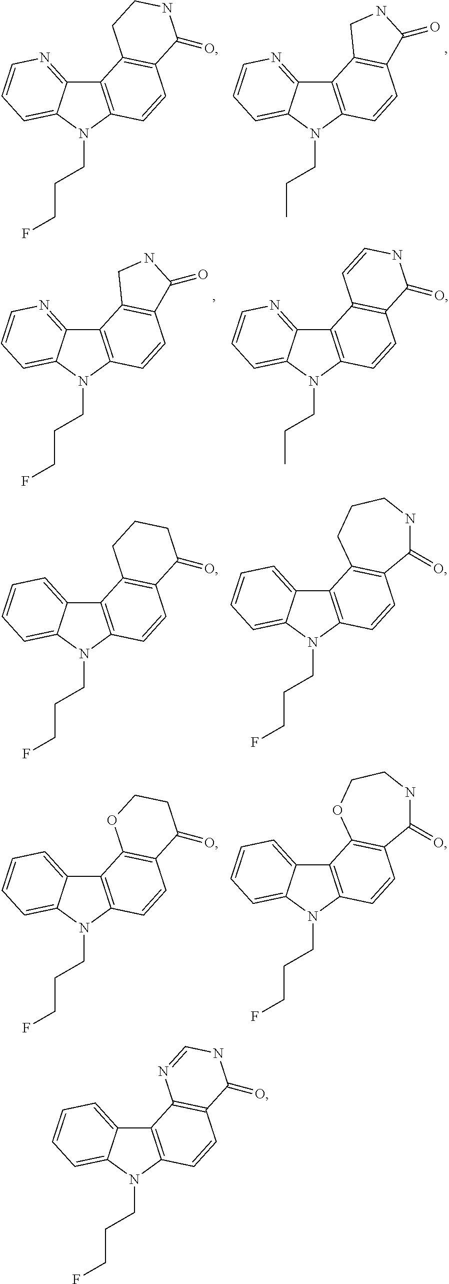

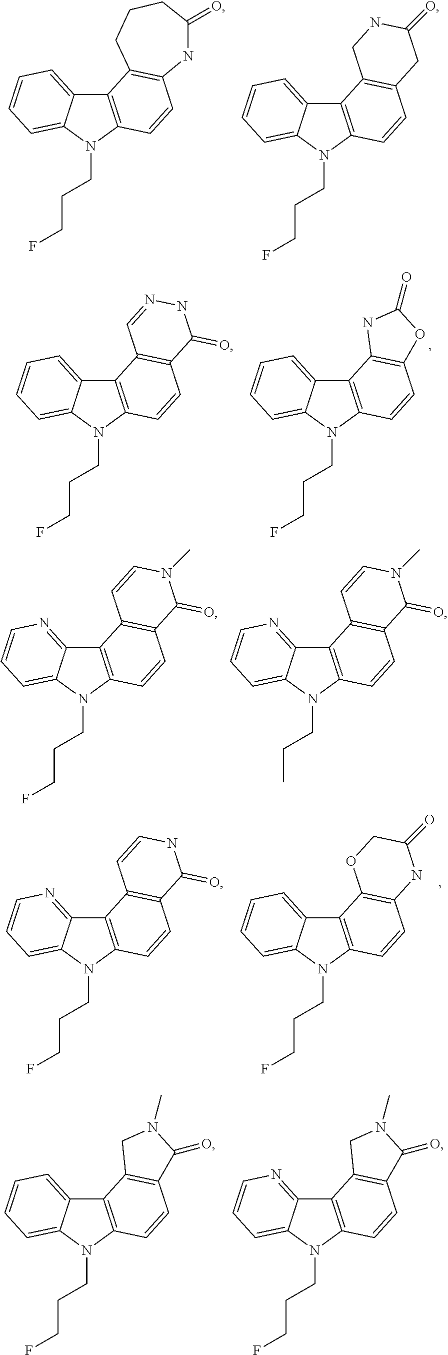

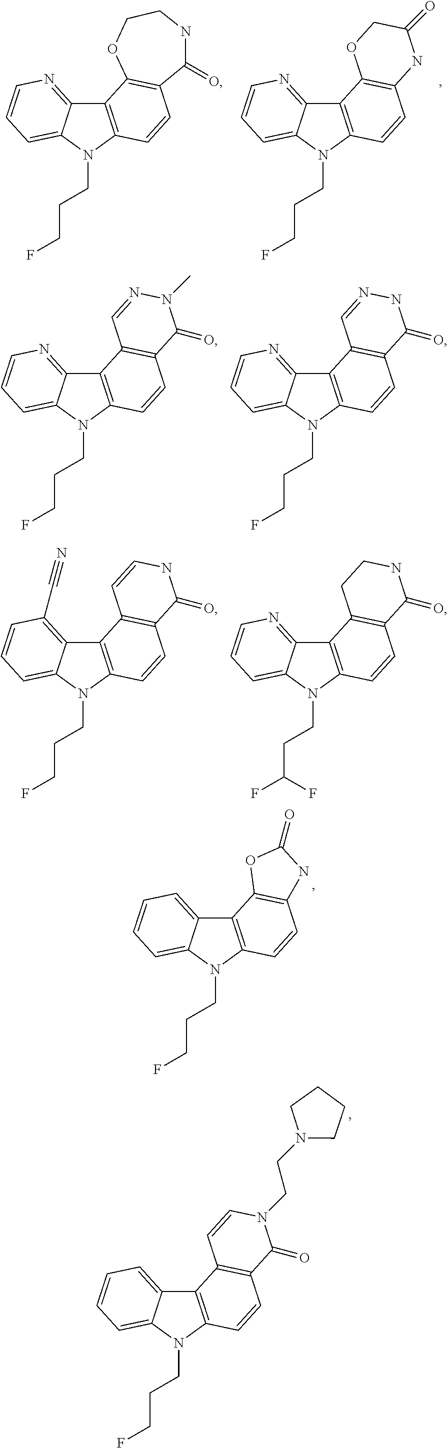

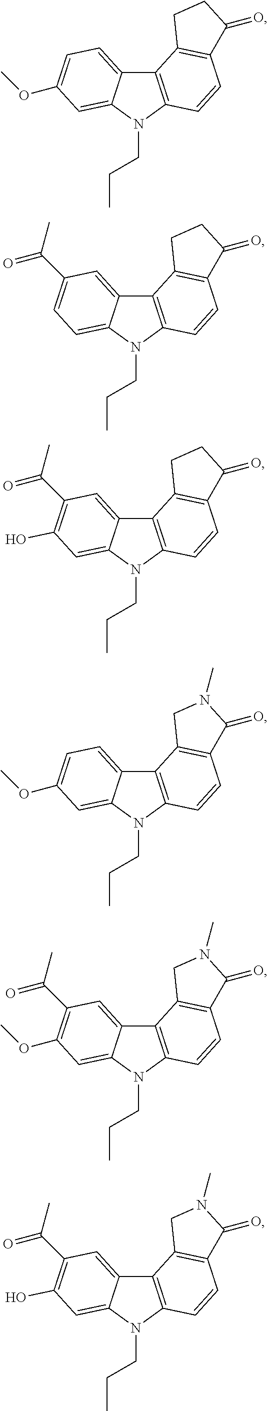 [SMILES: CCCn1c2cc(OC)ccc2c2c3c(ccc21)C(=O)CC3 ; CCCn1c2ccc(C(C)=O)cc2c2c3c(ccc21)C(=O)CC3 ; CCCn1c2cc(O)c(C(C)=O)cc2c2c3c(ccc21)C(=O)CC3 ; CCCn1c2cc(OC)ccc2c2c3c(ccc21)C(=O)N(C)C3 ; CCCn1c2cc(OC)c(C(C)=O)cc2c2c3c(ccc21)C(=O)N(C)C3 ; CCCn1c2cc(O)c(C(C)=O)cc2c2c3c(ccc21)C(=O)N(C)C3]

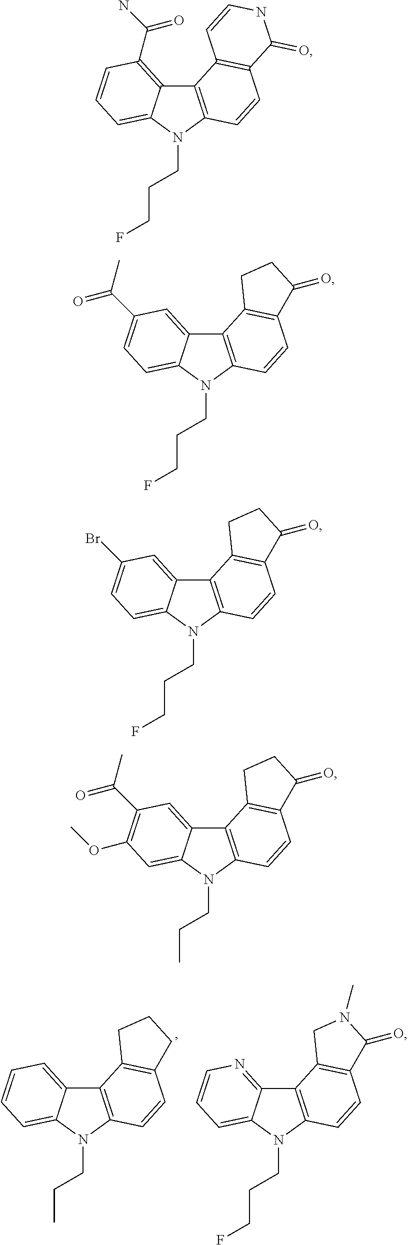

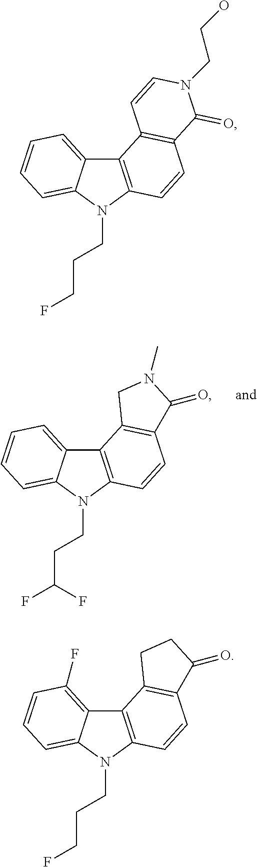 [SMILES: O=c1c2ccc3c(c2ccn1CCO)c1ccccc1n3CCCF ; CN1Cc2c(ccc3c2c2ccccc2n3CCC(F)F)C1=O ; "and" ; O=C1CCc2c1ccc1c2c2c(F)cccc2n1CCCF]

20. A method for inhibiting the growth of AR positive or negative cancer cells in an individual diagnosed with or suspected of having AR positive or negative cancer comprising administering to the individual a composition comprising a compound of claim 1.
